(19) United States
(12) Patent Application Publication
(10) Pub. No.: US 2004/0197347 A1
(43) Pub. Date: Oct. 7, 2004

(54) Methods for vaccine identification and compositions for vaccination comprising nucleic acid and/or polypeptide sequences of the herpesvirus family

(75) Inventors: Kathryn F. Sykes, Dallas, TX (US); Katherine S. Hale, Dallas, TX (US); Stephen A. Johnston, Dallas, TX (US)

Correspondence Address: FULBRIGHT & JAWORSKI L.L.P., 600 CONGRESS AVE., SUITE 2400, AUSTIN, TX, 78701, US

(73) Assignees: Board of Regents, The University of Texas System; MacroGenics, Inc.

(21) Appl. No.: 10/669,161

(22) Filed: Sep. 23, 2003

Related U.S. Application Data
(60) Provisional application No. 60/412,956, filed on Sep. 23, 2002.

(30) Foreign Application Priority Data
May 29, 2003 (KR) 2003-34306

(51) Int. Cl. A61K 39/12

(52) U.S. Cl. 424/186.1

(57) ABSTRACT

The instant invention relates to antigens and nucleic acids encoding such antigens obtainable by screening a herpesvirus genome, in particular an HSV-1 genome. In more specific aspects, the invention relates to methods of isolating such antigens and nucleic acids and to methods of using such isolated antigens for producing immune responses. The ability of an antigen to produce an immune response may be employed in vaccination or antibody preparation technique.

This application claims the benefit of U.S. Provisional Application Serial No. 60/412,956 Entitled “METHODS AND COMPOSITIONS FOR VACCINATION COMPRISING NUCLEIC ACID AND/OR POLYPEPTIDE SEQUENCES OF THE HERPESVIRUS FAMILY” filed Sep. 23, 2002.

The government owns rights in the present invention pursuant to DARPA Grant number MDA9729710013.

### BACKGROUND OF THE INVENTION

A. Field of the Invention

The present invention relates generally to the fields of vaccinology, immunology, virology, functional genomics, and molecular biology. More particularly, the invention relates to methods for screening and obtaining vaccines generated from the administration of gene expression libraries derived from a herpesvirus genome. In particular embodiments, it concerns methods and compositions for the vaccination of a subject against herpesvirus infections and diseases, wherein vaccination of the subject may be via compositions that contain single or multiple polypeptides or polynucleotides or variants thereof derived from part or all of the genes or similar sequences validated as protective or immunogenic by the described methods.

B. Description of Related Art

Purely on empirical grounds, Edward Jenner first demonstrated protective vaccination against an infectious disease in the 1790s. After observing that milkmaids did not contract smallpox, he intentionally infected a boy with cowpox then subsequently found him immune to smallpox infection. Since then, vaccines against measles, polio, anthrax, rabies, typhoid fever, cholera, and plague, and many other infectious agents have been developed. The methods of developing new vaccines vary and differ for each virus, bacterium, or other pathogen target; however, they have traditionally consisted of whole pathogens in an attenuated or killed form, as did Jenner's vaccine. Both social and economic considerations make vaccination the optimal method for protecting animals and humans against infectious diseases. However, vaccines are not available for many of the most serious human infectious diseases, including Malaria, tuberculosis, HIV, respiratory syncytial virus (RSV), Streptococcus pneumoniae, rotavirus, Shigella and other pathogens. There is a need to develop effective vaccines, yet for many pathogens vaccines are not readily produced. For example, the antigenic drift of influenza virus requires that new vaccines be constantly developed annually. Research efforts continue to try to identify effective vaccines for rabies (Xiang, et al, 1994), herpes (Rouse, 1995); tuberculosis (Lowrie, et al, 1994); HIV (Coney, et al, 1994) as well as many other diseases or pathogens.

Most currently available vaccines are composed of live/attenuated or killed pathogens (Ada, 1991). These whole-pathogen inocula elicit a broad immune response in the host. The strength of this approach is that no antigen identification is required, because all the components of the pathogen are presented to the immune system. However, this straightforward approach carries an inherent problem. Pathogenicity of the live/attenuated strain or its reversion to virulence is possible. At best, components of the pathogen that are not needed for the protective immune response are carried as baggage; alternatively some components may compromise protective immunity. In some instances, protective antigens maybe lost or denatured during the process of inactivation of the pathogen. Pointedly, pathogens become pathogenic by evolving or acquiring factors to defend themselves against or avoid a host immune system. In particular, many HSV genes are involved in immune evasion and pathogenesis, especially those that have been shown to be dispensable in vitro. In whole organism vaccines, whether live/attenuated or killed, the repertoire of antigens and their expression levels are controlled by the pathogen. Consequently, the host immune system is often not directed to the most protective antigen determinants. Another consideration is that when all the potential protective antigens of a pathogen are presented to the host, there are opportunities for the non-protective ones to cause deleterious side effects such as autoimmunity, toxicity, or interference with the response to the protective antigens.

Alternatives to the use of whole-pathogen vaccines include the use of a single immunodominant component or a small group of components for stimulation of a protective immune response in the host. Some component vaccines, such as tetanus toxoid, consist of an enriched, but not highly purified pathogen component. Others, consist of recombinant components, such as the hepatitis B vaccine. They have provided improved immunogenicity and safety, reduced side-reactivities, and easier quality control relative to whole organism vaccines. However, the antigens conferring the best protection are not always known, so the choice has often fallen to educated guessing or technical convenience, followed by further study. For example, subunits have been chosen as vaccine candidates on the basis that they correspond to components of the pathogen that i) generate high levels of antibodies, ii) are expressed on the pathogen surface or are secreted, iii) carry consensus major histocompatibitilty (MHC) binding sites, or iv) are abundant and easy to purify. Unfortunately these candidates must be unsystematically tested by trial and error, because broad-based functional screens for vaccine candidates are impractical using protein, peptide, or live vector delivery methods. This defines a more basic and unsolved problem of identifying the particular gene or genes of the pathogen that will express an immunogen capable of priming the immune system for rapid and protective response to pathogen challenge.

Certain non-viral pathogens and some viruses have very large genomes; for example, protozoa genomes contain up to about 10⁸ nucleotides, thus posing an expensive and time-consuming analytical challenge to identify or isolate effective immunogenic antigens. Evaluating the immune potential of the millions of possible determinants from even one pathogen, antigen by antigen, is a significant hurdle for new vaccine development.

In particular, new protective antigens need to be discovered against Herpesviridae, a family of viral pathogens. Herpesvirus (HSV) infections are increasingly common worldwide, with HSV types 1 and 2 (HSV-1, HSV-2) inflicting the greatest disease burden (Stanberry et al., 1997). Over the past 20 years the U.S. population has suffered a steep rise in HSV infections (Whitley and Miller, 2001; and Farrell et al., 1994) and the vast majority of the world population is infected with at least one member of the human Herpesvirus family (Kleymann et al., 2002). The viruses cause a variety of similar illnesses that are determined by the transmission route, infection site, dose, and host immune status (Whitley et al., 1998). A defining characteristic of HSVs is their acute phase infection, followed by life-long infection of neuronal cells. The Greek translation of their namesake is “creeping”, which describes their persistence and latency (Whitley and Roizman, 2001). Most adults harbor HSV-1 in their peripheral nervous systems in a latent state. Viral reactivation in the sensory ganglia is induced by stress and causes recurrent symptoms, lesions and viral shedding. HSV-1 is most often associated with orofacial infections, encephalitis and infections of the eye, which can cause blindness from resultant corneal scarring. HSV-2 is usually associated with genital infections, however primary genital herpes resulting from HSV-1 has become increasingly common (Whitley and Miller, 2001). Antiviral drugs including acylovir are the mainstays of current herpes therapy (Leung and Sacks, 2000). These treatments suppress episodic symptoms but are only effective with continuous administration, which is both demanding and encourages the emergence of resistant strains. Poignantly, the availability of these drugs has not prevented genital herpes from becoming the third most prevalent sexually transmitted disease in the world (Whitley, and Miller, 2001), and ocular herpes from becoming the second leading cause of blindness in industrialized countries. Rampant infection in the general population combined with severe disease in young and immune compromised hosts has stimulated efforts to develop a herpes vaccine (Bernstein and Stanberry, 1999).

While the ultimate goal of an HSV vaccine would be long-lasting protection from viral infection, the suppression of disease symptoms would also provide significant health benefits. One of the current goals for either a prophylactic or therapeutic vaccine is to reduce clinical episodes and viral shedding from primary and latent infections. Three categories of prophylactic vaccines have been tested in clinical trials with disappointing results i) whole virus, ii) protein subunit and iii) gene-based subunit vaccines (Stanberry et al., 2000). In the 1970s a number of killed virus vaccines were explored, none of which were efficacious. More recently an attenuated HSV was found to be poorly immunogenic. A replication incompetent virus is being used in clinical trials, but the clinical use of a replication incompetent virus raises safety concerns. Subunit vaccines based on two recombinant glycoproteins have been clinically evaluated in combination with different adjuvant formulations. One developed by Chiron contains truncated forms of both gD₂ and gB₂ of HSV-2, purified from transfected CHO cells and formulated in the adjuvant MF59. Another developed by Glaxo-Smithkline (GSK) contains a truncated gD₂ formulated with adjuvants alum and 3-O-deacylated monophosphoryl lipid A (MPL). Both vaccines were immunogenic and well tolerated in phase I/II trials. However in phase III analyses, the Chiron vaccine showed no overall efficacy against HSV-2 seroconversion and work was discontinued. The GSK vaccine showed significant efficacy (73-74%) in HSV-1, HSV-2 seranegative women volunteers but no efficacy in men. Also, a genetic vaccine using gD₂ was placed in a phase I trial, and the immunogenicity data are currently being analyzed.

While even limited vaccine efficacy would beneficially impact HSV sufferers, these trials are testing only a small number of vaccine possibilities. This is because the vaccine discovery has not been systematic. Pursuance of a whole-virus vaccine assumes that presentation of the pathogen itself to the immune system will generate optimal immunity. Indeed the breadth and duration of immune responses to whole pathogen vaccines historically have been better than subunit vaccines. However, pathogenicity of the vaccine strain must be considered. Subunit vaccines, to date, have been selected for vaccine testing based on their assumed importance in disease pathogenesis and immunogenicity during infection. These approaches have identified one candidate against HSV with limited efficacy in some but no efficacy in other formulations. Thus, new and improved methodologies for herpesvirus vaccine discovery are needed to protect against herpes diseases.

### SUMMARY OF THE INVENTION

In certain embodiments of the invention two methods were employed to systematically screen the coding sequences of HSV-1 for protective antigens. Random ELI (RELI), as previously demonstrated, provided novel candidates. However, the development of microbial genomics, high-throughput oligonucleotide synthesis, and the invention of linear expression elements (LEEs) enable the screening power of ELI to be increased in terms of breadth and speed. Various embodiments of the invention use a novel directed ELI (DELI) method and idnetify various novel candidates from the HSV-1 genome. Using the sequence of a pathogen's genome, primers can be designed to amplify genes by polymerase chain reaction (PCR) or other nucleic acid amplification techniques. Inexpensive oligonucleotide synthesis in microtiter-formats makes production of primer-sets for entire genomes of pathogen practical. The construction of each PCR-amplified ORF into an expression vector for genetic immunization is required to perform ELI. To avoid several hundred anticipated cloning steps and the associated artifacts, the inventors developed linear expression elements. (U.S. Pat. No. 6,410,241, incorporated herein by reference). In the LEE protocol, PCR-amplified ORFs are covalently or non-covalently linked to advantageous promoter and terminator elements then directly delivered into animals for expression by genetic immunization. This alternative to cloning dramatically streamlines the process of obtaining expression vectors. Genes of many different lengths from many sources have been PCR-amplified and efficiently linked to different expression elements using a variety of methods. Quantitation of LEE and plasmid-borne gene expression in vivo has shown that activity levels are nearly identical. Immune responses and protection-assay readouts that are generated by genetic antigens delivered as LEEs and plasmids are indistinguishable.

These technologies have been combined to design new ELI screening methods that significantly increase the sensitivity while decreasing the time, expense, and variability of the process. Because each library member is sequence-defined, the components of each sub-library pool can be designed, complete genomic coverage is ensured, and constructs are positioned for proper expression. This circumvents a statistically invoked requirement for library clone redundancy and for carrying unexpressed DNA baggage. Construction of sequence-directed fragments (directed amplification) decreases library sizes, mouse numbers, sibbing rounds, and mistakes. Each defined gene of a pathogen can now be generated to create an ordered array representing the full coding capacity of the pathogen in microtiter plates. The gene arrays are expressed without E. coli-based plasmid propagation, thereby saving time and resources, and avoiding cloning-associated pitfalls.

The present invention overcomes various difficulties and problems associated with immunization against viruses of the Herpesvirus family. Various embodiments of the invention include compositions comprising herpesvirus polypeptides and polynucleotides, which encode such polypeptides, that may be used as antigens for immunization of a subject. The present invention may also include vaccines comprising antigens derived from other viruses of the Herpesvirus family, as well as methods of vaccination using such vaccines. Vaccine compositions and methods may be broadly applicable for immunization against a variety of herpesvirus infections and the diseases and disorders associated with such infections. An antigen, as used herein, is a substance that induces an immune response in a subject. In particular, compositions and methods may include polypeptides and/or nucleic acids that encode polypeptides obtained by functionally screening the genome of a virus or viruses of the Herpesvirus family, e.g., HSV-1, HSV-2, varicella zoster virus (VZV), bovine herpes virus (BHV), equine herpes virus (EHV), cytomegalovirus (CMV), Cercopithecine herpes virus (CHV or monkey B virus), or Epstein-Barr virus (EBV).

Certain embodiments of the invention include isolated polynucleotides derived from members of the Herpesvirus family. In some embodiments, polynucleotides may be isolated from viruses of the Alphaherpesvirus sub-family, in particular HSV-1, HSV-2, or other members of the simplexvirus genus. Polynucleotides may include but are not limited to nucleotide sequences comprising the sequences as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115; or a complement, a fragment, or a closely related sequence thereof. In additional embodiments, the invention may relate to such polynucleotides comprising a region having a sequence comprising at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 125, 150, 200, or more contiguous nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69 SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115; a complement, or fragment thereof, as well as any intervening lengths or ranges of nucleotides. In some specific embodiments, the invention relates to, but is not limited, to polynucleotides comprising full length, fragments of, variants of, or closely related sequences of specific nucleic acids encoding UL1 (SEQ ID NO:7); UL17 (SEQ ID NO:39); UL28 (SEQ ID NO:63); or US3 (SEQ ID NO:105). Even more specific embodiments are related to the specific fragments, further fragments, variants, or closely related sequences of the nucleic acids of: UL1 set forth in SEQ ID NO:5; UL17 set forth in SEQ ID NO:37; UL28 set forth in SEQ ID NO:59; and US3 set forth in SEQ ID NO:103.

A herpesvirus polynucleotide may be isolated from genomic DNA or a genomic DNA expression library but it need not be. For example, the polynucleotide may also be a sequence from one species that is determined to be protective based on the protective ability of a homologous sequence in another species. For example, the polynucleotide could be a sequence selected from a Varicellovirus genus of the same Alphaherpesvirus sub-family (Alphaherpesvirnae) or a different sub-family such as the Betaherpesvirus (Betaherpesvirnae) sub-family, or Gammaherpesvirus (Gammaherpesvirinae) sub-family that was determined to be protective after analysis of the respective genomic sequence(s) for homologs of HSV-1 that had previously been shown to be protective in an animal or human subject. As discussed below, the polynucleotides need not be of natural origin, or to encode an antigen that is precisely a naturally occurring herpesvirus antigen.

In many embodiments, a polynucleotide encoding a herpesvirus polypeptide may be comprised in a nucleic acid vector, which may be used in certain embodiments for immunizing a subject against a herpesvirus (e.g., genetic immunization). In various embodiments a genetic immunization vector may express at least one polypeptide encoded by a herpesvirus polynucleotide. In other embodiments, the genetic immunization vector may express a fusion protein comprising a herpesvirus polypeptide. A polypeptide expressed by a genetic immunization vector may include a fusion protein comprising a herpesvirus polypeptide, wherein the fusion protein may comprise a heterologous antigenic peptide, a signal sequence, an immunostimulatory peptide, an oligomerization peptide, an enzyme, a marker protein, a toxin, or the like. A genetic immunization vector may also, but need not, comprise a polynucleotide encoding a herpesvirus/mouse ubiquitin fusion protein.

A genetic immunization vector, in certain embodiments, will comprise a promoter operable in eukaryotic cells, for example, but not limited to a CMV promoter. Such promoters are well known to those of skill in the art. In some embodiments, the polynucleotide is comprised in a viral or plasmid expression vectors. A variety of expression systems are well known. Expression systems include, but are not limited to linear or circular expression elements (LEE or CEE), expression plasmids, adenovirus, adeno-associated virus, retrovirus and herpes-simplex virus, PVAX1™ (Invitrogen); pCI neo, pCI, and pSI (Promega); Adeno-X™ Expression System and Retro-X™ System (Clontech) and other commercially available expression systems. The genetic immunization vectors may be administered as naked DNA or incorporated into viral, non-viral, cell-mediated, pathogen mediated or by other known nucleic acid delivery vehicles or vaccination methodologies.

In other embodiments, a polynucleotide may encode one or more antigens that may or may not be the same sequence. A plurality of antigens may be encoded in a single molecule in any order and/or a plurality of antigens may be encoded on separate polynucleotides. A plurality of antigens may be administered together in a single formulation, at different times in separate formulations, or together in separate formulations. An expression vector for genetic immunization may comprise at least 1, 2, 3, 4, 5, 6, 7, 8, 9, 10 or more polynucleotides or fragments thereof encoding at least 1, 2, 3, 4, 5, 6, 7, 8, 9, 10 or more antigens derived from one or more virus of the Herpesvirus family, and may include other antigens or immunomodulators from 1, 2, 3, 4, 5, 6, 7, 8, 9, 10, or more other pathogens as well.

Various embodiments of the invention may include viral polypeptides, including variants or mimetics thereof, and compositions comprising viral polypeptides, variants or mimetics thereof. Viral polypeptides, in particular herpesvirus polypeptides, include, but are not limited to amino acid sequences set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116; fragments, variants, or mimetics thereof, or closely related sequences. In additional embodiments, the invention may relate to polypeptides comprising a region having an amino acid sequence comprising at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 125, 150, 200, or more contiguous amino acids, as well as any intervening lengths or ranges of amino acids, in common with at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or SEQ ID NO:72; a complement, or fragment thereof. In some specific embodiments, the invention relates to, but is not limited, polypeptides comprising full length, fragments of, variants of, mimetics of, or closely related sequences of the amino acid sequences of UL1 (SEQ ID NO:8); UL17 (SEQ ID NO:40); UL28 (SEQ ID NO:64); or US3 (SEQ ID NO:106). Even more specific embodiments are related to the specific fragments, further fragments, variants, mimetics, or closely related sequences of: UL1 set forth in SEQ ID NO:6; UL17 set forth in SEQ ID NO:38; UL28 set forth in SEQ ID NO:60; and US3 set forth in SEQ ID NO:104.

Additional embodiments of the invention also relate to methods of producing such polypeptides using known methods, such as recombinant methods.

Polypeptides of the invention may be synthetic, recombinant or purified polypeptides. Polypeptides of the invention may have a plurality of antigens represented in a single molecule. The antigens need not be the same antigen and need not be in any particular order. It is anticipated that polynucleotides, polypeptides and antigens within the scope of this invention may be synthetic and/or engineered to mimic, or improve upon, naturally occurring polynucleotides or polypeptides and still be useful in the invention. Those of ordinary skill will be able, in view of the specifications, to obtain any number of such compounds.

Various embodiments of the invention include vaccine compositions. A vaccine composition may comprise (a) a pharmaceutically acceptable carrier; and (b) at least one viral antigen or nucleic acid encoding a viral antigen. In certain embodiments of the invention the vaccine may be against viruses of the Herpesvirus family. In other embodiments, a vaccine may be directed towards a member of the Alphaherpesvirus sub-family and in particular HSV-1, HSV-2, or VZV. In some embodiments, an HSV antigen has a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116; fragments, variants, or mimetics thereof, or closely related sequences. In other specific embodiments, the vaccine compostion comprises a nucleic acid encoding such an HSV antigen, including but not limited to nucleotide sequences comprising the sequences as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115; or a complement, a fragment, or a closely related sequence thereof. In some specific embodiments, the invention relates to, but is not limited, to vaccine compositions comprising full length, fragments of, variants of, mimetics of, or closely related sequences of the nucleic acid and amino acid sequences of UL1 (SEQ ID NO:7 and SEQ ID NO:8); UL17 (SEQ ID NO:39 and SEQ ID NO:40); UL28 (SEQ ID NO:63 and SEQ ID NO:64); or US3 (SEQ ID NO:105 and SEQ ID NO:106). Even more specific embodiments are related to the specific fragments, further fragments, variants, mimetics, or closely related sequences of: UL1 set forth in SEQ ID NO:5 and SEQ ID NO:6; UL17 set forth in SEQ ID NO:37 and SEQ ID NO:38; UL28 set forth in SEQ ID NO:59 and SEQ ID NO:60; and US3 set forth in SEQ ID NO:103 and SEQ ID NO:104.

In certain embodiments of the invention a vaccine may comprise: (a) a pharmaceutically acceptable carrier, and (b) at least one polypeptide and/or polynucleotide encoding a polypeptide having a herpesvirus sequence, including a fragment, variant or mimetic thereof. Herpesvirus polypeptides and/or polynucleotides include, but are not limited to HSV polypeptides or polynucleotides; fragments thereof, or closely related sequences. In some embodiments a herpesvirus polypeptide or polynucleotide may be an HSV-1 sequence.

The vaccines of the invention may comprise multiple polynucleotide sequences and/or multiple polypeptide sequences. In some embodiments, the vaccine will comprise at least a first polynucleotide encoding a polypeptide or a polypeptide having a herpesvirus sequence. Other embodiments, include at least a second, third, fourth, and so on, polynucleotide or polypeptide, wherein a first polynucleotide or polypeptide and a second or subsequent polynucleotide or polypeptide have different sequences. In more specific embodiments, the first polynucleotide may have a sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115; a complement, or fragment thereof and/or encode a polypeptide sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116; fragments, variants, or mimetics thereof, or closely related sequences. In other embodiments antigenic fragments may be presented in a multi-epitope format, wherein two or more antigenic fragments are engineered into a single molecule.

In various embodiments, the invention relates to methods of isolating herpesvirus (e.g., HSV-1, HSV-2, VZV, BHV, EHV, CMV, or CHV) antigens and nucleic acids encoding such, as well as methods of using such isolated antigens for producing an immune response in a subject. Antigens of the invention may be used in vaccination of a subject against a herpesvirus infection or herpes disease.

Embodiments of the invention may include methods of immunizing an animal comprising providing to the animal at least one herpesvirus antigen or antigenic fragment thereof, in an amount effective to induce an immune response. A herpesvirus antigen can be derived from HSV-1, HSV-2, or any other Herpesvirus species. As discussed above, and described in detail below, the herpesvirus antigens useful in the invention need not be native antigens. Rather, these antigens may have sequences that have been modified in any number of ways known to those of skill in the art, so long as they result in or aid in an antigenic or immune response.

In various embodiments of the invention, an animal or a subject is a mammal. In some cases a mammal may be a mouse, horse, cow, pig, dog, or human. Alternatively, a subject may be selected from chickens, turtles, lizards, fish and other animals susceptible to herpesvirus infection. In preferred embodiments, an animal or subject is a human.

Alternatively, these methods may be practiced in order to induce an immune response against a Herpesvirus species other than the simplexvirus genus, HSV, for example, but not limited to, cytomegalovirus (CMV), and/or Varicella Zoster Virus/human herpesvirus 3 (VZV).

In other aspects of the invention, methods of screening at least one test polypeptide or test polynucleotide encoding a polypeptide for an ability to produce an immune response comprising (i) obtaining at least one test polypeptide or test polynucleotide by (a) modifying the amino acid sequence of a known antigenic polypeptide or polynucleotide sequence of a polynucleotide encoding a known antigenic polypeptide; (b) obtaining a homolog of a known antigenic sequence of a polynucleotide encoding such a homolog, or (c) obtaining a homolog of a known antigenic sequence or a polynucleotide encoding such a homolog and modifying the amino acid sequence of the homolog or the polynucleotide sequence of the polynucleotide encoding such a homolog; and (ii) testing the test polypeptide or test polynucleotide under appropriate conditions to determine whether the test polypeptide is antigenic or the test polynucleotide encodes an antigenic polypeptide are contemplated. The test polypeptide may comprise a modified amino acid sequence or a homolog of a least one polypeptide as described herein or a fragment thereof. The test polypeptide may comprise an amino acid sequence of at least one of amino acid sequences described above or a fragment thereof, which sequence has been modified.

In certain embodiments, the method may comprise obtaining a test polynucleotide. The test polynucleotide may comprise a polynucleotide encoding a modified amino acid sequence of or a homolog of at least one polypeptide having a sequence as described herein or a fragment thereof. Embodiments may include obtaining the test polynucleotide comprising modifying the polynucleotide sequence of at least one of the nucleic acid sequences described herein or a fragment thereof.

In various embodiments, methods may further comprise identifying at least one test polypeptide as being antigenic or at least one test polynucleotide as encoding an antigenic polypeptide. The identified antigenic polypeptide or the polynucleotide encoding an antigenic polypeptide may be comprised in a pharmaceutical composition. The identified antigenic polypeptide or polynucleotide encoding an antigenic polypeptide may be used to vaccinate a subject. In particular embodiments, the subject is vaccinated against a herpesvirus. In a preferred embodiment, the herpesvirus is HSV-1. In other embodiments the subject is vaccinated against a non-herpesvirus disease.

In yet another aspect of the invention, methods of preparing a vaccine comprising obtaining an antigenic polypeptide or a polynucleotide encoding an antigenic polypeptide as determined to be antigenic by any of methods described herein, and placing the polypeptide or polynucleotide in a vaccine composition is contemplated.

Also contemplated are methods of vaccinating a subject comprising preparing a vaccine of composition of the invention and vaccinating a subject with the vaccine. In certain embodiments methods of treating a subject infected with a pathogen comprising administering a vaccine composition comprising at least one herpesvirus antigen or fragment thereof, or at least one polynucleotide encoding a herpesvirus antigen or a fragment thereof is contemplated. The vaccine composition may include, but is not limited to a genetic vaccine, a polypeptide vaccine, a cell-mediated vaccine, an attenuated pathogen vaccine, a live-vector vaccine, an edible vaccine, a killed pathogen vaccine, a purified sub-unit vaccine, a conjugate vaccine, a virus-like particle vaccine, or a humanized antibody vaccine. In particular embodiments, the vaccine composition comprises a polynucleotide encoding at least one herpesvirus antigen or fragment thereof as described herein. In various embodiments, the vaccine composition comprises at least one herpesvirus antigen or fragment thereof as described above.

Certain embodiments include methods of raising a therapeutic immune response against reactivation disease comprising administering a vaccine composition comprising at least one herpesvirus antigen or fragment thereof, as described above, or at least one polynucleotide encoding a herpesvirus antigen or a fragment thereof, also as described above.

In still a further aspect of the invention includes methods of passive immunization comprising administering at least one antigen binding agent reactive to one or more herpesvirus antigen to a subject. The herpesvirus antigen may comprise an amino acid sequence of at least one polypeptide, peptide or variant thereof as described herein. An antigen binding agent may include, but is not limited to an antibody, an anticalin or an aptamer.

In certain embodiments, methods for vaccination include administering a priming dose of a herpesvirus vaccine composition. The priming dose may be followed by a boost dose. In various embodiments, the vaccine composition is administered at least once, twice, three times or more. Vaccination methods may include (a) administering at least one nucleic acid and/or polypeptide or peptide vaccine composition and then (b) administering at least one polypeptide and/or nucleic acid vaccine composition.

Certain aspects of the invention may include methods of detecting Herpesvirus and/or antibodies to a herpesvirus comprising: (a) admixing an antibody that is reactive against an antigen having an amino acid sequence as set forth above with a sample; and (b) assaying the sample for antigen-antibody binding.

In further aspects, regardless of the source of nucleic acid encoding an antigen, the method of directed ELI (DELI) may be used. Exemplary methods of screening at least one, two, three, four, five, six, seven, ten, twenty, fifty, one hundred five hundred, thousands and hundreds of thousands of open reading frames, including all intergers therebetween, to determine whether it encodes a polypeptide with an ability to generate an immune response in an animal may comprise preparing in vitro at least one linear or circular expression element comprising an open reading frame linked to a promoter by amplification or synthesis of a known or predicted open reading frame; introducing the at least one linear or circular expression element into a cell within an animal with or without intervening cloning or bacterial propagation; and assaying to determine whether an immune response is generated in the animal by expression of a polypeptide encoded by the open reading frame in the expression element. In certain embodiments, the open reading frame can be produced in vivo and then non-covalently linked to the promoter in vitro. In various embodiments, the linear or circular expression element may further comprise a terminator linked to the open reading frame. The open reading frame may be derived from a pathogen RNA, DNA, and/or genomic nucleotide sequence. The pathogen can be a virus, bacterium, fungus, alga, protozoan, arthropod, nematode, platyhelminthe, or plant. In certain embodiments, the preparing of the expression element may comprise non-covalently or covalently linking the promoter and/or terminator to the open reading frame. The preparation of the expression element may comprise using polymerase chain reaction, or other nucleic acid amplification technique, and/or nucleic acid synthesis methods known in the art. In various embodiments, preparing the expression element can comprise chemical synthesis of the open reading frame. The method can further comprise identifying and/or isolating an antibody produced by the animal and directed against the polypeptide encoded by the open reading frame. In certain emdoiments, the linear or circular expression element may be injected into the animal. In various embodiments, the animal is protected from the challenge with the pathogen. The method can comprise identifying one or more antigens conferring protection to the animal.

In certain embodiments of the invention, the methods comprise generating chimeric DNAs for LEE/CEE production and include, but are not limited to generating complementary, single-stranded overhangs for non-covalent linkage, which can be subsequently turned into covalent attachments, if desired. Non-limiting examples of methods for linking or attachment of nucleic acid elements include dU/UDG, rU/Rnase, T4 polymerase/dNTP exclusion, dspacer, d block, ribostoper and annealing linear DNAs of different lengths. Methods for generating linkages with covalent attachments include, but are not limited to PCR and gene assembly techniques.

As used herein in the specification, “a” or “an” may mean one or more. As used herein, when used in conjunction with the word “comprising”, the words “a” or “an” may mean one or more than one. As used herein “another” may mean at least a second or more.

As used herein, “plurality” means more than one. In certain specific aspects, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any integer derivable therein, and any range derivable therein.

As used herein, “any integer derivable therein” means an integer between the numbers described in the specification, and “any range derivable therein” means any range selected from such numbers or integers.

As used herein, a “fragment” refers to a sequence having or having at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more, or any range between any of the points or any other integer between any of thes points, contiguous residues of the polypeptide sequences set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70 SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, but less than the full-length of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116; or nucleotides of the recited SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, and/or SEQ ID NO:115, but less than the full-length of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115. It is contemplated that the definition of “fragment” can be applied to amino acid and nucleic acid fragments.

As used herein, an “antigenic fragment” refers to a fragment, as defined above, that can elicit an immune response in an animal.

Reference to a sequence in an organism, such as a “herpesvirus sequence” refers to a segment of contiguous residues that is unique to that organism(s) or that constitutes a fragment (or full-length region(s)) found in that organism(s) (either amino acid or nucleic acid).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. RELI round 1 challenge assay results by symptoms readout. Herpes disease severity was scored for groups of mice immunized with one of the 12 tPA-fused sublibraries (T1 through T12) or one of the 12 UB fused sublibraries (U1 through U12). Day 7 post-infection is presented since this is the day before control animals began to die. All animals were visually inspected for a variety of disease parameters. Values were assigned for the disease symptom, with increasing numbers indicting a worse disease. Edema, abdominal swelling, scabbing and scar formation were scored as 3, blisters and swollen lymph nodes as 5, lesions and erythemia as 6, ulcers and gut poresis were scored as 7, hypothermia as 8, paralysis and neural infections as 10 and death or euthanasia as 20. The values were further modified depending on whether the effect was very mild (+2), mild (+3), moderate (+5), severe (+7) or very severe (+9). The mouse groups scored as positive are displayed as black bars. Vector=plasmid without an HSV insert. Error bars represent standard errors of the mean.

FIG. 2. RELI round 1 challenge assay results by lethality readout. Protection from death was evaluated by determining survival rates for groups of mice immunized with one of the 12 tPA-fused sublibraries (T1 through T12) or one of the 12 UB fused sublibraries (U1 through U12). The percentage of animals remaining alive on days 7 through 9 post-exposure are plotted. Negative control animals began to die on day 7, no further deaths were observed from day 9 through the end of the monitoring period (day 14). The mouse groups scored as positive are marked with astericks. Vector=plasmid without an HSV insert; NI=non-immunized.

FIG. 3 An illustration of the three-dimensional grid built virtually to array the individual components of the HSV-1 library. The planar dimensions of the grid were used to define multiplexed pools. These pools were used as genetic inocula for ELI testing.

FIGS. 4A and 4B. Lethality results from challenge-protection assays in a second round of RELI, from the (FIG. 4A) tPA and (FIG. 4B) UB fusion libraries. The library components comprising the positively scoring pools from the round 1 study were re-arrayed into new pools defined by the X, Y, and Z planes of a cube. These were assayed by genetic immunization alongside control inocula, which are displayed as gray bars. Vector=plasmid without insert; NI=no inoculum. The round 1 sublibraries selected for reduction were retested. RD1#1, RD1#3, and RD1#8 from the tPA screen and RD1#6 (Rd+) and RD1#11 (Rd+) from the UB screen. The mouse groups scored as positive are marked with astericks.

FIGS. 5A and 5B. Protection analyses of single plasmid clones reduced from the two HSV1 libraries. Sequencing of the library clones inferred from the matrix analyses of the round 2 data identified ORFs for testing in round 3. These were assayed by genetic immunization alongside control inocula, which are displayed as gray bars. pCMVigD=plasmid expressing the previously described HSV antigen, Irrel=a non-HSV library inoculum, NI=non-immunized. The UB library-derived clones were administered at a 200-fold diluted DNA-dose relative to that used for the tPA-derived clones. (FIG. 5A) For the round 3 testing from the tPA library, the percentage of mice alive on representative days 9, 12, 13, and 14 is presented. (FIG. 5B) For the round 3 testing from the UB library, days 8, 9, and 14 are plotted. Inocula scored as positive are marked with astericks.

FIGS. 6A and 6B. Comparative testing of the ORFs inferred from both the tPA and UB grids. Library clones were tested in parallel, at equivalent doses. (FIG. 6A) The survival rates of mice immunized with each candidate on representative days 8, 9, 10, 11, and 14. (FIG. 6B) The average survival scores for each of these inoculated groups of mice plotted. These calculated values integrate survival during the period from 8 to 14 days post-challenge.

FIG. 7A-7C. Survival rates from a directed-ELI study. Groups of mice were immunized with HSV-1 ORFs that had been pooled for three-dimensional matrix analyses. Each data set represents the (FIG. 7A) X, (FIG. 7B) Y, or (FIG. 7C) Z axis. Error bars represent standard errors of the mean.

FIG. 8A-8C. Average survival scores from a directed-ELI study. The same data presented above as percent survival on individual days was used to derive a single score representing extended survival during the monitoring period. Once the non-immunized began to die, the day-numbers that each mouse survived were summed. The sum for each animal per group was averaged to determine a group survival score. As in FIG. 1, each data set represents the (FIG. 8A) X, (FIG. 8B) Y, or (FIG. 8C) Z axes. Positively scored groups are shaded black. Positive and negative control groups are gray-shaded.

FIGS. 9A and 9B. Initial testing of individual ORFs inferred from the triangulation analysis of the DELI grid. Both the ORFs tested and their derivative genes are given. Protection is presented as (FIG. 9A) rates of extended survival on several representative days and as (FIG. 9B) survival scores, calculated from days 8 through 14 post-exposure. Groups displaying non-overlapping error bars with the non-immunized are shown in black. Positive and negative control groups are gray-shaded.

### DESCRIPTION OF ILLUSTRATIVE EMBODIMENTS

The present invention overcomes the current limitations of herpesvirus vaccines by providing isolated nucleic acids and/or polypeptides from one or more members of the Herpesvirus family (Herpesviridae) that are typically protective. Certain embodiments include isolated nucleic acids and/or polypeptides from Herpes Simplex Virus type 1 and type 2 (HSV-1 and HSV-2, respectively) or other herpesviruses (i.e., , VZV, BHV, EBV, CMV, CHV, or EHV). Compositions comprising isolated nucleic acids and polypeptides of a herpesvirus, as well as methods of using such compositions, may provide prophylactic or therapeutic immunization against members of the Herpesvirus family. By introduction of one or more of the compositions of the present invention, a subject may be induced to produce antibodies against one or more viruses of the Herpesvirus family, specifically the Alphaherpesvirus sub-family (Alphaherpesvirinae), which includes the closely related viruses HSV-1 and HSV-2. In other embodiments of the invention, binding agents such as antibodies, anticalins, and the like may be used in passive immunization or in other therapeutic modalities.

Widespread human infection by members of the Herpesvirus family represents a particular challenge for vaccinology. For example, herpesvirus infections in humans may lead to mononucleosis, blindness, encephalitis, cancer or other disease conditions. Thus, an effective treatment for herpesvirus infections in humans and other vertebrate animals is of clinical importance. In the present invention, the expression library immunization (ELI) process used both without, and also in combination with, LEEs may be utilized to identify vaccine candidates against herpesvirus infections and associated diseases. Clinically, some of the goals of treatment for or immunization against herpesviruses may include reducing the severity of disease associated with primary infection; reducing the frequency of reactivation of latent virus; limiting the severity of reactivated disease; and restricting the transmission of virus associated with either primary or reactivated infection(s).

A comprehensive, unbiased approach to antigen selection for a subunit vaccine is enabled by combining genetic immunization (Tang et al., 1992) with the invention of expression library immunization (ELI) (Barry et al., 1995). ELI is an empirical method, as was Jenner's, to identify protective vaccines. However, unlike Jenner's it is based on a subunit rather than whole pathogen endproduct. Using ELI, the entire genome of a pathogen can be searched for protective antigens. Pathogen DNA is fragmented and cloned into a mammalian expression vector to generate a library corresponding to all of the genetic material of the organism. In 1995 the utility of ELI was demonstrated in the protection of mice against Mycoplasma (M.) pulmonis challenge by prior vaccination with a pathogen library. The complete library is partitioned into sub-libraries that are used to separately immunize groups of test animals. Sub-library inocula that protect animals from disease following challenge are scored as positive. Presumably one or more plasmids within a positive sub-library are responsible for the protective response. To identify the constituent antigen-expressing plasmid(s) that holds protective capacity, the sub-libraries can be further subdivided and tested. Plasmid DNA is prepared from the pools and used to inoculate more test animals, which are assayed for protection. Other researchers have subsequently reported the successful application of ELI against other bacterial and parasitic pathogens. Brayton et. al. used a Rickettsia (Cowdria ruminantium) expression library to screen for protective sub-library pools in a murine model of Heartwater disease. Four out of ten groups of mice inoculated with different sub-libraries and challenged with an optimal level of bacteria showed reduced levels of infection (Brayton et al., 1998). In another study, a partial expression library was made from cDNA of the parasitic helminth Taenia crassiceps and used to immunize mice against cysticerosis disease. Though the inoculum only represented a portion of the genome, a two-fold reduction in parasitemia was observed (Manoutcharian et al., 1998). Alberti et. al. found that an expression library made from the genome of Trypanosoma cruzi (a protozoa that causes Chagas' disease) stimulated specific immune responses in mice (Alberti et al., 1998). A library made from the genomic DNA of Leishmania major (a protozoan that causes leismaniasis) was able to marginally reduce parasite load in challenged mice (Piedrafita et al., 1999). Test mice inoculated with further sub-divisions of this library displayed greater levels of protection than the original. This indicates that the protective clone(s) was being enriched through two rounds of reduction in the complexity of the plasmid inocula. In a recent study, random genomic DNA fragments from Mycoplasma hyopneumoniae were cloned into an expression vector, screened for open-reading frames, and then used to immunize pigs. These libraries were shown to protect this natural pathogen host from infection (Moore et al., 2001). In addition, Smooker et al. (2000) have studied ELI in the context of immunization of rodents against Malaria.

The ELI studies presented to date have shown that mixed antigen libraries can protect against disease, and in some cases the complexities of the original mixtures have been reduced. ELI as originally presented, with random-fragment plasmid-clones (RELI) is capable of providing effective vaccine candidates. However, we have also dramatically improved ELI so as to yield many more vaccine candidates and with much less time and technical difficulty. The availability of sequenced pathogen genomes enables sequence-directed primers to be designed and ORFs to be amplified by PCR. Since each library member is defined, complete genomic coverage is ensured and constructs can be placed in position for proper expression. This eliminates a statistically invoked redundancy that was necessary, and consequently directed-ELI (DELI) reduces the library sizes and number of sibbing rounds. The technical challenge for practicing directed-ELI was constructing enough individual library clones to represent all ORFs of the genome. To avoid the formidable task of thousands of cloning steps linear expression elements (LEEs) were developed. In an LEE protocol, PCR-amplified ORFs can be linked to a desired promoter and a terminator, and then directly delivered into animals for gene expression.

The present invention provides compositions and methods for the immunization of vertebrate animals, including humans, against herpesvirus infections. Compositions of the invention may comprise isolated nucleic acids encoding herpesvirus polypeptide(s); herpesvirus polypeptides, including complements, fragments, mimetics or closely related sequences, as antigenic components; and/or binding or affinity agents that bind antigens derived from herpesvirus members. Identification of the nucleic acids and polypeptides of the invention is typically carried out by adapting ELI and LEE methodology to screen a herpesvirus genome(s) (e.g., an HSV-1 genome) for vaccine candidates. The compositions and methods of the invention may be useful for vaccination against herpesvirus infections (e.g., HSV-1 and HSV-2 infections).

In various embodiments, a vaccine composition directed against a member of the Herpesvirus family may be provided. The vaccine according to the present invention may comprise a herpesvirus nucleic acid(s) and/or polypeptide(s). In particular embodiments, the herpesvirus is a HSV virus, preferably HSV-1 or HSV-2. The vaccine compositions of the invention may confer protective or therapeutic resistance to a subject against HSV and/or other herpesvirus infections.

In still other embodiments, the invention may provide screening methods that include constructing an expression library via LEEs and screening it by expression library immunization in order to identify herpesvirus genes (e.g., HSV-1 genes) that confer protection against or therapy for herpesvirus infection. Additionally, methods may be used to identify and utilize polynucleotides and polypeptides derived from other related organism or by synthesizing a molecule that mimics the polypeptides of identified herpesvirus polypeptides.

### I. Herpesviridae

Members of the Herpesvirus family (Herpesviridae) replicate in the nucleus of a wide range of vertebrate hosts, including eight species isolated in humans, several each in horses, cattle, mice, pigs, chickens, turtles, lizards, fish, and even in some invertebrates, such as oysters. Human herpesvirus infections are endemic and sexual contact is a common method of transmission for several of the viruses including both herpes simplex virus 1 and 2 (HSV-1, HSV-2). The increasing prevalence of genital herpes and corresponding rise of neonatal infection and the implication of Epstein-Barr virus (EBV or HHV-4) and Kaposi's sarcoma herpesvirus as cofactors in human cancers create an urgency for a better vaccination against this virus family.

All herpesvirus virions have an envelope, a capsid, a tegument, and a core. The core includes a single linear molecule of dsDNA. The capsid surrounds the core and is an icosahedron of approximately 100 nm in diameter. The capsid is constructed of 162 capsomeres consisting of 12 pentavalent capsomers (one at each apex) and 150 hexavalent capsomers. The tegument is located between the capsid and the envelope. The tegument is an amorphous, sometimes asymmetrical, feature of the Herpesvirus family. It consists of viral enzymes, some of which are needed to take control of a host cell's chemical processes and subvert them to virion production, some of which defend against the host cell's immediate responses, and others for which the function is not yet understood. The envelope is the outer layer of the virion and is composed of altered host membrane and a dozen unique viral glycoproteins, which appear in electron micrographs as short spikes embedded in the envelope.

Herpesvirus genomes range in length from 120 to 230 kilobasepairs (kbp) with base composition from 31% to 75% G+C content and contain 60 to 120 genes. Because replication takes place inside the nucleus, herpesviruses can use both the host's transcription machinery and DNA repair enzymes to support a large genome with complex arrays of genes. Herpesvirus genes are not arranged in operons and in most cases have individual promoters. However, unlike eukaryotic genes, very few herpesvirus genes are spliced. All herpesvirus genomes contain lengthy terminal repeats both direct and inverted. There are six terminal repeat arrangements and understanding how these repeats function in viral success is not completely understood.

The Herpesvirus family is generally divided into three sub-families, Alphaherpesvirinae, Betaherpesvirinae, and Gammaherpesvirinae. The Alphaherpesvirus sub-family includes the Simplexviruses (e.g., HSV-1 and HSV2) and the Varicellovirus (e.g., Varicella Zoster Virus, VZV). The Betaherpesvirus sub-family includes Cytomegalovirus (e.g., human herpesvirus 5 (HHV-5) or CMV), Muromegalovirus (e.g., mouse cytomegalovirus 1), and Roseolovirus (e.g. HHV-6 and HHV-7). Finally, the Gammaherpesvirus sub-family includes Lymphocryptovirus (e.g., HHV-4 or EBV) and Rhadinovirus (e.g., HHV-8). A more detailed review of the Herpesvirus Family may be found in Fields Virology (1996), which is incorporated herein by reference.

### II. Vaccines

The concept of vaccination/immunization is based on two fundamental characteristics of the immune system, namely specificity and memory of immune system components. Vaccination/immunization will initiate a response specifically directed to the antigen with which a subject was challenged. Furthermore, a population of memory B and T lymphocytes may be induced. Upon re-exposure to the antigen(s) or the pathogen an antigen(s) was derived from, the immune system will be primed to respond much faster and much more vigorously, thus endowing the vaccinated/immunized subject with immunological protection against a pathogen or disease state. Protection may be augmented by administration of the same or different antigen repeatedly to a subject or by boosting a subject with a vaccine composition.

Vaccination is the artificial induction of actively-acquired immunity by administration of all or part of a non-pathogenic form or a mimetic of a disease-causing agent. The aim is to prevent a disease or treat a symptom of a disease, so the procedure may also be referred to as prophylactic or therapeutic immunization, respectively. In addition to actively-acquired immunity, passive immunization methods may also be used to provide a therapeutic benefit to a subject, see below.

In particular, genetic vaccination, also known as DNA immunization, involves administering an antigen-encoding expression vector(s) in vivo, in vitro, or ex vivo to induce the production of a correctly folded antigen(s) within an appropriate organism, tissue, cell or a target cell(s). The introduction of the genetic vaccine will cause an antigen to be expressed within those cells, an antigen typically being part or all of one or more protein or proteins of a pathogen. The processed proteins will typically be displayed on the cellular surface of the transfected cells in conjunction with the Major Histocompatibility Complex (MHC) antigens of the normal cell. The display of these antigenic determinants in association with the MHC antigens is intended to elicit the proliferation of cytotoxic T-lymphocyte clones specific to the determinants. Furthermore, the proteins released by the expressing transfected cells can also be picked up, internalized, or expressed by antigen-presenting cells to trigger a systemic humoral antibody responses.

A vaccine is a composition including an antigen derived from all or part of a pathogenic agent, or a mimetic thereof that is modified to make it non-pathogenic and suitable for use in vaccination. The term vaccine is derived from Jenner's original vaccine that used cowpoxvirus isolated from cows to immunize humans against smallpox. Vaccines may include polynucleotides, polypeptides, attenuated pathogens, killed (or inactivated) pathogens, inactivated toxins, mimetics of an antigen and/or other antigenic materials that induce an immune response in a subject. These antigens may be presented in various ways to the subject being immunized or treated. Types of vaccines include, but are not limited to genetic vaccines, virosomes, attenuated or inactivated whole organism vaccines, recombinant protein vaccines, conjugate vaccines, transgenic plant vaccines, toxoid vaccines, purified sub-unit vaccines, multiple genetically-engineered vaccines, anti-idiotype vaccines, peptide mimetopes and other vaccine types known in the art.

An immune response may be an active or a passive immune response. Active immunity develops when the body is exposed to various antigens. It typically involves B or T lymphocytes. B lymphocytes (also called B cells) produce antibodies. Antibodies attach to a specific antigen and make it easier for phagocytes to destroy the antigen. Typically, T lymphocytes (T cells) help B cells make antibodies and other T cells attack antigens directly or kill virus infected cells and may provide some control over the infection. B cells and T cells develop that are specific for a particular antigen or antigen type. Passive immunization generally refers to the administration of preformed antibodies or other binding agents, which bind an antigen(s). One of the various goals of immunization is to provide a certain protection against or treatment for an infection or disease associated with an infection or the presence of a pathogen.

In certain cases, an immune response may be a result of adoptive immunotherapy. In adoptive immunotherapy, lymphocyte(s) are obtained from a subject and are exposed or pulsed with an antigenic composition in vitro, and then administered back to the subject. The antigenic composition may comprise additional immunostimulatory agents or a nucleic acid encoding such agents, as well as adjuvants or excipients, see below. In certain instances, lymphocyte(s) may be obtained from the blood or other tissues of a subject. Lymphocyte(s) may be peripheral blood lymphocyte(s) and may be administered to the same or different subjects, referred to as autologous or heterologous donors respectively (for exemplary methods or compositions see U.S. Pat. Nos. 5,614,610; 5,766,588; 5,776,451; 5,814,295; 6,004,807 and 6,210,963).

The present invention includes methods of immunizing, treating or vaccinating a subject by contacting the subject with an antigenic composition comprising a herpesvirus antigen or antigens or a polynucleotide(s) encoding a herpesvirus antigen or antigens. An antigenic composition may comprise a nucleic acid; a polypeptide; an attenuated pathogen, such as a virus, a bacterium, a fungus, or a parasite, which may or may not express a herpesvirus antigen; a prokaryotic cell expressing a herpesvirus antigen; a eukaryotic cell expressing a herpesvirus antigen; a virosome; and the like, or a combination thereof. As used herein, an “antigenic composition” will typically comprise an antigen in a pharmaceutically acceptable formulation.

Antigen refers to any substance, molecule, or molecule encoding a substance that a host regards as foreign and therefore elicits an immune response, particularly in the form of specific antibodies or T-cells reactive to an antigen. An antigenic composition may further comprise an adjuvant, an immunomodulator, a vaccine vehicle, and/or other excipients, as described herein and is known in the art (for example see Remington's Pharmaceutical Sciences).

A herpesvirus antigen is an antigen that is derived from any virus that is a member of the Herpesvirus family. In particular embodiments a herpesvirus antigen may be an antigen derived from a HSV-1 or HSV-2 virus.

Various methods of introducing an antigen or an antigen composition to a subject are known in the art. Vaccination methods include, but are not limited to DNA vaccination or genetic immunization (for examples see U.S. Pat. Nos. 5,589,466, 5,593,972, 6,248,565, 6,339,086, 6,348,449, 6,348,450, 6,359,054, each of which is incorporated herein by reference), edible transgenic plant vaccines (for examples see U.S. Pat. Nos. 5,484,719, 5,612,487, 5,914,123, 6,034,298, 6,136,320, and 6,194,560, each of which is incorporated herein by reference), transcutaneous immunization (Glenn et al., 1999 and U.S. Pat. No. 5,980,898, each of which is incorporated herein by reference), nasal or mucosal immunization (for examples see U.S. Pat. Nos. 4,512,972, 5,429,599, 5,707,644, 5,942,242, each of which is incorporated herein by reference); virosomes (Huang et al., 1979; Hosaka et al., 1983; Kaneda, 2000; U.S. Pat. Nos. 4,148,876; 4,406,885; 4826,687; 5,565,203; 5,910,306; 5,985,318, each of which is incorporated herein by reference), live vector and the like. Antigen delivery methods may also be combined with one or more vaccination regimes.

Vaccines comprising an antigen, a polypeptide or a polynucleotide encoding an antigen may present an antigen in a variety of contexts for the stimulation of an immune response. Some of the various vaccine contexts include attenuated pathogens, inactivated pathogens, toxoids, conjugates, recombinant vectors, and the like. Many of these vaccines may contain a mixture of antigens derived from the same or different pathogens. Polypeptides of the invention may be mixed with, expressed by or couple to various vaccine compositions. Various vaccine compositions may provide an antigen directly or deliver an antigen producing composition, e.g., an expression construct, to a cell that subsequently produces or expresses an antigen or antigen encoding molecule.

A. Genetic Vaccines

Immunization against an antigen or a pathogen may be carried out by inoculating, transfecting, or transducing a cell, a tissue, an organ, or a subject with a nucleic acid encoding an antigen. One or more cells of a subject may then express the antigen encoded by the nucleic acid. Thus, the antigen encoding nucleic acids may comprise a “genetic vaccine” useful for vaccination and immunization of a subject. Expression in vivo of the nucleic acid may be, for example, from a plasmid type vector, a viral vector, a viral/plasmid construct vector, or an LEE or CEE construct.

In preferred aspects, the nucleic acid comprises a coding region that encodes all or part of an antigenic protein or peptide, or an immunologically functional equivalent thereof. Of course, the nucleic acid may comprise and/or encode additional sequences, including but not limited to those comprising one or more immunomodulators or adjuvants. A nucleic acid may be expressed in an in vivo, ex vivo or in vitro context, and in certain embodiments the nucleic acid comprises a vector for in vivo replication and/or expression. For exemplary compositions and methods see U.S. Pat. Nos. 5,589,466; 6,200,959; and 6,339,068; each of which is incorporated herein by reference.

B. Polypeptide Vaccines

In accordance with the present invention, one may utilize antigen compositions containing one or more antigenic polypeptide(s), as well as variants or mimics thereof, to induce an immune response in a subject. Antigenic polypeptides of the invention may be synthesized or purified from a natural or recombinant source and used as a component of a polypeptide vaccine. In various embodiments, polypeptides may include fusion proteins, isolated polypeptides, polypeptides conjugated with other immunogenic molecules or substances, polypeptide mixtures with other immunogenic molecules or substances, and the like (for exemplary methods and/or compositions see U.S. Pat. Nos. 5,976,544; 5,747,526; 5,725,863; and 5,578,453; each of which is incorporated herein by reference).

C. Purified Sub-Unit Vaccines

Compositions and methods described herein may be used to isolate a portion of a pathogen for use as a sub-unit vaccine. Sub-unit vaccines may utilize a partially or substantially purified molecule of a pathogen as an antigen. Polynucleotides and/or polypeptides of the invention may serve as a sub-unit vaccine or be used in combination with or be included in a sub-unit vaccine for herpesvirus. Methods of sub-unit vaccine preparation may include the extraction of certain antigenic molecules from a bacteria, virus, parasite and/or other pathogens by known purification methods. The preparation of a sub-unit vaccine may neutralize the pathogenicity of an entire pathogen rendering the vaccine, itself, non-infectious. Examples include influenza vaccine (viral surface hemagglutinin molecule) and the Neisseria meningitidis vaccine (capsular polysaccharide molecules). Advantages include high purity, only rare adverse reaction and highly specific immunity. Protein sub-units may be produced in non-pathogenic microbes by genetic engineering techniques making production much safer.

D. Conjugate Vaccines

The compositions and antigens of the invention may be conjugated to other molecules to produce a conjugate vaccine. Polysaccharides found to be poorly immunogenic by themselves have been shown to be quite good immunogens once they are conjugated to an immunogenic protein (U.S. Pat. No. 4,695,624, incorporated herein by reference). Conjugate vaccines may also be used to enhance the immunogenicity of an antigenic polypeptide. Conjugate vaccines utilize the immunologic properties of certain peptides to enhance the immunologic properties of glycolipids, polysaccharides, other polypeptides and the like. Certain embodiments of the invention contemplate using conjugates to enhance the immunogenicity of the polynucleotides and polypeptides of the invention. Examples of conjugate vaccines can be found in U.S. Pat. Nos. 6,309,646; 6,299,881; 6,248,334; 6,207,157; and 5,623,057; each of which is incorporated herein by reference.

E. Virus-like Particle (VLP) Vaccines

Polynucleotides and polypeptides of the invention may be used in conjunction with VLP vaccines. In many virus species, virus proteins are capable of assembling in the absence of nucleic acid to form so-called virus-like particles or VLPs. Similarly, the proteins which normally cooperate together with nucleic acid to form the virus core can assemble in the absence of nucleic acid to form so-called core-like particles (CLPs). The terms “virus-like particles” and “core-like particles” will be used to designate assemblages of virus proteins (or modified or chimeric virus proteins) in the absence of a viral genome. The addition of antigenic peptide in the context of these particles may be especially useful in the development of vaccines for oral or other mucosal routes of administration (for examples see U.S. Pat. No. 5,667,782, which is hereby incorporated by reference). In other embodiments of the invention a virosome also may be used. Examples of virosome compositions and methodology can be found in U.S. Pat. Nos. 4,148,876; 4,406,885; 4,826,687; and Kaneda, 2000, each of which is incorporated herein by reference.

F. Cell Mediated Vaccines

An alternative method of presenting antigens is to use genetically modified cells as an expression or delivery vehicle for polynucleotides or polypeptides of the invention. For example, cells may be isolated from a subject or another donor and transformed with a genetic construct that expresses an antigen, as described herein. Following selection, antigen-expressing cells are cultured as needed. The cells may then be introduced or reintroduced to a subject, where these cells express an antigen and induce an immune response (see U.S. Pat. Nos. 6,228,640; 5,976,546; and 5,891,432, each of which is incorporated herein by reference).

In certain embodiments, cell mediated vaccines may include vaccines comprising antigen presenting cells (APC). A cell that displays or presents an antigen normally or preferentially with a class II major histocompatibility molecule or complex to an immune cell is an “antigen presenting cell.” Secreted or soluble molecules, such as for example, cytokines and adjuvants, may also aid or enhance the immune response against an antigen. Such molecules are well known to one of skill in the art, and various examples are described herein.

The dendritic cell (DC) is a cell type that may be used for cell-mediated vaccination, as they are potent antigen presenting cells, effective in the stimulation of both primary and secondary immune responses (Steinman, 1999; Celluzzi and Falo, 1997). It is contemplated in the present invention that the exposure or transformation of dendritic cells to an antigenic composition of the invention, will typically elicit a potent immune response specific for a virus of the Herpesvirus family, e.g. HSV-1 or HSV-2. In particular embodiments an antigen may be reacted or coated with antibodies prior to presentation to an APC.

G. Edible Vaccines

An edible vaccine is a food plant or food-stuff that is used in delivering an antigen that is protective against an infectious disease, a pathogen, an organism, a bacterium, a virus or a non-infectious disease such as an autoimmune disease. In particular, the invention provides for an edible vaccine that induces a state of immunization against a member of the Herpesvirus family. The present invention may also include gene constructs or chimeric gene constructs comprising a coding sequence of at least one of the polypeptides, peptides, or fragments thereof of the invention, plant cells and transgenic plants transformed with said gene constructs or chimeric gene constructs, and methods of preparing an edible vaccine from these plant cells and transgenic plants. For exemplary methods see U.S. Patent publication 20020055618 and U.S. Pat. Nos. 5,914,123; 6,034,298; 6,136,320; 6,444,805; and 6,395,964, which are incorporated herein by reference. The present invention also provides methods of treating disease or infection with edible vaccines and compositions comprising edible vaccines according to the invention.

Numerous plants may be useful for the production of an edible vaccine, including: tobacco, tomato, potato, eggplant, pepino, yam, soybean, pea, sugar beet, lettuce, bell pepper, celery, carrot, asparagus, onion, grapevine, muskmelon, strawberry, rice, sunflower, rapeseed/canola, wheat, oats, maize, cotton, walnut, spruce/conifer, poplar and apple. An edible vaccine may include a plant cell transformed with a nucleic acid construct comprising a promoter and a sequence encoding a peptide of the invention. The sequence may optionally encode a chimeric protein, comprising, for example, a cholera toxin subunit B peptide fused to the peptide. Plant promoters of the invention include, but are not limited to CaMV 35S, patatin, mas, and granule-bound starch synthase promoters. Additional useful promoters and enhancers are described in WO 99/54452, incorporated herein by reference.

The edible vaccine of the invention can be administered to a mammal suffering from or at risk of disease or infection. Preferably, an edible vaccine is administered orally, e.g. consuming a transgenic plant of the invention. The transgenic plant can be in the form of a plant part, extract, juice, liquid, powder, or tablet. The edible vaccine can also be administered via an intranasal route.

H. Live Vector Vaccines

In another embodiment, a live vector vaccine may be prepared comprising attenuated and/or non-pathogenic micro-organisms, e.g. viruses or bacteria containing polynucleotides or nucleic acids encoding the peptides or antigens of the present invention expressed in the same or different micro-organisms. Live vector vaccines, also called “carrier vaccines” and “live antigen delivery systems”, comprise an exciting and versatile area of vaccinology (Levine et al, 1990; Morris et al., 1992; Barletta et al., 1990; Dougan et al., 1987; and Curtiss et al., 1989; U.S. Pat. Nos. 5,783,196; 5,648,081; and 6,413,768; each of which is incorporated herein by reference). In this approach, a live viral or bacterial vaccine is modified so that it expresses protective foreign antigens of another microorganism, and delivers those antigens to the immune system, thereby stimulating a protective immune response. Live bacterial vectors that are being promulgated include, among others, attenuated Salmonella (Levine et al., 1990; Morris et al., 1992; Dougan et al., 1987; and Curtiss et al., 1989), Bacille Calmette Guerin (Barletta et al., 1990), Yersinia enterocolitica (Van Damme et al., 1992), V. cholerae O1 (Viret et al., 1993)) and E. coli (Hale, 1990). The use of attenuated organisms as live vectors/vaccines expressing protective antigens of relevant pathogens is well-known.

I. Attenuated Pathogen Vaccines

In certain embodiments, a herpesvirus antigen may be incorporated in or coupled to an attenuated pathogen or cell, which may encode, express, or is coupled to the antigen. Attenuation may be accomplished by genetic engineering, altering pathogen culture conditions, treatment of the pathogen, such as chemical or heat inactivation or other means. The antigen encoded by an attenuated pathogen is one which when expressed or exposed is capable of inducing an immune response and providing protection and/or therapy in an animal or human against a virus from one or more members of the Herpesvirus family from which the antigen was derived, or from a related organism. Herpesvirus antigens may be introduced into an attenuated pathogen by way of DNA encoding the same. For exemplary methods and compositions see U.S. Pat. Nos. 5,922,326; 5,922,326; 5,607,852 and 6,180,110.

J. Killed Pathogen Vaccines

An antigen may also be associated with a killed or inactivated pathogen or cell. Killed pathogen vaccines include preparations of wild-type pathogens, or a closely-related pathogen, that has been treated to make them non-viable (inactivated). Methods of inactivation include heat-killing of a pathogen. One advantage of heat killing is that it leaves no extraneous residue, but may alter protein conformations and hence immunogenic specificity, however it is useful for vaccines in which the immunogenic molecule is a polysaccharide. Alternative methods of killing include chemicals (β-propio-lacone or formaldehyde), which may leave a toxic residue, but does not alter protein conformations significantly and preserves immunogenic specificity. Killed pathogen vaccines may be use in combination with other vaccine vehicles as described herein. For exemplary methods and compositions see U.S. Pat. Nos. 6,303,130, 6,254,873, 6,129,920 and 5,523,088, each of which is incorporated herein by reference.

K. Humanized Antibodies

Polypeptides, fragments or mimetics thereof, of the invention may be used to produce anti-idiotypic antibodies for use in a vaccine. In an anti-idiotype vaccine the immunogen is an antibody against the Fab end of a second antibody which was raised against an antigenic molecule of a pathogen. The Fab end of the anti-idiotype antibody will have the same antigenic shape as the antigenic molecule of the pathogen and may then be used as an antigen (see exemplary U.S. Pat. Nos. 5,614,610 and 5,766,588). “Humanized” antibodies for use herein may be antibodies from non-human species wherein one or more selected amino acids have been exchanged for amino acids more commonly observed in human antibodies. This can be readily achieved through the use of routine recombinant technology, particularly site-specific mutagenesis. Humanized antibodies may also be used as a passive immunization agent as described below.

### III. Antigen Screening Methods

Methods of screening for at least one test polypeptide or test polynucleotide encoding a polypeptide for an ability to produce an immune response may comprise (i) obtaining at least one test polypeptide or test polynucleotide by (a) amplifying the polynucleotide by PCR; (b) building the polynucleotide by gene assembly; (c) modifying the amino acid sequence of a known antigenic polypeptide or polynucleotide sequence of a polynucleotide encoding a known antigenic polypeptide; (d) obtaining a homolog of a known antigenic sequence of a polynucleotide encoding such a homolog, or (e) obtaining a homolog of a known antigenic sequence or a polynucleotide encoding such a homolog and modifying the amino acid sequence of the homolog or the polynucleotide sequence of the polynucleotide encoding such a homolog; and (ii) testing the test polypeptide or test polynucleotide under appropriate conditions to determine whether the test polypeptide is antigenic or the test polynucleotide encodes an antigenic polypeptide.

A method of screening may include identifying a polypeptide by testing mixtures of linear polynucleotides that encode a polypeptide for protection against disease or infection.

A method of screening may include obtaining a test polypeptide by modifying the amino acid sequence or obtaining a homolog of a least one polypeptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116 or fragment thereof. The method of screening may also include a test polypeptide comprising an amino acid sequence of at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116 or fragment thereof, which has been modified.

In other embodiments the method of screening may also include obtaining a test polynucleotide comprising a polynucleotide encoding a modified amino acid sequence of or a homolog of at least one polypeptide having a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116 or fragment thereof or obtaining a test polynucleotide comprising modifying the polynucleotide sequence of at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115 or fragment thereof. In various embodiments a method of screening may further comprise identifying at least one test polypeptide as being antigenic or at least one test polynucleotide as encoding an antigenic polypeptide.

The methods described may include placing an identified antigenic polypeptide or the polynucleotide encoding an antigenic polypeptide in a pharmaceutical composition. The methods may also include using an identified antigenic polypeptide or polynucleotide encoding an antigenic polypeptide to vaccinate a subject. In certain aspects a subject may be vaccinated against a herpesvirus and in particular HSV-1. Additionally, the subject may be vaccinated against a non-herpesvirus disease.

Additional embodiments include a method of preparing a vaccine including obtaining an antigenic polypeptide or a polynucleotide encoding an antigenic polypeptide, as determined to be antigenic by known screening methods and/or screening methods described herein, and placing a polypeptide or a polynucleotide in a vaccine composition. A vaccine composition may be used in vaccinating a subject by preparing a vaccine as described and vaccinating a subject with the vaccine.

### IV. Herpesvirus Antigens

Antigens of the invention are typically isolated from members of Herpesvirus family, in particular the Alphaherperviruses, namely HSV-1, HSV-2, VZV, and BHV. In particular embodiments, the immunization of vertebrate animals according to the present invention includes a library of herpesvirus coding sequences in expression constructs. In various embodiments, a DNA expression construct may be in the context of a linear expression elements (“LEEs”) and/or circular expression elements (“CEEs”), which typically encompass a complete gene (promoter, coding sequence, and terminator). These LEEs and CEEs can be directly introduced into and expressed in cells or an intact organism to yield expression levels comparable to those from a standard supercoiled, replicative plasmid (Sykes and Johnston, 1999). In specific embodiments, an expression library of HSV (e.g., HSV-1 and HSV-2) is provided. Expression library immunization, ELI herein, is well known in the art (U.S. Pat. No. 5,703,057, specifically incorporated herein by reference). In certain embodiments, the invention provides an ELI method applicable to virtually any pathogen and requires no knowledge of the biological properties of the pathogen. The method operates on the assumption, generally accepted by those skilled in the art, that all the possible polypeptide-based determinants of any pathogen are encoded in its genome. The inventors have previously devised methods of identifying vaccines using a genomic expression library representing all of the polypeptide-based determinants of a pathogen (U.S. Pat. No. 5,703,057). The method uses to its advantage the simplicity of genetic immunization to sort through a genome for immunological reagents in an unbiased, systematic fashion.

The preparation of an expression library is performed using the techniques and methods familiar to one of skill in the art (Sambrook et al., 2001). The pathogen's genomic sequence, may or may not be known. Thus one obtains DNA (or cDNA), representing substantially the entire genome of the pathogen (e.g., HSV-1). The DNA is broken up, by physical fragmentation or restriction endonuclease, into segments of some length so as to provide a library of about 10⁵ (approximately 18× the genome size) members. The library is then tested by inoculating a subject with purified DNA of the library or sub-library and the subject challenged with a pathogen, wherein immune protection of the subject from pathogen challenge indicates a clone that confers a protective immune response against infection.

In some embodiments of the invention, a herpesvirus antigen may be obtained by methods comprising: (a) preparing a sequence-directed linear expression element library prepared from nucleic acids (e.g., genomic DNA) of a member of the Herpesvirus family; (b) administering at least one LEE of the library in a pharmaceutically acceptable carrier into an animal; and (c) expressing at least one herpesvirus antigen in the animal. The expression library may comprise at least one or more polynucleotides having a sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115; a complement, a fragment, or a closely related sequences thereof. The polynucleotides of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:35, SEQ ID NO:37, SEQ ID NO:41, SEQ ID NO:45, SEQ ID NO:49, SEQ ID NO:53, SEQ ID NO:57, SEQ ID NO:59, SEQ ID NO:61, SEQ ID NO:65, SEQ ID NO:69, SEQ ID NO:73, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, and SEQ ID NO:113 represent exemplary gene fragments identified using ELI and related technology, as described herein. In addition, polynucleotides of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:39, SEQ ID NO:39, SEQ ID NO:43, SEQ ID NO:47, SEQ ID NO:51, SEQ ID NO:55, SEQ ID NO:63, SEQ ID NO:63, SEQ ID NO:67, SEQ ID NO:71, SEQ ID NO:77, SEQ ID NO:81, SEQ ID NO:85, SEQ ID NO:89, SEQ ID NO:93, SEQ ID NO:97, SEQ ID NO:101, SEQ ID NO:105, and SEQ ID NO:115 are representative of exemplary full length gene sequences identified using ELI and related technologies, as described herein. The expression library may be cloned in a genetic immunization vector or any other suitable expression construct. The construct may comprise a gene encoding a mouse ubiquitin polypeptide positioned such that it produces a herpesvirus/mouse ubiquitin/antigen fusion protein designed to link the expression library polynucleotides to the ubiquitin gene. The vector may comprise a promoter operable in eukaryotic cells, for example a CMV promoter, or any other suitable promoter. In such methods, the polynucleotide may be administered by an intramuscular injection, intradermal injection, or epidermal injection or particle bombardment. The polynucleotide may likewise be administered by intravenous, subcutaneous, intralesional, intraperitoneal, oral, other mucosal, or inhaled routes of administration. In some specific, exemplary embodiments, the administration may be via epidermal injection/bombardment of at least 0.0025 μg to 5.0 μg of the polynucleotide. Administration may also be via intramuscular injection of at least 0.1 μg to 50 μg of the polynucleotide. In some cases, a second administration, for example, an intramuscular injection and/or epidermal injection, may be administered at least about two weeks or longer after the first administration. In these methods, the polynucleotide may be, but need not be, cloned into a viral expression vector, for example, a viral expression vector, including adenovirus, herpes-simplex virus, retrovirus or adeno-associated virus vectors. The polynucleotide may also be administered in any other method disclosed herein or known to those of skill in the art.

In still other embodiments, a herpesvirus antigen(s) maybe obtained by methods comprising: (a) preparing a pharmaceutical composition comprising at least one polynucleotide encoding an Herpesvirus antigen or fragment thereof; (b) administering one or more ORFs of the library in a pharmaceutically acceptable carrier into an animal; and (c) expressing one or more Herpesvirus antigens in the animal. The one or more polynucleotides can be comprised in one or more expression vectors.

Alternatively, methods of obtaining Herpesvirus antigen(s) may comprise: (a) preparing a pharmaceutical composition of at least one Herpesvirus antigen or an antigenic fragment thereof; and (b) administering the at least one antigen or fragment into an animal. The antigen(s) may be administered by an intramuscular injection, intravenous injection, subcutaneous injection, intradermal injection, epidermal injection, by inhalation, oral, or other mucosal routes.

Also described herein, are methods of obtaining polynucleotide sequences effective for generating an immune response against members of the Herpesvirus family, in particular HSV-1, in a non-human animal comprising: (a) preparing an expression library from genomic DNA of a virus selected from the Herpesvirus family; (b) administering one or more components of the library in a pharmaceutically acceptable carrier into the animal in an amount effective to induce an immune response; and (c) selecting from the library the polynucleotide sequences that induce an immune response, wherein the immune response in the animal is protective against herpesvirus infection. Such methods may further comprise testing the animal for immune resistance against a herpesvirus infection by challenging the animal with herpesvirus. In some cases, the genomic DNA has been fragmented physically or by restriction enzymes. DNA fragments may be, on average, about 300-1500 base pairs in length. In some cases, each component in the library may comprise a sequence encoding a mouse ubiquitin fusion polypeptide designed to link the expression library polynucleotides to the ubiquitin gene, but this is not required in all cases. In some cases, the library may comprise about 4 to about 400 or more ORFs; in more specific cases, the library could have 1×10⁵ ORFs. In some preferred methods, about 0.01 μg to about 5 μg of DNA, of the open-reading frames is administered into the animal. In some situations the genomic DNA, gene or cDNA is introduced by intramuscular injection or epidermal injection. In some versions of these protocols, the expression library further comprises a promoter operably linked to the DNA that permits expression in a vertebrate animal cell.

The application also discloses methods of preparing antigens that confer protection against infection in a vertebrate animal comprising the steps of: (a) preparing an ORF expression library from PCR-amplified genomic DNA of a herpes simplex virus; (b) administering one or more ORFs of the library in a pharmaceutically acceptable carrier into the animal in an amount effective to induce an immune response; (c) selecting from the library the polynucleotide sequences that induce an immune response (d) expressing the polynucleotide sequences in cell culture, such as a eukaryotic or prokaryotic expression system; and (e) purifying the polypeptide(s) expressed in the cell culture. Often, these methods further comprise testing the animal for immune resistance against infection by challenging the animal with one or more herpesvirus or other pathogens.

In yet other embodiments the invention relates to methods of preparing antibodies against a herpesvirus antigen comprising the steps of: (a) identifying an HSV antigen that confers immune resistance against an infection of HSV or other member of the family when challenged with a selected member of the Herpesvirus family; (b) generating an immune response in a vertebrate animal with the antigen identified in step (a); and (c) obtaining antibodies produced in the animal.

The invention also relates to methods of preparing antibodies against a herpesvirus polypeptide that is immunogenic, but not necessarily protective as a vaccine. For example herpes-specific antibodies might be useful in research analyses, diagnosis or antibody-therapy. Immunizing animals with the identified antigen might produce antibodies, or expressing the gene encoding the antibody could produce them. In other methods of producing herpesvirus antibodies, the identified antigen might be used for panning against a phage library. This procedure would isolate single chain phage-displayed antibodies in vitro.

A. Nucleic Acids

The present invention provides compositions comprising herpesvirus polynucleotides and methods of using these compositions to induce a protective immune response in vertebrate animals. In certain embodiments, an animal may be challenged with an herpesvirus infection.

In various embodiments of the invention, genes and polynucleotides encoding herpesvirus polypeptides, as well as fragments thereof, are provided. In other embodiments, a polynucleotide encoding an herpesvirus polypeptide or a polypeptide fragment may be expressed in prokaryotic or eukaryotic cells. The expressed polypeptides or polypeptide fragments may be purified for use as herpesvirus antigens in the vaccination of vertebrate animals or in generating antibodies immunoreactive with herpesvirus polypeptides or polypeptide fragments.

The present invention is not limited in scope to the genes of any particular virus of the Herpesvirus family. One of ordinary skill in the art could, using the nucleic acids described herein, readily identify related homologs in the Herpesvirus family. In addition, it should be clear that the present invention is not limited to the specific nucleic acids disclosed herein. As discussed below, a specific “herpesvirus” gene or polynucleotide fragment may contain a variety of different bases and yet still produce a corresponding polypeptide that is functionally indistinguishable, and in some cases structurally indistinguishable, from the polynucleotide sequences disclosed herein.

1. Nucleic Acids Encoding Herpesvirus Antigens

The present invention provides polynucleotides encoding antigenic herpesvirus polypeptides capable of inducing a protective immune response in vertebrate animals and for use as an antigen to generate anti-herpesvirus antibodies or antibodies reactive with other pathogens. In certain instances, it may be desirable to express herpesvirus polynucleotides encoding a particular antigenic herpesvirus polypeptide domain or sequence to be used as a vaccine, in generating anti-herpesvirus antibodies or in generating antibodies reactive with other pathogens. Nucleic acids according to the present invention may encode an entire HSV gene, or any other fragment of the HSV sequences set forth herein. The nucleic acid may be derived from PCR-amplified DNA of a particular organism. In other embodiments, however, the nucleic acid may comprise genomic DNA, complementary DNA (cDNA), or synthetically built DNA. A protein may be derived from the designated sequences for use in a vaccine or in methods for isolating antibodies.

The term “cDNA” is intended to refer to DNA prepared using messenger RNA (mRNA) as a template. The advantage of using a cDNA, as opposed to DNA amplified or synthesized from a genomic DNA template or a non-processed or partially processed RNA template is that a cDNA primarily contains coding sequences comprising the open reading frame (ORF) of the corresponding protein. There may be times when the full or partial genomic sequence is preferred, such as where the non-coding regions are required for optimal expression.

In still further embodiments, a herpesvirus polynucleotide from a given species may be represented by natural variants that have slightly different nucleic acid sequences but, nonetheless, encode the same polypeptide (see Table 1 below). In addition, it is contemplated that a given herpesvirus polypeptide from a species may be generated using alternate codons that result in a different nucleic acid sequence but encodes the same polypeptide.

As used in this application, the term “a nucleic acid encoding a herpesvirus polynucleotide” refers to a nucleic acid molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences are considered essentially the same as those set forth in a herpesvirus gene or polynucleotide that have at least about 50%, usually at least about 60%, more usually about 70%, most usually about 80%, preferably at least about 90% and most preferably about 95% of nucleotides that are identical to the nucleotides of a given herpesvirus gene or polynucleotide. Sequences that are essentially the same as those set forth in a herpesvirus gene or polynucleotide may also be functionally defined as sequences that are capable of hybridizing to a nucleic acid segment containing the complement of a herpesvirus polynucleotide under standard conditions. The term closely related sequences refers to sequences with either substantial sequence similarity or sequence that encode proteins that perform or invoke similar antigenic responses as described herein. The term closely related sequence is used herein to designate a sequence with a minimum of 50%, 60%, 70%, 80%, 90%, 95%, 96%, 97%, 98%, or 99% similarity with a polynucleotide or polypeptide with which it is being compared.

The DNA segments of the present invention include those encoding biologically functional equivalent herpesvirus proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes may be engineered through the application of site-directed mutagenesis techniques or may be introduced randomly and screened later for the desired function, as described below.

2. Non-bacterially Amplified Nucleic Acids

A nucleic acid or polynucleotide of the invention may be made by any technique known to one of ordinary skill in the art, such as for example, chemical synthesis, or enzymatic production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide or polynucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, and 5,602,244, each of which is incorporated herein by reference.

A non-limiting example of an enzymatically produced nucleic acid or polynucleotide includes one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference.

Another method for nucleic acid or polynucleotide amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention, see Wu et al., (1989), which is incorporated herein by reference in its entirety.

3. Oligonucleotides

Naturally, the present invention also encompasses oligonucleotides that are complementary, or essentially complementary to the sequences of an herpesvirus polynucleotide. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of an herpesvirus polynucleotide under relatively stringent conditions such as those described herein.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides or polynucleotides will typically find use, for example, as probes in Southern and Northern blots and as primers in amplification reactions or for vaccines.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Site-specific mutagenesis is a technique useful in the preparation of individual peptides, or biologically functional equivalent proteins or peptides, through specific mutagenesis of the underlying DNA. Typically, a primer of about 17 to 25 nucleotides in length is preferred, with about 5 to 10 residues on both sides of the junction of the sequence being altered (see Sambrook et al., 2001).

One method of using probes and primers of the present invention is in the search for genes related to the polynucleotides of HSV identified as encoding antigenic HSV polypeptides or, more particularly, homologs of HSV from other related viruses. Normally, the target DNA will be a genomic or cDNA library, although screening may involve analysis of RNA molecules. By varying the stringency of hybridization, and the region of the probe, different degrees of homology may be discovered (see Sambrook et al., 2001).

Another method of using oligonucleotides of the present invention is to design short RNA molecules for specific expression interference in vivo (siRNA).

B. Polypeptides and Antigens

For the purposes of the present invention a herpesvirus polypeptide, i.e., a polypeptide derived from a virus of the Herpesvirus family, may be a naturally-occurring polypeptide that has been identified by the methods described herein and extracted using protein extraction techniques well known to those of skill in the art. In particular embodiments, a herpesvirus antigen may be identified by ELI, RELI, or DELI and prepared in a pharmaceutically acceptable carrier for the vaccination of an animal.

In alternative embodiments, the herpesvirus polypeptide or antigen may be a synthetic peptide. In still other embodiments, the peptide may be a recombinant peptide produced through molecular engineering techniques. The present section describes the methods and compositions involved in producing a composition of herpesvirus polypeptides for use as antigens in the present invention.

1. Herpesvirus Polypeptides

Methods for screening and identifying herpesvirus genes that confer protection against herpesvirus infection are described herein. The herpesvirus polypeptide encoding genes or their corresponding cDNA may be inserted into an appropriate expression vector, LEE or CEE for the production of antigenic herpesvirus polypeptides. In addition, sequence variants of the polypeptide may be prepared. Polypeptide sequence variants may be minor sequence variants of the polypeptide that arise due to natural variation within the population or they may be homologs found in other viruses. They also may be sequences that do not occur naturally, but that are sufficiently similar that they function similarly and/or elicit an immune response that cross-reacts with natural forms of the polypeptide. Sequence variants can be prepared by standard methods of site-directed mutagenesis such as those described in Sambrook et al. 2001.

Another synthetic or recombinant variation of an antigenic herpesvirus polypeptide is a polyepitope moiety comprising repeats of epitope determinants found naturally in herpesvirus proteins. Such synthetic polyepitope proteins can be made up of several homomeric repeats of any one herpesvirus protein epitope; or may comprise of two or more heteromeric epitopes expressed on one or several herpesvirus protein epitopes.

Amino acid sequence variants of the polypeptide can be substitutional, insertional or deletion variants. Deletion variants lack one or more residues of the native protein which are not essential for function or immunogenic activity. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides that are homologs of the polypeptide. For example, an insertional variant could include portions of the amino acid sequence of the polypeptide from one species, together with portions of the homologous polypeptide from another species or subspecies. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide may be identified by an empirical approach in which portions of the gene encoding the polypeptide are expressed in a recombinant host, and the resulting proteins tested for their ability to elicit an immune response. For example, the polymerase chain reaction (PCR) can be used to prepare a range of cDNAs encoding peptides lacking successively longer fragments of the C-terminus of the protein. The immunogenic activity of each of these peptides then identifies those fragments or domains of the polypeptide that are essential for this activity. Further studies in which only a small number of amino acids are removed or added at each iteration then allows the location of other antigenic determinants of the polypeptide. Thus, use of the polymerase chain reaction, a technique for amplifying a specific segment of DNA via multiple cycles of denaturation-renaturation, using a thermostable DNA polymerase, deoxyribonucleotides and primer sequences is contemplated in the present invention (Mullis, 1990; Mullis et al., 1992).

Another embodiment for the preparation of the polypeptides according to the invention is the use of peptide mimetics. Mimetics are molecules that mimic elements of protein secondary structure. Because many proteins exert their biological activity via relatively small regions of their folded surfaces, their actions can be reproduced by much smaller designer (mimetic) molecules that retain the bioactive surfaces and have potentially improved pharmacokinetic/dynamic properties (Fairlie et al., 1998). Methods for mimicking individual elements of secondary structure (helices, turns, strands, sheets) and for assembling their combinations into tertiary structures (helix bundles, multiple loops, helix-loop-helix motifs) have been reviewed (Fairlie et al., 1998; Moore, 1994). Methods for predicting, preparing, modifying, and screening mimetic peptides are described in U.S. Pat. Nos. 5,933,819 and 5,869,451 (each specifically incorporated herein by reference). It is contemplated in the present invention, that peptide mimetics will be useful in screening modulators of an immune response.

Modifications and changes may be made in the sequence of a gene or polynucleotide and still obtain a molecule that encodes a protein or polypeptide with desirable characteristics. The following is a discussion based upon changing the amino acids of a protein or polypeptide to create an equivalent, or even an improved, second-generation molecule. The amino acid changes may be achieved by changing the codons of the DNA sequence, or by chemical peptide synthesis, according to the following examples.

For example, certain amino acids may be substituted for other amino acids in a polypeptide structure without appreciable loss of interactive binding capacity with structures such as, for example, antigen-binding regions of antibodies or binding sites on substrate molecules. Since it is the interactive capacity and nature of a polypeptide that defines the biological activity, certain amino acid substitutions can be made in a polypeptide sequence, and its underlying DNA coding sequence, and nevertheless obtain a polypeptide with like or improved properties. It is thus contemplated by the inventor that various changes may be made in the DNA sequences of the polynucleotides and genes of the invention without appreciable loss of their biological utility or activity. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It is known in the art that certain amino acids may be substituted by other amino acids having a similar hydropathic index or score and still result in a protein or polypeptide with similar biological activity. It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

It is also understood that an amino acid can be substituted for another having a similar hydrophilicity value and still obtain a biologically equivalent and immunologically equivalent protein.

Amino acid substitutions generally are based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine, as well as others.

2. Synthetic Polypeptides

Contemplated in the present invention are herpesvirus proteins and related peptides for use as antigens. In certain embodiments, the synthesis of an herpesvirus peptide fragment is considered. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference.

3. Polypeptide Purification

Herpesvirus polypeptides of the present invention are typically used as antigens for inducing a protective immune response in an animal and for the preparation of anti-herpesvirus antibodies. Thus, certain aspects of the present invention concern the purification, and in particular embodiments, the substantial purification, of a herpesvirus polypeptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies or by heat denaturation, which may be followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

To purify a desired protein, polypeptide, or peptide, which is a natural or recombinant composition comprising at least some specific proteins, polypeptides, or peptides will be subjected to fractionation to remove various other components from the composition. Various techniques suitable for use in protein purification will be well known to those of skill in the art. The most commonly used separative procedure for chemically synthesized peptides is HPLC chromatography. Other procedures for protein purification include affinity chromatography (e.g., immunoaffinity chromatography) and other methods known in the art. For exemplary methods and a more detailed discussion see Marshak et al. (1996) or Janson and Ryden (1998).

C. Polynucleotide Delivery

In certain embodiments of the invention, an expression construct comprising an herpesvirus polynucleotide or polynucleotide segment under the control of a heterologous promoter operable in eukaryotic cells is provided. For example, the delivery of an HSV-1 antigen-encoding expression constructs can be provided in this manner. The general approach in certain aspects of the present invention is to provide a cell with an expression construct encoding a specific protein, polypeptide or peptide fragment, thereby permitting the expression of the antigenic protein, polypeptide or peptide fragment in the cell. Following delivery of the expression construct, the protein, polypeptide or peptide fragment encoded by the expression construct is synthesized by the transcriptional and translational machinery of the cell and/or the vaccine vector. Various compositions and methods for polynucleotide delivery are known (see Sambrook et al., 2001; Liu and Huang, 2002; Ravid et al., 1998; and Balicki and Beutler, 2002, each of which is incorporated herein by reference).

Viral and non-viral delivery systems are two of the various delivery systems for the delivery of an expression construct encoding an antigenic protein, polypeptide, polypeptide fragment. Both types of delivery systems are well known in the art and are briefly described below. There also are two primary approaches utilized in the delivery of an expression construct for the purposes of genetic immunization; either indirect, ex vivo methods or direct, in vivo methods. Ex vivo gene transfer comprises vector modification of (host) cells in culture and the administration or transplantation of the vector modified cells to a subject. In vivo gene transfer comprises direct introduction of the vaccine vector into the subject to be immunized.

In various embodiments, a nucleic acid to be expressed may be in the context of a linear expression elements (“LEEs”) and/or circular expression elements (“CEEs”), which typically encompass a complete set of gene expression components (promoter, coding sequence, and terminator). These LEEs and CEEs can be directly introduced into and expressed in cells or an intact organism to yield expression levels comparable to those from a standard supercoiled, replicative plasmid (Sykes and Johnston, 1999). In some alternative methods and compositions of the invention, LEE or CEE allows any open-reading frame (ORF), for example, PCR™ amplified ORFs, to be non-covalently linked to an eukaryotic promoter and terminator. These quickly linked fragments can be directly injected into animals to produce local gene expression. It has also been demonstrated that the ORFs can be injected into mice to produce antibodies to the encoded foreign protein by simply attaching mammalian promoter and terminator sequences.

In certain embodiments of the invention, the nucleic acid encoding herpesvirus or similar polynucleotide may be stably integrated into the genome of a cell. In yet further embodiments, the nucleic acid may be stably or transiently maintained in a cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed. The following gene delivery methods provide the framework for choosing and developing the most appropriate gene delivery system for a preferred application.

1. Non-Viral Polynucleotide Delivery

In one embodiment of the invention, a polynucleotide expression construct may include recombinantly-produced DNA plasmids or in vitro-generated DNA. In various embodiments of the invention, an expression construct comprising, for example, a herpesvirus polynucleotide is administered to a subject via injection and/or particle bombardment (e.g., a gene gun). Polynucleotide expression constructs may be transferred into cells by accelerating DNA-coated microprojectiles to a high velocity, allowing the DNA-coated microprojectiles to pierce cell membranes and enter cells. In another preferred embodiment, polynucleotides are administered to a subject by needle injection. Injection of a polynucleotide expression construct may be given by intramuscular, intravenous, subcutaneous, intradermal, or intraperitoneal injection.

Particle Bombardment depends on the ability to accelerate DNA-coated microprojectiles to a high velocity allowing them to pierce cell membranes and enter cells without killing them (Klein et al., 1987). Several devices for accelerating small particles have been developed. The most commonly used forms rely on high-pressure helium gas (Sanford et al., 1991). The microprojectiles used have consisted of biologically inert substances such as tungsten or gold beads.

Transfer of an expression construct comprising herpesvirus or similar polynucleotides of the present invention also may be performed by any of the methods which physically or chemically permeabilize the cell membrane (e.g., calcium phosphate precipitation, DEAE-dextran, electroporation, direct microinjection, DNA-loaded liposomes and lipofectamine-DNA complexes, cell sonication, gene bombardment using high velocity microprojectiles and receptor-mediated transfection. In certain embodiments, the use of lipid formulations and/or nanocapsules is contemplated for the introduction of a herpesvirus polynucleotide, herpesvirus polypeptide, or an expression vector comprising a herpesvirus polynucleotide into host cells (see exemplary methods and compositions in Bangham et al., 1965; Gregoriadis, 1979; Dearner and Uster, 1983; Szoka and Papahadjopoulos 1978; Nicolau et al., 1987 and Watt et al., 1986; each of which is incorporated herein by reference). In another embodiment of the invention, the expression construct may simply consist of naked recombinant DNA, expression cassettes or plasmids.

2. Viral Vectors

In certain embodiments, it is contemplated that a herpesvirus gene or other polynucleotide that confers immune resistance to infection pursuant to the invention may be delivered by a viral vector. The capacity of certain viral vectors to efficiently infect or enter cells, to integrate into a host cell genome and stably express viral genes, have led to the development and application of a number of different viral vector systems (Robbins and Ghivizzani, 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488).

In particular embodiments, an adenoviral (U.S. Pat. Nos. 6,383,795; 6,328,958 and 6,287,571, each specifically incorporated herein by reference); retroviral (U.S. Pat. Nos. 5,955,331; 5,888,502; and 5,830,725, each specifically incorporated herein by reference); Herpes-Simplex Viral (U.S. Pat. Nos. 5,879,934 and 5,851,826, each specifically incorporated herein by reference in its entirety); Adeno-associated virus (AAV); poxvirus (e.g., vaccinia virus (Gnant et al., 1999)); alpha virus (e.g., sindbis virus; Semliki forest virus (Lundstrom, 1999)); reovirus (Coffey et al., 1998) and influenza A virus (Neumann et al., 1999); Chimeric poxviral/retroviral vectors (Holzer et al., 1999); adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982), expression vectors are contemplated for the delivery of expression constructs. “Viral expression vector” is meant to include those constructs containing virus sequences sufficient to (a) support packaging of the construct and (b) to ultimately express a tissue or cell-specific construct that has been cloned therein. Virus growth and manipulation is known to those skilled in the art.

D. Antibodies Reactive to Herpesvirus Antigens.

In another aspect, the present invention includes antibody compositions that are immunoreactive with a herpesvirus polypeptide of the present invention, or any portion thereof. In still other embodiments, an antigen of the invention may be used to produce antibodies and/or antibody compositions. Antibodies may be specifically or preferentially reactive to herpesvirus polypeptides. Antibodies reactive to herpesvirus include antibodies reactive to HSV, including those directed against an antigen having the sequences as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, fragments, variants, or mimetics thereof, or closely related sequences. The antigens of SEQ ID NO:2, SEQ ID NO:6, SEQ ID NO:10, SEQ ID NO:14, SEQ ID NO:22, SEQ ID NO:34, SEQ ID NO:42, SEQ ID NO:46, SEQ ID NO:50, SEQ ID NO:54, SEQ ID NO:58, SEQ ID NO:60, SEQ ID NO:62, SEQ ID NO:66, SEQ ID NO:70, SEQ ID NO:74, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, and SEQ ID NO:114 are representative of antigenic fragments of HSV polypeptides. Antigens represented in SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:40, SEQ ID NO:44, SEQ ID NO:48, SEQ ID NO:52, SEQ ID NO:56, SEQ ID NO:64, SEQ ID NO:68, SEQ ID NO:72, SEQ ID NO:78, SEQ ID NO:82, SEQ ID NO:86, SEQ ID NO:90, SEQ ID NO:94, SEQ ID NO:98, SEQ ID NO:102, SEQ ID NO:106, and SEQ ID NO:116 are exemplary of full length HSV polypeptides from which exemplary antigenic fragments have been identified. The antibodies may be polyclonal or monoclonal and produced by methods known in the art. The antibodies may also be monovalent or bivalent. An antibody may be split by a variety of biological or chemical means. Each half of the antibody can only bind one antigen and, therefore, is defined monovalent. Means for preparing and characterizing antibodies are well known in the art (see, e.g., Harlow and Lane, 1988, which is incorporated herein by reference).

Peptides corresponding to one or more antigenic determinants of a herpesvirus polypeptide of the present invention may be prepared in order to produce an antibody. Such peptides should generally be at least five or six amino acid residues in length, will preferably be about 10, 15, 20, 25 or about 30 amino acid residues in length, and may contain up to about 35 to 50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means. In other methods full or substantially full length polypeptides may be used to produce antibodies of the invention.

Once a peptide(s) is prepared that contains at least one or more antigenic determinants, the peptide(s) is then employed in the generation of antisera against the polypeptide. Minigenes or gene fusions encoding these determinants also can be constructed and inserted into expression vectors by standard methods, for example, using PCR cloning methodology. The use of peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

The antibodies used in the methods of the invention include derivatives that are modified, i.e, by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques, including, but not limited to, specific chemical cleavage, acetylation, formylation, and metabolic synthesis in the presence of tunicamycin. Additionally, the derivative may contain one or more non-classical amino acids.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a constant region derived, from a human immunoglobulin. Methods for producing chimeric antibodies are known in the art. See e.g., Morrison, 1985; O1 et al., 1986; Gillies et al. 1989; U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., U.S. Pat. No. 5,585,089 and Riechmann et al. (1988), which are incorporated herein by reference in their entireties. Antibodies can be humanized using a variety of techniques known in the art including, for example, CDR-grafting (EP 239,400; WO 91/09967; U.S. Pat. Nos. 5,225,539; 5,530,101 and 5,585,089), veneering or resurfacing (EP 592,106; EP 519,596; Padlan, 1991; Studnicka et al., 1994; Roguska et al., 1994), and chain shuffling (U.S. Pat. No. 5,565,332), all of which are hereby incorporated by reference in their entireties.

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,710,111; and WO 98/46645; WO 99/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar, 1995. For a detailed discussion of this technology for producing human antibodies and human monoclonal antibodies and protocols for producing such antibodies, see, e.g., PCT applications WO 98/24893; WO 92/01047; WO 96/34096; WO 96/33735; Europrean patent EP 0598877; U.S. Pat. Nos. 5,413,923; 5,625,126; 5,633,425; 5,569,825; 5,661,016; 5,545,806; 5,814,318; 5,885,793; 5,916,771; and 5,939,598; which are incorporated by reference herein in their entireties. In addition, companies such as Abgenix, Inc. (Freemont, Calif.). Kirin, Inc. (Japan), Medarex (N.J.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988).

The present invention encompasses single domain antibodies, including camelized single domain antibodies (See e.g., Muyldermans et al., 2001; Nuttall et al., 2000; Reichmann and Muyldermans, 1999; WO 94/04678; WO 94/25591; and U.S. Pat. No. 6,005,079; which are incorporated herein by reference in their entireties), In one embodiment, the present invention provides single domain antibodies comprising two VH domains with modifications such that single domain antibodies are formed.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater, than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof will increased in vivo half-lives can be generated by modifying (e.g., substituting, deleting or adding) amino acid residues identified as involved in the interaction between the Fc domain and the FcRn receptor. The antibodies of the invention may be engineered by methods described in Ward et al. to increase biological half-lives (see U.S. Pat. No. 6,277,375 B1). For example, antibodies of the invention maybe engineered in the Fc-hinge domain to have increased in vivo or serum half-lives.

Antibodies or fragments thereof with increased in vivo half-lives can be generated by attaching to the antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of the antibodies or antibody fragments or via episilon-amino groups present on lysine residues or other chemistry. Linear or branched polymer derivatization that results in minimal loss of biological activity will typically be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies of the invention may also be modified by the methods and coupling agents described by Davis et al. (U.S. Pat. No. 4,179,337) in order to provide compositions that can be injected into the mammalian circulatory system with substantially no immunogenic response.

In one aspect, the invention features multispecific, multivalent molecules, which minimally comprise an anti-Fc receptor portion, an anti-target portion and optionally an anti-enhancement factor (anti-EF) portion. In preferred embodiments, the anti-Fc receptor portion is an antibody fragment (e.g., Fab or (Fab′)₂ fragment), the anti-target portion is a ligand or antibody fragment and the anti-EF portion is an antibody directed against a surface protein involved in cytotoxic activity. In a particular embodiment, the recombinant anti-FcR antibodies, or fragments are “humanized” (e.g., have at least a portion of a complementarity determining region (CDR) derived from a non-human antibody (e.g., murine) with the remaining portion(s) being human in origin).

In various embodiments, the invention includes methods for generating multispecific molecules, e.g., a first specificity for an antigen and a second specificity for a Fc receptor. In one embodiment, both specificities are encoded in the same vector and are expressed and assembled in a host cell. In another embodiment, each specificity is generated recombinantly and the resulting proteins or peptides are conjugated to one another via sulfhydryl bonding of the C-terminus hinge regions of the heavy chain. In a particularly preferred embodiment, the hinge region is modified to contain only one sulfhydryl residue, prior to conjugation. For examples of these and other related methods and compositions see U.S. Pat. Nos. 6,410,690; 6,365,161; 6,303,755; 6,270,765; and 6,258,358 each of which are incorporated herein by reference. The present invention also encompasses the use of antibodies or antibody fragments comprising the amino acid sequence of any of the antibodies of the invention with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

The present invention also encompasses antibodies comprising a modified Fc region. Modifications that affect Fc-mediated effector function are well known in the art (U.S. Pat. No. 6,194,551, which is incorporated herein by reference in its entirety), for example, one or more amino acids alterations (e.g., substitutions) are introduced in the Fc region. The amino acids modified can be, for example, Proline 329, Proline 331, or Lysine 322. Proline 329, 331 and Lysine 322 are preferably replaced with alanine, however, substitution with any other amino acid is contemplated (PCT application WO 00/42072 and U.S. Pat. No. 6,194,551, which are incorporated herein by reference). In one particular embodiment, the modification of the Fc region comprises one or more mutations in the Fc region. In another particular embodiment, the modification in the Fc region has altered antibody-mediated effector function. In another embodiment of the invention, the modification in the Fc region has altered binding to other Fc receptors (e.g., Fc activation receptors). In yet another particular embodiment, the antibodies of the invention comprising a modified Fc region mediate ADCC more effectively. In another embodiment, the modification in the Fc region alters C1q binding activity. In yet a further embodiment, the modification in the Fc region alters complement dependant cytotoxicity.

The invention also comprises antibodies with altered carbohydrate modifications (e.g., glycosylation, fucosylation, etc.), wherein such modification enhances antibody-mediated effector function. Carbohydrate modifications that lead to altered antibody mediated effector function are well known in the art (for example see Shields et al., 2001; Davies et al., 2001).

1. Antibody Conjugates

The present invention encompasses antibodies recombinantly fused or chemically conjugated (including both covalently and non-covalent conjugations) to heterologous polypeptides (i.e., an unrelated polypeptide; or portion thereof, preferably at least 10, at least 20, at least 30, at least 40, at least 50, at least 60, at least 70, at least 80, at least 90, or at least 100 amino acids of the polypeptide) to generate fusion proteins. The fusion does not necessarily need to be direct, but may occur through linker sequences. Antibodies may be used for example to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in, the art, see e.g., PCT application WO 93/21232; European patent EP 439,095; Naramura et al., 1994; U.S. Pat. No. 5,474,981; Gillies et al., 1992; and Fell et al., 1991, which are incorporated herein by reference in their entireties.

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin (i.e., PE-40), or diphtheria toxin, ricin, gelonon, and pokeweed antiviral protein or other toxin, a protein such as tumor necrosis factor, interferons including, but not limited to, alpha-interferon (IFN-α), beta-interferon (IFN-β), nerve growth factor (NGF), platelet derived growth factor (PDGF), tissue plasminogen activator (TPA), an apoptotic agent (e.g., TNF-α, TNF-β, AIM I (PCT application WO 97/33899), AIM II (PCT application WO 97/34911), Fas Ligand (Takahashi et al., 1994), and VEGI (PCT application WO 99/23105), a thrombotic agent or an anti-angiogenic agent (e.g., angiostain or endostatin), or a biological response modifier such as, for example, lymphokine (e.g. interleukm-1 (“IL-1”), interleukin-2 (“IL-2”), interleukm-6 (“IL-6”) granulocyte macrophage colony stimulating factor (“GM-CSF”), and granulocyte colony stimulating factor (“G-CSF”), macrophage colony stimulating factor, (“M-CSF”), or a growth factor (e.g., growth hormone (“GH”); proteases, or ribonucleases.

Antibodies can be fused to marker sequences, such as a peptide to facilitate purification. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., 1989, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984) and the “flag” tag (Knappik et al., 1994).

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See for example U.S. Pat. Nos. 5,336,603; 5,622,929; 5,359,046; 5,349,053; 3,447,851; and 5,112,946; Eurppean Patents EP 307,434 and EP 367,166; PCT applications WO 96/04388 and WO 91/06570; Ashkenazi et al., 1991, PNAS 88: 10535-10539; Zheng et al., 1995; and Vil et al., 1992; each of which are incorporated by reference in there entireties).

Additional fusion proteins may be generated through the techniques of gene-shuffling, motif-shuffling, exon-shuffling; and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates), see, generally, U.S. Pat. Nos. 5,605,793; 5,811,238; 5,830,721; 5,834,252; and 5,837,458; and Patten et al., 1997; Harayama, 1998; Hansson et al., 1999; Lorenzo and Blasco, 1998; each of which are hereby incorporated by reference in its entirety. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to FcγRIIB may be recombined with one or more components, motifs, sections, parts, domains, fragments, etc. of one or more heterologous molecules.

The present invention also encompasses antibodies conjugated to a diagnostic or therapeutic agent or any other molecule for which serum half-life is desired to be increased. The antibodies can be used diagnostically to, for example, monitor the development or progression of a disease, disorder or infection as part of a clinical testing procedure, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and non-radioactive paramagnetic metal ions. The detectable substance may be coupled or conjugated either directly to the antibody or indirectly, through an intermediate (such as, for example, a linker known in the art) using techniques known in the art, see, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzyme, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to umbelliferone, fluorescein, fluorescein isothiocyanate, rhodamine, dichlorotriazinylamine, fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³B), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹n), iodine (¹³¹I, ¹²⁵I, ¹²³I, ¹²¹), lansthanium (¹⁴⁰La), lutetitium (¹⁷⁷Lu), manganese (⁵⁴Mn), molybdenum (⁹⁹Mo), palladium (¹⁰³Pd), phosphorous (³²p), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), titanium (⁴⁴Ti), tin (¹¹³Sn, ¹¹⁷Sn), tritium (³H), xenon (¹³⁶Xe), ytterbium (¹⁷⁹Yb, ¹⁷⁵Yb), yttrium (⁹⁰Y), zinc (⁶⁵Zn); positron emitting metals using various positron emission tomographies, and non-radioactive paramagnetic metal ions.

An antibody may be conjugated to a therapeutic moiety such as a. cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anrhracindione, mitoxantrone. mithramycin, actinciomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine; cytarabine, 5-fluorouracil decarbazine), alkylating agents (e.g., mechlorethamine, thioepa Chlorambucil, melphalan, carmustine (BSNU) and lomustine (CCNU), cyclophosphamide, busulfan, dibromomannitol, streptozotocin, mitomycin C, and cisdichlorodiamine platinum (II) (DDP) cisplatin.), anthracyclines (e.g., daunorubicin (formerly daunomycin) and doxorubicin). antibiotics (e.g., dactinomycin (fomerly actinomycin), bleomycin, mithramycin, and anthramycin (AMC)), and anti-mitotic agents (e.g., vincristine and vinblastine).

Moreover, an antibody can be conjugated to therapeutic moieties such as a radioactive materials or macrocyclic chelators useful for conjugating radiometal ions (see above for examples radioactive materials). In certain embodiments, macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998; Peterson et al., 1999; and Zimmerman et al., 1999, each incorporated by reference in their entireties.

Techniques for conjugating such therapeutic moieties to antibodies are well known; see, example Arnon et al., 1985; Hellstrom et al., 1987; Thorpe, 1985; Thorpe et al., 1982.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal (U.S. Pat. No. 4,676,980, which is incorporated herein by reference in its entirety.

Antibodies may also be attached to solid supports that are particularly useful for immunoassays or purification of the target antigen. Such solid supports include, but are not limited to, glass, cellulose, polyacrylamide, nylon, polystyrene, polyvinyl chloride or polypropylene.

2. Anti-Herpesvirus Antibody Generation

The present invention provides monoclonal antibody compositions that are immunoreactive with a herpesvirus polypeptide. As detailed above, in addition to antibodies generated against a full-length herpesvirus polypeptide, antibodies also may be generated in response to smaller constructs comprising epitope core regions, including wild-type and mutant epitopes. In other embodiments of the invention, the use of anti-herpesvirus single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

However, “humanized” herpesvirus antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-HSV humanized or chimeric antibody can be genetically engineered to comprise an HSV antigen binding site of a given of molecular weight and biological lifetime, as long as the antibody retains its HSV antigen binding site. Humanized antibodies may be prepared by using following the teachings of U.S. Pat. No. 5,889,157

The term “antibody” is used to refer to any antibody-like molecule that has an antigen binding region, and includes antibody fragments such as Fab′, Fab, F(ab′)₂, single domain antibodies (DABs), Fv, scFv (single chain Fv), chimeras and the like. Methods and techniques of producing the above antibody-based constructs and fragments are well known in the art (U.S. Pat. Nos. 5,889,157; 5,821,333; and 5,888,773, each specifically incorporated herein by reference). The methods and techniques for preparing and characterizing antibodies are well known in the art (see, e.g., Antibodies: A Laboratory Manual, Cold Spring Harbor Laboratory, 1988; incorporated herein by reference).

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions. In addition to adjuvants, it may be desirable to coadminister biologic response modifiers (BRM), which have been shown to upregulate T cell immunity or downregulate suppressor cell activity.

3. Detecting Herpesvirus

The invention also relates to methods of assaying for the presence of herpesvirus infection, in particular HSV-1 or HSV-2 infection, in a patient, subject, vertebrate animal, and/or human comprising: (a) obtaining an antibody, as described above, directed against a herpesvirus antigen of the invention; (b) obtaining a sample from a subject, patient, and/or animal; (c) admixing the antibody with the sample; and (d) assaying the sample for antigen-antibody binding, wherein the antigen-antibody binding indicates herpesvirus infection in the animal. In some cases, the antibody directed against the antigen is further defined as a polyclonal antibody. In other embodiments, an antibody directed against the antigen is further defined as a monoclonal antibody. In some embodiments, an antibody is reactive against an antigen having a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, fragments, variants, or mimetics thereof, or closely related sequences. The assaying of the sample for antigen-antibody binding may be by precipitation reaction, radioimmunoassay, ELISA, Western blot, immunofluorescence, or any other method known to those of skill in the art.

In other embodiments, the invention also relates to methods of assaying for the presence of herpesvirus infection or antibodies reactive to herpesvirus, in particular HSV-1 or HSV-2 infection, in a patient, subject, vertebrate animal, and/or human comprising: (a) obtaining a peptide, as described above; (b) obtaining a sample from a subject, patient, and/or animal; (c) admixing the peptide with the sample; and (d) assaying the sample for antigen-antibody binding, wherein the antigen-antibody binding indicates exposure of the animal to herpesvirus. The peptide may have a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, fragments, variants, or mimetics thereof, or closely related sequences. The assaying of the sample for antigen-antibody binding may be by precipitation reaction, radioimmunoassay, ELISA, Western blot, immunofluorescence, or any other method known to those of skill in the art.

The invention further relates to methods of assaying for the presence of an HSV infection in an animal comprising: (a) obtaining an oligonucleotide probe comprising a sequence comprised within one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 and/or SEQ ID NO:115, a complement, a fragment, or a closely related sequences thereof; and (b) employing the probe in a PCR or other detection protocol.

E. Other Binding or Affinity Agents

Various embodiments of the invention may include the use of alternative binding or affinity agents that preferentially bind nucleic acids and/or polypeptides, including fragments, portions, subdivisions and the like, of nucleic acids or polypeptides, including variants thereof, of the present invention. A binding agent may include nucleic acids, amino acids, synthetic polymers, carbohydrates, lipids, and combinations thereof as long as the compound, molecule, or complex preferentially binds or has a measurable affinity, as determined by methods known in the art, for a nucleic acid or polypeptide of the present invention. The binding affinity of an agent can, for example, be determined by the Scatchard analysis of Munson and Pollard, 1980. Other binding agents may include, but are not limited to nucleic acid aptamers; anticalins or other lipocalin derivatives (for examples see U.S. Pat. Nos. 5,506,121 and 6,103,493; PCT applications WO 99/16873 and WO 00/75308 and the like); synthetic or recombinant antibody derivatives (for examples see U.S. Pat. No. 6,136,313. Exemplary methods and compositions may be found in U.S. Pat. Nos. 5,506,121 and 6,103,493 and PCT applications WO 99/16873 and WO 00/75308 and the like, each of which is incorporated herein by reference. Any binding or affinity agents derived using the compositions of the present invention may be used in therapeutic, prophylactic, vaccination and/or diagnostic methods.

### V. Therapeutic Compositions and Methods

It is further contemplated that the compositions and methods of the invention may be used as a therapeutic composition for viral infections. The therapeutics may be used to treat and/or diagnose viral infection. In certain embodiments, the nucleic acid and/or polypeptides of the invention may be used as a therapeutic agent. In various embodiments of the invention antibodies, binding agents, or affinity agents that recognize and/bind the nucleic acids or polypeptides of the invention may be used as therapeutic agents. These therapeutic compositions may act through mechanisms that include, but are not limited to the induction or stimulation of an active immune response by an organism or subject. Such therapeutic methods include passive immunization, prime-boost immunization, and other methods of using antigens, vaccines, and/or antibodies or other binding agents to protect, prevent, and/or treat infection by a pathogen.

Antibodies or binding agents of the invention may be conjugated to a therapeutic agent. Therapeutic agents may include, but are not limited to apoptosis-inducing agents, toxins, anti-viral agents, pro-drug converting enzymes and any other therapeutic agent that may aid in the treatment of a viral infection(s). Compositions of the present invention may be used in the targeting of a therapeutic agent to a focus of infection, the method of which may include injecting a patient infected with a pathogen with an effective amount of an antibody-therapeutic agent conjugate. The conjugate may include an immunoreactive composite of one or more chemically-linked antibodies or antibody fragments which specifically binds to a one or more epitopes of one or more pathogens or of an antigen induced by the pathogen or presented by a cell as a result of the fragmentation or destruction of the pathogen at the focus of infection. The antibody conjugate may have a chemically bound therapeutic agent for treating said infection, thus localizing or targeting a therapeutic to the location of a pathogen.

Reviews of antimicrobial chemotherapy can be found in the chapter by Slack, 1987 and in Section XII, Goodman and Gilman's The Pharmacological Basis of Therapeutics, 1980).

As indicated in these texts, some antimicrobial agents are selective in their toxicity, since they kill or inhibit the microorganism at concentrations that are tolerated by the host (i.e., the drug acts on microbial structures or biosynthetic pathways that differ from those of the host's cells). Other agents are only capable of temporarily inhibiting the growth of the microbe, which may resume growth when the inhibitor is removed. Often, the ability to kill or inhibit a microbe or parasite is a function of the agent's concentration in the body and its fluids.

Whereas these principles and the available antimicrobial drugs have been successful for the treatment of many infections, particularly bacterial infections, other infections have been resistant or relatively unresponsive to systemic chemotherapy, e.g., viral infections and certain fungal, protozoan and parasitic infections.

As used herein, “microbe” denotes virus, bacteria, rickettsia, mycoplasma, protozoa and fungi, while “pathogen” denotes both microbes and infectious multicellular invertebrates, e.g., helminths, spirochetes and the like.

Virus can infect host cells and “hide” from circulating systemic drugs. Even when viral proliferation is active and the virus is released from host cells, systemic agents can be insufficiently potent at levels which are tolerated by the patient. Thus, the compositions of the invention may be used in targeting therapeutics to the location that will typically be more effective in treating an infection by a pathogen.

A. Prime-Boost Vaccination Methods

When one or more compositions of the invention are administered in conjunction with or without adjuvants and/or other excipients, the antigen may be administered before, after, and/or simultaneously with the other antigenic compositions. For instance, the combination of antigens or vaccine compositions may be administered as a priming dose of antigen or vaccine composition. One or more antigen or vaccine composition may then be administered with a boost dose, including the antigen or vaccine composition used as the priming dose. Alternatively, the combination of two or more antigens or vaccine compositions may be administered with a boost dose of antigen. One or more antigen or vaccine composition may then be administered with the prime dose. A “prime dose” is the first dose of antigen administered to a subject. In the case of a subject that has an infection the prime dose may be the initial exposure of the subject to the pathogen and a combination of antigens or vaccine compositions may administered to the subject in a boost dose. A “boost dose” is a second, third, fourth, fifth, sixth, or more dose of the same or different antigen or vaccine composition administered to a subject that has already been exposed to an antigen. In some cases the prime dose may be administered with a combination of antigens or vaccine compositions such that a boost dose is not required to protect a subject at risk of infection from being infected. An antigen may be administered with one or more adjuvants or other excipients individually or in any combination. Adjuvants may be administered prior to, simultaneously with or after administration of one or more antigen(s) or vaccine compositions. It is contemplated that repeated administrations of antigen(s) as well as one or more of the components of a vaccine composition may be given alone or in combination for one or more of the administrations. Antigens need not be from a single pathogen and may be derived from one or more pathogens. The order and composition of a vaccine composition may be readily determined by using known methods in combination with the teachings described herein. Examples of the prime-boost method of vaccination can be found in U.S. Pat. No. 6,210,663, incorporated herein by reference.

In various embodiment, the time between administration of the priming dose and the boost dose maybe 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more days, weeks, months, or years. The vaccine compositions include, but are not limited to any of the polynucleotide, polypeptide, and binding agent compositions described herein or combination of any 1, 2, 3, 4, 5, 6, 7, 8, 9, 10, 11, 12, 13, 14, 15, 16, 17, 18, 19, 20, or more of each individual composition.

B. Passive Immunization

Methods of passively immunizing an animal or human subject against a preselected ligand or pathogen by administering to the animal or human subject a composition comprising one or more antibodies or affinity agents to an antigen(s) of the present invention are contemplated.

Immunoglobulin molecules and other affinity or binding agents are capable of binding a preselected antigen and can be efficiently and economically produced synthetically and in plant or animal cells as well as in a variety of animals including, but not limited to horse, pig, rabbit, goat, donkey, mouse, rat, human and other organisms capable of producing natural or recombinant molecules. In certain cases, immunoglobulin molecules may or may not contain sialic acid yet do contain core glycosylated portions and N-acetylglucosamine containing outer branches. In various embodiments, an immunoglobulin molecule either is an IgA, IgM, secretory IgM or secretory IgA.

Secretory immunoglobulins, such as secretory IgM and secretory IgA may be resistant to proteolysis and denaturation. Contemplated environments for the administration or use of such molecules include acidic environments, protease containing environments, high temperature environments, and other harsh environments. For example, the gastrointestinal tract of an animal is a harsh environment where both proteases and acid are present, see, Kobayishi et al., 1973. Passive immunization of an animal or human subject may be produced by contacting or administering an antibody or binding agent that recognizes an antigen of the present invention by intravascular, intramuscular, oral, intraperitoneal, mucosal, or other methods of administration. Mucosal methods of administration may include administration by the lungs, the digestive tract, the nasopharyngeal cavity, the urogenital system, and the like.

In various embodiments the antibody or binding agent, such as an immunoglobulin molecule is specific for a preselected antigen. Typically, this antigen is present on a pathogen that causes a disease. One or more antibody or binding agent may be capable of binding to a pathogen(s) and preventing or treating a disease state.

In certain embodiments, the composition comprising one or more antibody or binding agent is a therapeutic or pharmaceutically acceptable composition. The preparation of therapeutic or pharmaceutically acceptable compositions which contain polypeptides, proteins, or other molecules as active ingredients is well understood in the art and are briefly described herein.

In certain embodiments, a composition containing one or more antibody or binding agent(s) comprises a molecule that binds specifically or preferentially with a pathogen antigen. Preferentially is used herein to denote that a molecule may bind other antigens or molecules but with a much lower affinity as compared to the affinity for a preferred antigen. Pathogens may be any organism that causes a disease in another organism.

Antibodies or binding agents specific or preferential for a pathogen may be produced using standard synthetic, recombinant, or antibody production techniques, see, Antibodies: A Laboratory Manual, Harlow et al., eds., Cold Spring Harbor, N.Y. (1988) and alternative affinity or binding agents described herein.

C. Therapeutic Vaccination

A promising use of vaccination is the use of therapeutic vaccination to treat or cure established diseases or infections. Methods of therapeutically immunizing an animal or human subject against a preselected ligand or pathogen by contacting or administering to the animal or human subject a composition comprising one or more antigen(s) of the present invention are contemplated. Therapeutic vaccinations may provided relief of complications of, for example, lesions or precursor lesions resulting from herpesvirus infection, and thus represent an alternative to prophylactic intervention. Vaccinations of this type may comprise various polypeptides or polynucleotides as described herein, which are expressed in persistently infected cells. It is assumed that following administration of a vaccination of this type, cytotoxic T-cells might be activated against persistently infected cells in the lesions associated with infection or disease.

Vaccine candidates of the present invention may be prepared or combined for delivery into an infected subject for the treatment of the infection. It is anticipated that the immune responses raised against these antigens might be capable of eliminating the resident pathogen or preventing or ameliorating disease symptoms associated with herpes reactivation.

### VI. Microbial Genomics

Automated-DNA sequencing has revolutionized the investigation of pathogenic microbes by making the entirety of the information contained within their genomes available for analysis. The availability of genomic and/or proteomic information may be used in context of the invention described herein. In certain embodiments, genomic or proteomic information may be used for the analysis of a pathogenic organism's genome and for identification of polynucleotides or polypeptides encoded by polynucleotides for the purpose of vaccination, vaccine preparation, antibody preparation, and the like. Genomic techniques, methods, and composition have been designed to extract knowledge from sequence data (protein and DNA), microarray data, and other genomic based data. One application of whole-genome-sequence information is investigation of the pathogenic role of microbial genes and their candidacy as a vaccine. The availability of a large number of sequenced microbial genomes allows the systematic study and analysis of microbial genes.

The genomic sequences of a large number of medically and agriculturally important organisms are or will be known. Genomic technologies are particularly attractive for addressing complex questions that are becoming evident with the increase in sequence information. Many conventional genetic and biochemical approaches have their limitations, especially in regard to some pathogenic organisms.

The rapidly developing fields of genomics, proteomics and bioinformatics rely on various techniques including, but not limited to, mass spectrometry, high performance chromatography and electrophoresis, protein sequencing and other genomic or proteomic technologies (see Cunningham, 2000 for a general review). Also, development, advancement and integration of proteomics technologies and other areas related to functional genomics, including primary structure determination, chemical modification of proteins, protein-protein crosslinking mass spectrometry, protein purification and characterization and process engineering.

Genomic applications include, but are not limited to enriched haplotyping, expression analysis, bio-defense and microbial analysis. Using direct, linear readings of long, unbroken segments of DNA, it has the potential to capture comprehensive genetic data, offering researchers a technology to decode genomes, identify genetic variations, and enable pharmacogenomics, drug discovery, population genetics, and agbiotech applications.

A. Genomic Technologies

Various genomic methods and techniques may be utilized during the analyses of a pathogen. For example gene synthesis (for exemplary methods see U.S. Pat. Nos. 6,472,184 and 6,110,668); genotyping (for exemplary methods see U.S. Pat. Nos. 5,846,704 and 6,449, 562); library construction (for exemplary methods see U.S. Pat. No. 6,468,765 and Sambrook et al., 2001); oligo synthesis, including modified oligo and RNA oligo synthesis (Ausubel, et al., 1993 or Integrated DNA Technologies, Coralville, Iowa), as well as sequencing and synthesis services that are commercially available (e.g., Qiagen Genomics, Bothell, Wash.; or Cleveland Genomics, Cleveland, Ohio)

B. Animal Models

Various assay used to provide information regarding the function of a gene or protein utilize transgenic organisms. Animal models include transgenic animals, transgenic mice, transgenic murine cell lines, transgenic rat cell lines, or transgenic rats.

C. Array Technology

Various arrray technologies also are available for genomic and proteomic analyses (Bowtell et al., 2003). Arrays include, but are not limited to Antibody Arrays (BD Biosciences Clontech, Palo Alto, Calif.); cDNA Arrays (Incyte Genomics, St. Louis, Mo.), Microbial Arrays (Sigma-Genosys, The Woodlands, Tex.), Oligo Arrays (QIAGEN Operon, Alameda, Calif.); Protein—DNA Interaction Arrays (BD Biosciences Clontech, Palo Alto, Calif.); Protein Arrays (Ciphergen Biosystems, Inc., Fremont, Calif.); and other types of arrays available from various vendors.

D. Robotics

Various robotic or automated machines are typically used in conjunction with high-throughput methods associated with genomics and proteomics. Exemplary robots or machines include Automated Colony Pickers/Arrayers (Biorad, Hercules Calif.; and Genetix, Beaverton Oreg.); Automated Dispensers, Microplate Handlers, Microplate Washers (Beckman Coulter, Fullerton Calif.; Bio-Tek Instruments, Winooski Vt.; and PerkinElmer Life Sciences Inc., Boston Mass.); Automated Nucleic Acid/Protein Analysis (Beckman Coulter, Fullerton Calif.), Automated Nucleic Acid Purification (QIAGEN, Valencia Calif.); Automated Protein Expression Instruments (Roche Applied Science, Indianapolis Ind.); and High Throughput Fluorescence Detection (Cellomics, Inc., Pittsburgh Pa.).

### VI. Pharmaceutical Compositions

Compositions of the present invention comprise an effective amount of a Herpesvirus polynucleotide or variant thereof; an antigenic protein, polypeptide, peptide, or peptide mimetic; anti-herpesvirus antibodies; and the like, which may be dissolved and/or dispersed in a pharmaceutically acceptable carrier and/or aqueous medium. Aqueous compositions of genetic immunization vectors, vaccines and such expressing any of the foregoing are also contemplated.

A. Pharmaceutical Preparations of Peptides, Nucleic Acids, and Other Active Compounds.

The herpesvirus polypeptides of the invention and the nucleic acids encoding them may be delivered by any method known to those of skill in the art (see for example, “Remington's Pharmaceutical Sciences” 15th Edition).

Solutions comprising the compounds of the invention may be prepared in water suitably mixed with a surfactant, such as hydroxypropylcellulose. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. The form should usually be sterile and must be fluid to the extent that easy syringability exists. It must be stable under the conditions of manufacture and storage and must be preserved against the contaminating action of microorganisms, such as bacteria and fungi.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

For human administration, preparations should meet sterility, pyrogenicity, general safety and purity standards as required by FDA, Center for Biologics Evaluaiton and Research and the Center for Drug Evaluation and Research.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

B. Routes of Delivery/Administration

Pharmaceutical compositions may be conventionally administered parenterally, by injection, for example, either subcutaneously, intradermally, or intramuscularly. However, any method for administration of a composition is applicable. These include gene gun inoculation of the DNA encoding the peptide(s), oral application on a solid physiologically acceptable base or in a physiologically acceptable dispersion, transdermal patch application, parenteral delivery, injection, or the like. The polynucleotides and polypeptides of the invention will typically be formulated for parenteral administration, such as injection via the intravenous, intramuscular, sub-cutaneous, intralesional, epidermal, transcutaneous, intraperitoneal routes. Additionally, compositions may be formulated for oral, intravaginal or inhaled delivery.

Injection of a nucleic acid encoding a herpesvirus polypeptide may be delivered by syringe or any other method used for injection of a solution, as long as the nucleic acid encoding the herpesvirus polypeptide, can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

C. Adjuvants

Immunogenicity can be significantly improved if the vectors or antigens are co-administered with adjuvants. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Adjuvants can stimulate or signal activation of cells or factors of the immune system. Exemplary adjuvants may be found in U.S. Pat. No. 6,406,705, incorporated herein by reference.

As used herein, the term “adjuvant” refers to an immunological adjuvant. By this is meant a compound that is able to enhance the immune system's response to an immunogenic substance or antigen. The term “immunogenic” refers to a substance or active ingredient which when administered to a subject, either alone or with an adjuvant, induces an immune response in the subject. The term “immune response” includes specific humoral, i.e. antibody, as well as cellular immune responses, the antibodies being serologic as well as secretory and pertaining to the subclasses IgM, IgD, IgG, IgA and IgE as well as all isotypes, allotypes, and subclasses thereof. The term is further intended to include other serum or tissue components. The cellular response includes Type-1 and Type-2 T-helper lymphocytes, cytotoxic T-cells as well as natural killer (NK) cells.

Furthermore, several other factors relating to adjuvanicity are believed to promote the immunogenicity of antigens. These include (1) rendering antigens particulate, e.g. aluminum salts, (2) polymers or polymerization of antigens, (3) slow antigen release, e.g. emulsions or micro-encapsulation, (4) bacteria and bacterial products, e.g. CFA, (5) other chemical adjuvants, e.g. poly-I:C, dextran sulphate and inulin, (6) cytokines, and (7) antigen targeting to APC.

General categories of adjuvants that may be used in conjunction with the invention includes, but is not limited to peptides, nucleic acids, cytokines, microbes (bacteria, fungi, parasites), glycoproteins, glycolipids, lipopolysaccharides, emulsions, and the like.

A combination of adjuvants may be administered simultaneously or sequentially. When adjuvants are administered simultaneously they can be administered in the same or separate formulations, and in the latter case at the same or separate sites, but are administered at the same time. The adjuvants are administered sequentially, when the administration of at least two adjuvants is temporally separated. The separation in time between the administrations of the two adjuvants may be a matter of minutes or it may be longer. The separation in time is less than 14 days, and more preferably less than 7 days, and most preferably less than 1 day. The separation in time may also be with one adjuvant at prime and one at boost, or one at prime and the combination at boost, or the combination at prime and one at boost.

In some embodiments, the adjuvant is Adjumer™, Adju-Phos, Algal Glucan, Algammulin, Alhydrogel, Antigen Formulation, Avridine®, BAY R1005, Calcitriol, Calcium Phosphate Gel, Cholera holotoxin (CT), Cholera toxin B subunit (CTB), Cholera toxin A1-subunit-Protein A D-fragment fusion protein, CRL1005, Cytokine-containing Liposome, Dimethyldioctadecylammonium bromide, Dehydroepiandrosterone; Dimyristoyl phosphatidylcholine; 1,2-dimyristoyl-sn-3-phosphatidylcholine, Dimyristoyl phosphatidylglycerol, Deoxycholic Acid Sodium Salt; Freund's Complete Adjuvant, Freund's Incomplete Adjuvant, Gamma Inulin, Gerbu Adjuvant, GM-CSF, N-acetylglucosaminyl-(β1-4)-N-acetylmuramyl-L-alanyl-D-isoglutamine, Imiquimod, ImmTher™, Interferon-1α, Interleukin-1β, Interleukin-2, Interleukin-7, Interleukin-12, ISCOM™, Iscoprep 7.0.3.™, Liposome, Loxoribine, LT-OA or LT Oral Adjuvant, MF59, MONTANIDE ISA 51, MONTANIDE ISA 720, MPL™, MTP-PE, MTP-PE Liposome, Murametide, Murapalmitine, D-Murapalmitine, NAGO, Non-Ionic Surfactant Vesicle, Pleuran, lactic acid polymer, glycolic acid polymer, Pluronic L121, Polymethyl methacrylate, PODDS™, Poly rA:Poly rU, Polysorbate 80, Protein Cochleate, QS-21, Quil-A, Rehydragel HPA, Rehydragel LV, S-28463, SAF-1, Sclavo peptide, Sendai Proteoliposome, Sendai-containing Lipid Matrix, Span 85, Specol, Squalane, Squalene, Stearyl Tyrosine, Theramide™, Threonyl-MDP, Ty Particle, or Walter Reed Liposome.

D. Dosage and Schedules of Administration

The dosage of the polynucleotides and/or polypeptides and dosage schedule may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

Administration is in any manner compatible with the dosage formulation, and in such amount as will be therapeutically effective and/or immunogenic. The quantity to be administered depends on the subject to be treated, including the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner.

In certain embodiments, pharmaceutical compositions may comprise, for example, at least about 0.1% of an active compound. One of the various active compounds being a herpesvirus polynucleotide or polypeptide. In other embodiments, an active compound may comprise between about 2% to about 75% of the weight of the unit, or between about 25% to about 60%, for example, and any range derivable therein. However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations of a vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters after the initial series of immunizations at intervals of 1-5 years, usually three years, will be desirable to maintain protective levels of the antibodies.

A course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with a nucleic acid can be performed, following immunization.

### VII. Kits

The invention also relates to kits for assaying an HSV infection comprising, in a suitable container: (a) a pharmaceutically acceptable carrier; and (b) an antibody, or other suitable binding agent, directed against an HSV antigen.

Therapeutic kits of the present invention are kits comprising a herpesvirus (e.g., HSV-1 or HSV-2) polynucleotide or polypeptide or an antibody to the polypeptide. Such kits will generally contain, in a suitable container, a pharmaceutically acceptable formulation of an herpesvirus polynucleotide or polypeptide, or an antibody to the polypeptide, or vector expressing any of the foregoing in a pharmaceutically acceptable formulation. The kit may have a single container, and/or it may have a distinct container for each compound.

When the components of the kit are provided in one and/or more liquid solutions, the liquid solution is an aqueous solution, with a sterile aqueous solution being particularly preferred. The herpesvirus polynucleotide or polypeptide, or antibody compositions may also be formulated into a syringeable composition. In which case, the container may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container.

The container will generally include at least one vial, test tube, flask, bottle, syringe and/or other container, into which the herpesvirus polynucleotide or polypeptide, or antibody formulation are placed, preferably, suitably allocated. The kits may also comprise a second container for containing a sterile, pharmaceutically acceptable buffer and/or other diluent.

The kits of the present invention will also typically include a means for containing the vials in close confinement for commercial sale, such as, injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate herpesvirus polynucleotide or polypeptide, or an antibody to the polypeptide within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A RELI Screen: Construction of Libraries

### Expressing Herpes Simples Virus 1 (HSV-1) DNA

Genomic DNA from the MacIntryre strain of HSV-1 was purified from cultured green monkey kidney cells (VERO-E6). The viral DNA was physically sheared by nebulization, purified and size-selected by electrophoresis through a 1.5% agarose TRIS-borate gel. Fragments from 500 to 2000 base pairs (bp) were excised and electroeluted. The library production protocol was similar to that previously described to generate HIV random expression libraries (Sykes and Johnston, 1999, incorporated herein by reference). However instead of attaching adaptors to the sheared fragments to generate BglII restriction site overhangs, the fragments were enzymatically mended (Klenow and T4 polymerase) to generate blunt-ends. The mended fragments were ligated into two mammailian expression plasmids. The mended fragments were prepared for ligation by linearizing with BglII restriction enzyme, dephosphorylating with alkaline phosphatase, and blunting the 5′-single-strand overhangs with Klenow. The two vectors are designed to express inserts in a mammalian system as fusions with either a secretory peptide sequence from the tissue plasmid activator gene, pCMVitPA (tPA vector) or a mouse ubiquitin subunit, pCMViUB (UB vector).

Immune analyses of infection and disease resolution have suggested a role for both humoral and cellular responses (Whitley and Miller, 2001), therefore both the tPA and UB vectors were used to drive both MHC II and MHC I presentation, respectively. The two sets of ligated products were used to transform DH5α E. coli and plated onto LB agar with ampicillin at subconfluency. These original library transformants were lifted with toothpicks and used to inoculate individual microtiter-plate cultures containing HYT freezing media (1.6% Bacto-tryptone, 1.0% Bacto-yeast extract, 85.5 mM NaCl, 36 mM K₂HPO₄, 13.2 mM KH₂PO₄, 1.7 mM Sodium citrate, 0.4 mM MgSO₄, 6.8 mM ammonium sulfate, 4.4% wt/vol glycerol) supplemented with 75 μg/mL ampicillin, and were grown overnight at 37° C. Growth and storage of the libraries as mini-cultures served to permanently maintain the original library complexity. Plasmid DNA was purified from several of the mini-cultures and analyzed to verify pathogen identity and to characterize the library. Sequence analysis established that 55% of the library inserts are HSV-1 sequences and that the remaining inserts are monkey-derived DNA, presumably from the culture cells used to propagate the viral stocks.

The plasmid-transformed bacteria were organized into twelve pools of 384 colonies transformed with the tPA vector ligation and another twelve pools of 384 colonies transformed with the UB vector ligation. A pool was comprised of four 96-well microtiter cultures. A stamping tool was used to inoculate 20×20 cm LB-carbenicillin/lincomycin agar plates with the microtiter cultures for bacterial propagation of the sublibrary plasmids. Plates were incubated at 37° C. overnight and bacterial cells harvested. The mixed-plasmid DNA samples that corresponded to each of the 24 expression library pools were purified with endotoxin-free Qiagen tip-500 column kits (QIAGEN Inc., Valencia, Calif.). The DNA quality and integrity of pool complexities were verified by spectrophotometry, enzyme digestion, and gel electrophoresis. Each of the resulting HSV insert-bearing library clones contains one randomly inserted fragment averaging 900 bp from the 152,000 bp viral genome. Since there are 384 clones in each sub-library pool, with 55% carrying HSV-1 DNA, and only 1 in 6 fragments are properly oriented and framed, one pool could express the average equivalent of 0.2 of the genome's coding sequences: (384×0.55)×900×(⅙)/152,000=0.21 expression equivalents). Together, the two intracellular targeting libraries, comprising a total of 24 sub-libraries, statistically represent 5 genome-expression-equivalents.

### Example 2

### Immunizations and Challenge-Protection Assays,

### Round 1

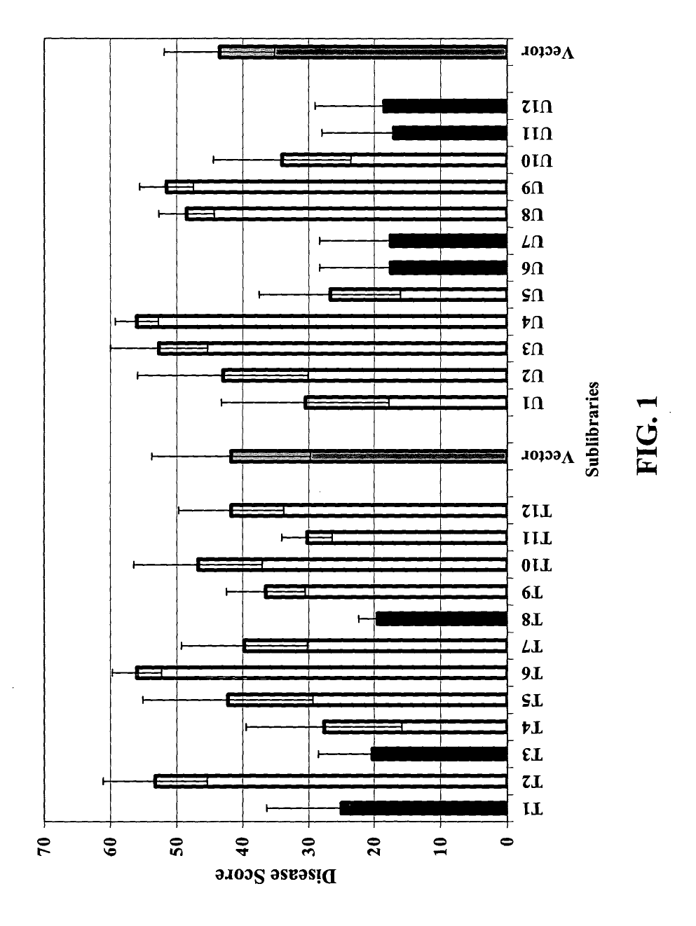

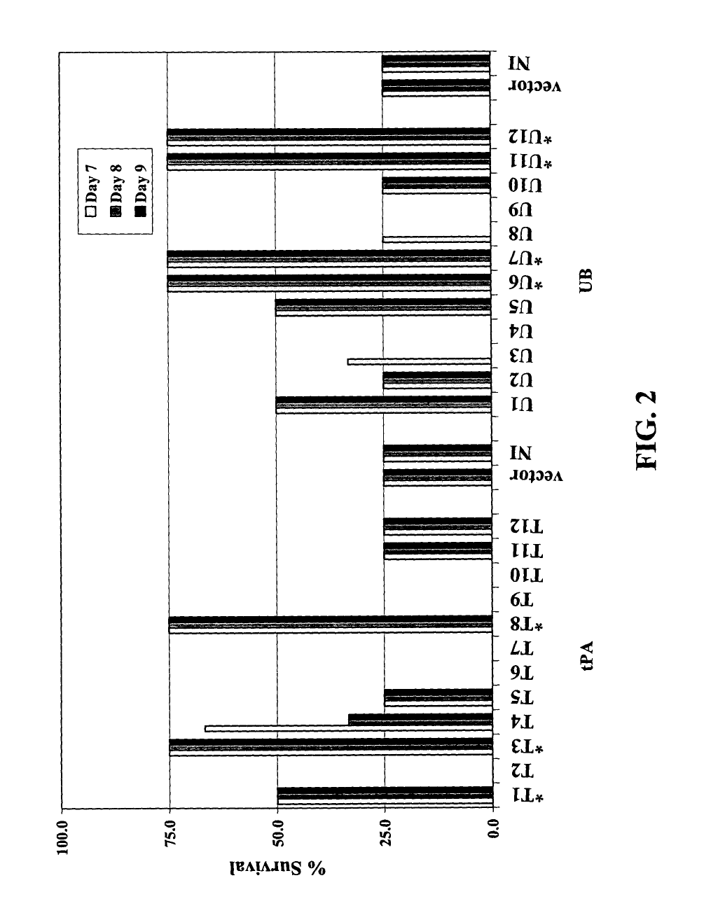

The twelve sub-library DNAs in the tPA vector and the twelve DNAs in the UB vector were each combined with a plasmid expressing murine GMCSF at 1/10 library dose in buffered saline. These inocula were intramuscularly (i.m.) injected into 24 groups of 6-week old hairless mice. Each mouse (4 per group) injected with 50 μg of pooled library plasmids and 5 μg of the genetic adjuvant GMCSF, which was evenly distributed into two quadricep and two tibialis anterior muscles. The animals were administered two boosts with the same inocula at weeks 4 and 8 post-prime then challenged with virus 2 weeks after the last immunization. Exposure to HSV-1 strain 17 syn+ was carried out by pipetting a 50 μl suspension of HSV stock containing 2×10⁵ pfu to an abraded region of shaved dermis. Both the tPA and UB library screens, using two readouts of herpes infection i) infection-induced lesions and ii) animal survival, were monitored for 14 days. Changes in the epithelium were recorded as mild, moderate, or severe. These results are described in FIG. 1. Mice with severe skin lesions and also myelitis were euthanized. FIG. 2 presents the rates of mouse survival post-challenge. Positives were scored based on both readouts: reduced lesions and increased survival relative to control animals (naïcve and irrelevant library-immunized). The two criteria were strongly correlated. Three groups from the tPA library immunizations were scored as positive, corresponding to plasmid pools T1, T3, and T8. Four groups from the UB library immunizations were identified for deconvolution, those given plasmid pools U6, U7, U11, and U12.

### Example 3

### Library Reductions, Round Two

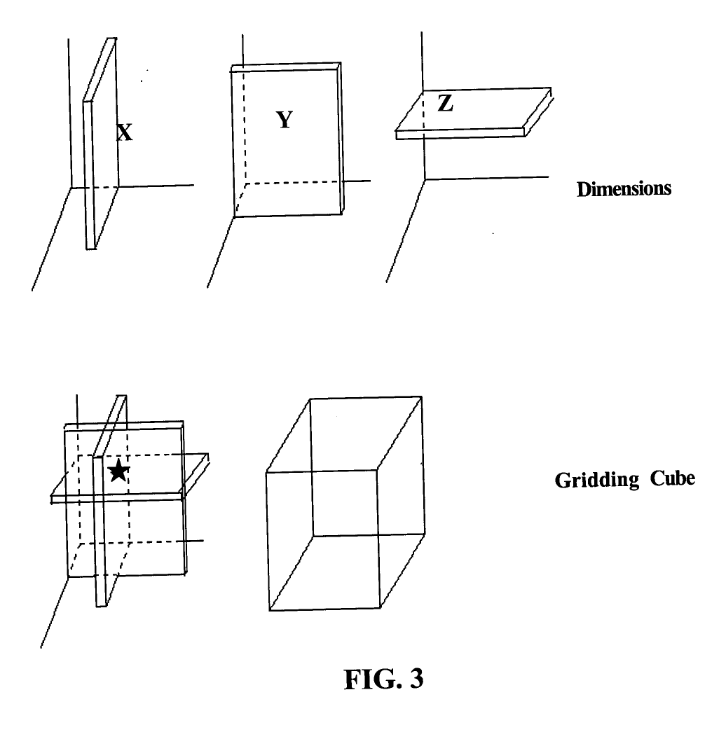

To generate the inocula for the second round of sib-testing and positive clone enrichment, the 21 microtiter culture-plates corresponding to the three positively scoring tPA groups and the four positively scoring UB groups were retrieved from the freezer stocks. Using a stamping tool, 20×20 cm LB-carbenicillin/lincomyocin agar plates were inoculated with a set of the bacterial transformants that would define the new pools of library plasmids for round 2 ELI testing. The pool compositions were designed by positioning each transformant into a virtual three-dimensional matrix, and then combining the bacteria according to the virtual planes (FIG. 3). By this pooling method, each transformant was located in three unique pools, corresponding to once in each of three dimensions. The objective was to map our protection assay data onto this grid such that a matrix analysis of the planar intersections would efficiently identified single transformants correlated with protection. The tPA grid was built with 36 groups of 100 to 200 plasmids organized into 12-X, 16-Y, and 8-Z axes. The UB grid was formed with 25 inoculation groups of 300 plasmids representing 6-X, 9-Y, 10-Z axes. Bacterial groups were propagated on the agar plates and cells were harvested. Mixed plasmid samples were purified as described above and the integrity of pool complexities were verified. The GMCSF plasmid was not included in the inocula for this and subsequent rounds of immunization. An adjuvant was deemed less important as pool complexities were reduced and the inventors preferred to avoid any possible adverse effect of inappropriate immune modulation by the cytokine expression. The mouse strain used for the challenge model was BALB/c for round 2 and 3 since the results from this strain and the hairless mice were observed to be similar. Although lesions are more easily assessed in the hairless, both strains are similarly susceptible to lethal HSV infection. Consequently, subsequent protection results obtained using the BALB/c relied on survival readouts without disease monitoring. The animals were immunized with the re-arrayed pools of library plasmids by i.m. injection (50 μg per mouse, as described for round 1), and also by gene gun delivery (1 μg per ear). The challenge procedures were similar to that described for round 1.

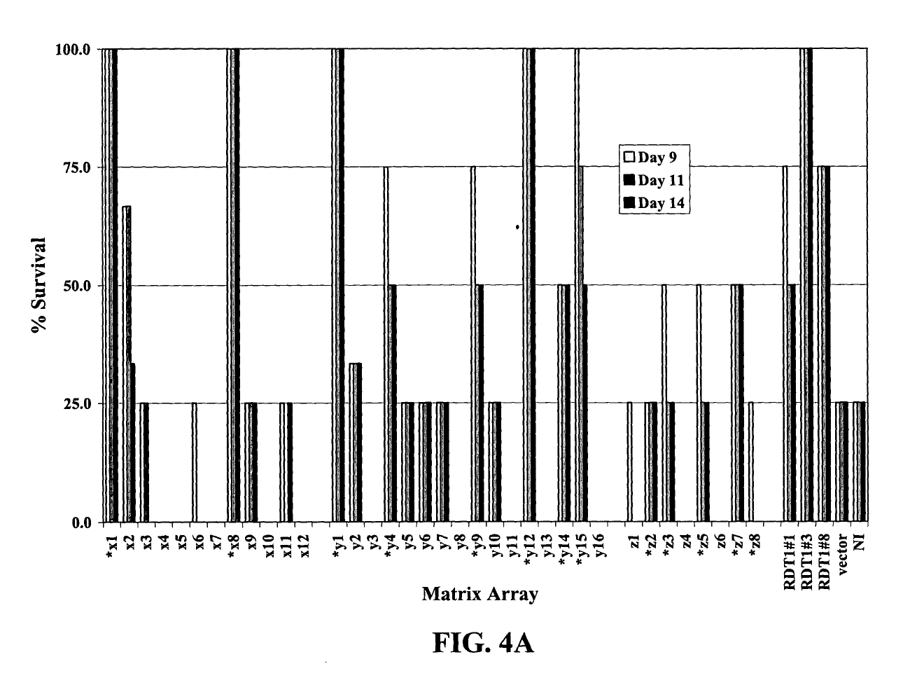

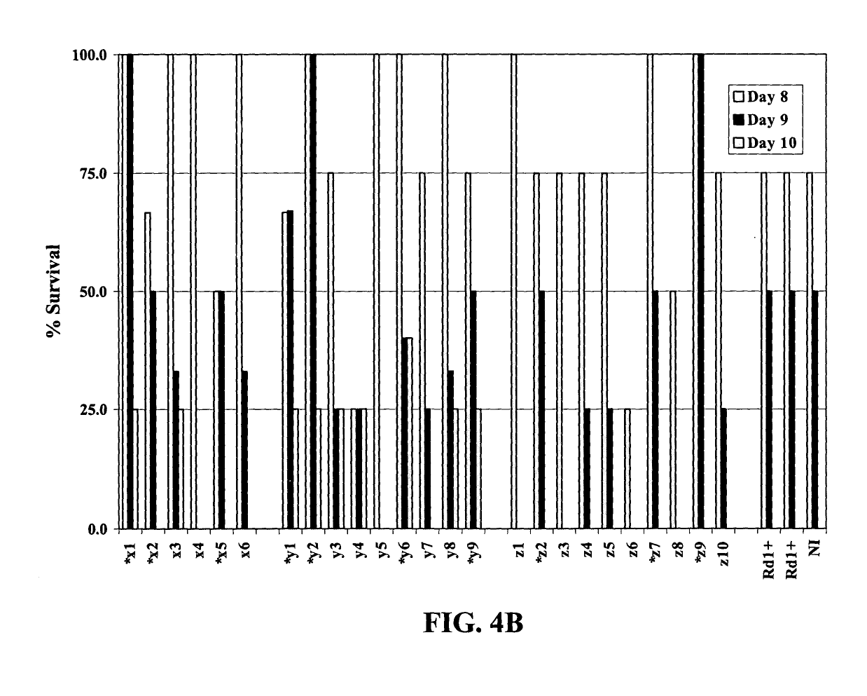

In the screen of the tPA-fused library, boosts were administered at weeks 3 and 10, and animals were exposed to virus 2 weeks after the last immunization. The challenge readout results are graphed in FIG. 4A. The positively scoring pools from round 1 were retested and again conferred protection. Negative control groups were immunized with empty vector or non-immunized (NI) mice. The top surviving test groups within each data set were chosen. Mice immunized with Z-axes pools uniformly displayed lower survival rates than those immunized with the X and Y pools, therefore scoring was less stringent for the Z axes mouse groups. The pools selected as positive corresponded to grid dimensions X1, X8, Y1, Y4, and Y9, Y12, Y14, Y15, and Z2, Z3, Z5, Z7. Their intersections indicated 48 microtiter-well transformants.

For screening the UB fusion library, mice were immunized at weeks 0, 6 and 12. The lethality results of the viral challenges, administered 3 weeks later, are graphed in FIG. 4B. Survival was monitored twice daily until 10 days post-challenge. Monitoring was not carried as long as the tPA library study because death appeared to level off by day 10 post-infection, although longer monitored may have permitted the NI to display complete death. The survival rates observed on day 9 post-infection were used to select positive groups. Again, the mice immunized with Z-axes pools uniformly displayed lower survival rates. The best surviving groups within each data set were chosen. These groups were immunized with pools of plamids representing matrix planes X1, X2, X5, and Y1, Y2, Y6, Y9, and Z2, Z7, Z9. Their intersections indicated that 90 microtiter-well transformants from the originally designed grid were responsible for the observed improvements in survival.

### Example 4

### Reduction to Individual Antigen-Encoding Clones

Each of the library transformants designated by the matrix cross-hairs was individually propagated in liquid culture and the plasmid was purified using a small-scale alkaline lysis kit method (Qiagen, Turbo-preps). Sequencing reactions were performed with primers that hybridize immediately upstream and downstream of the library insert cloning site. Analyses of the sequence data were used to identify inserts that encoded properly fused HSV-1 open-reading-frames (ORFs) greater than 50 amino acids (aa) in length.

From the group of 48 tPA peptide-fused library clones, 21 carried contaminating mammalian-DNA inserts and another 26 carried non-coding HSV-1 DNA. Six clones encoded HSV-1 ORFs that encoded fragments from the following six proteins:

1. US6, glycoprotein D (gD), currently studied as a vaccine candidate. The gD library insert identified in the screen was 1385 bp, and spanned the full-length gene.

2. US3, a serine/threonine protein kinase.

3. UL17, a viral DNA cleavage and packaging protein.

4. UL50, a dUTPase. The insert encodes an open-reading frame greater than 50 aa however it is not in the predicted coding frame.

5. US8, glycoprotein E (gE), known to inhibit IgG-mediated immune responses.

6. UL28, viral DNA cleavage and packaging protein and a transport protein.

From the group of 98 UB-fused library clones, 27 carried contaminating mammalian-DNA inserts and 25 were HSV-1 inserts but did not encode an HSV-1 protein fragment. Eight plasmids encoded HSV-1 ORFs corresponding to one or more fragments of the following six proteins:

1. Anti-sense of UL29/ICP-8

2. UL53, glycoprotein K (gK).

3. UL27, glycoprotein B (gB), currently studied as a vaccine candidate.

4. UL36, the very large tegument protein.

5. UL29/ICP-8, major single-stranded DNA-binding protein.

6. UL24, a replication protein.

Sequencing revealed that three unique library clones carried inserts corresponding to three different regions of the approximately 10 kilobase UL36 gene. Two of these encoded UL36 fragments and one of these was scored as positive. Two ORFs corresponded to the UL29 gene. One of these encoded a fragment of UL29 and the other ORF appears to be fortuitous since the UL29 coding sequence was fused in an inverted orientation.

### Example 5

### Protection Analysis with Individual Library

### Clones, RELI Round 3

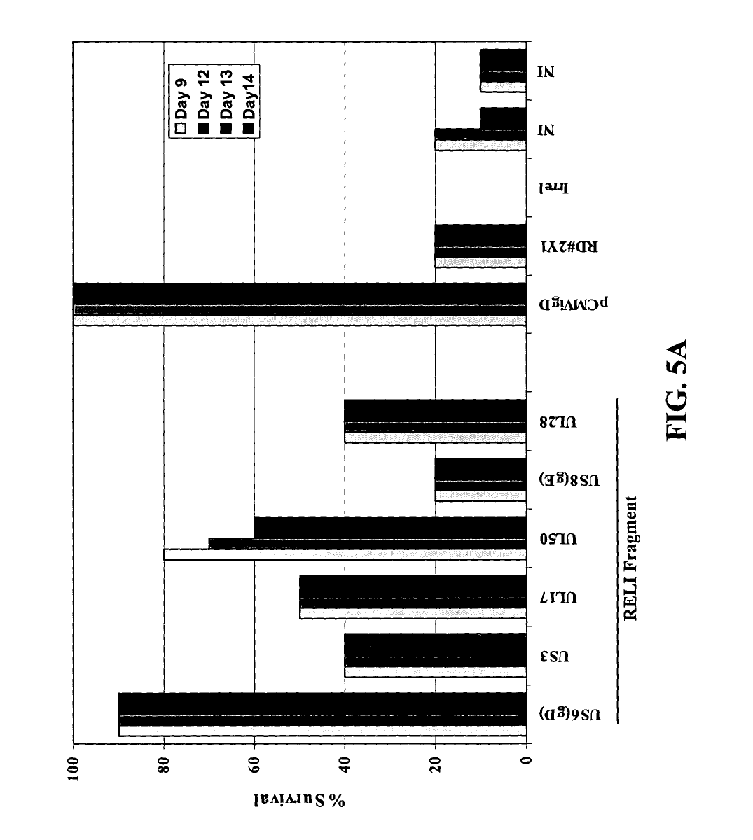

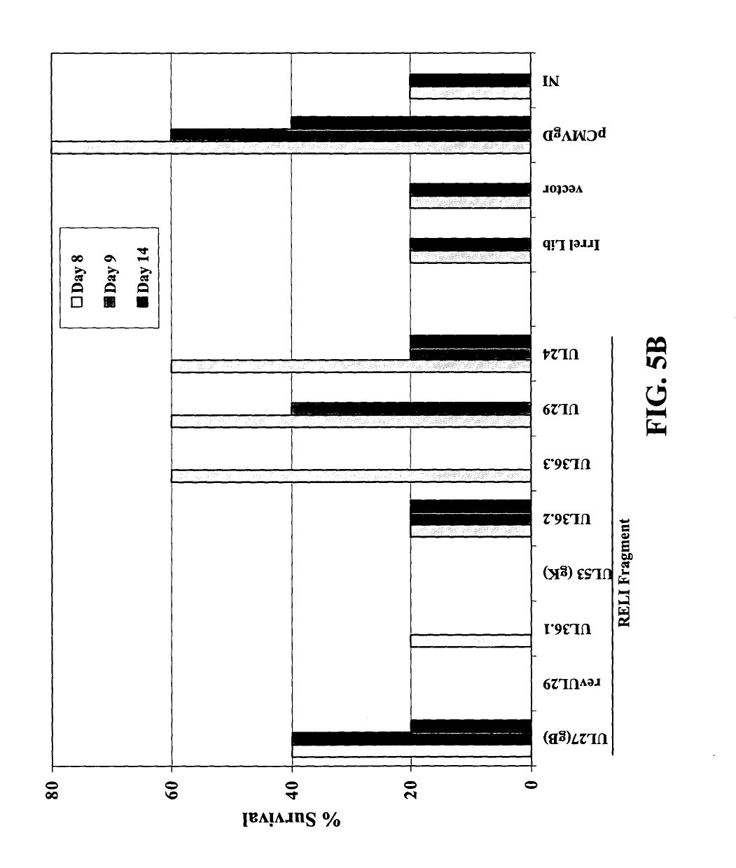

Stock bacterial cultures carrying each of tPA and UB library clones indicated above were grown in liquid culture by standard methods and the plasmids were purified with Qiagen endotoxin-free kits. Less library plasmid was used for the single clone inoculations, since the dose of each one was high relative to the earlier rounds. If the total amount of DNA in an inoculum is maintained, then the dose of any one antigen increases as the complexity of the mixture decreases. For round-3 of the tPA screen, inocula for vaccination were prepared by diluting each library plasmid with an equal amount of pUC118, as non-specific carrier DNA to facilitate delivery. In particular, BALB/c mice were injected i.m. with 50 μg of DNA, comprised of 25 μg of one of the protection candidates and 25 μg of pUC118. They were simultaneously administered two 1 μg DNA shots with the gene gun, each comprised of 0.5 μg of same vaccine candidate with 0.5 μg pUC118. The animals were boosted with the same inocula at weeks 5 and 9. Three weeks following the last boost, vaccinated animals were challenged with HSV-1 strain 17 syn⁺. Unfortunately the viral stock was less virulent than anticipated, evidenced by survival of the unimmunized control mice. The animals were re-challenged two-weeks later with a fresh stock of titered HSV-1, and survival was monitored and recorded for 14 days. To confirm that the second challenge had not altered the readout, the tPA-library round-3 study was repeated and similar results were obtained. The survival results are shown in FIG. 5A. Immunization with five of the six clones led to survival rates that were at least twice as high as the negative control groups (non-immunized and irrelevant-antigen immunized). These clones encode gD (US6), a serine/threonine kinase (US3), two viral packaging proteins (UL17 and UL28), and UL50. A positively scored pool from round-2 did not perform as well as the single clone inocula in this study. This may be attributable to the more severe conditions of a double challenge with no adjuvant, and/or its several-hundred fold complexity, and therefore dilution, relative to the single plasmid inocula.

The UB fusion vector is designed to facilitate proteasome processing and MHC I-stimulated immune responses. The inventors have previously observed that, unlike antibody responses, cellular responses can decline once the optimal dose has been surpassed. Therefore, the inventors chose to imitate the gene dose of each antigen within the sublibrary pools by mixing the single plasmids with pUC 118 into a 200-fold dilution (0.25 μg i.m. and 0.005 μg per gene gun shot). Mice were primed individually with the eight ORF-containing clones, and then boosted twice at weeks 5 and 11 with the same single plasmid inocula. Vaccinated animals were challenged 2 weeks later with HSV1 syn17⁺ as described above. These results showed that the inoculum was not sufficiently lethal. Fresh HSV stocks were prepared and titered, and the challenge was repeated 6 weeks later. Survival was monitored and recorded for 14 days. Presumably as a result of this double challenge, protection levels were generally lower than previously observed. Namely, even the positive control gD-expressing plasmid (pCMVigD), delivered at a full (undiluted) dose, provided only partial protection. The survival percentages on representative days 8, 9, and 14 are plotted in FIG. 5B. Immunization with four clones led to extended survival relative to the non-immunized group. These clones encode fragments of UL27 (gB), UL36.2, UL29, UL24.

### Example 6

### Comparative Protection Assays of RELI-Identified

### HSV-1 Gene Fragments

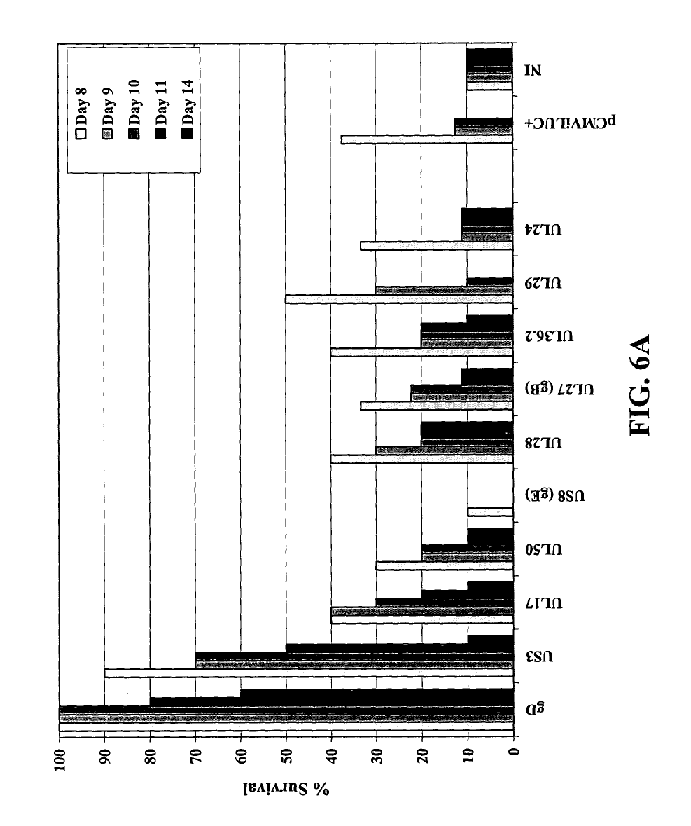

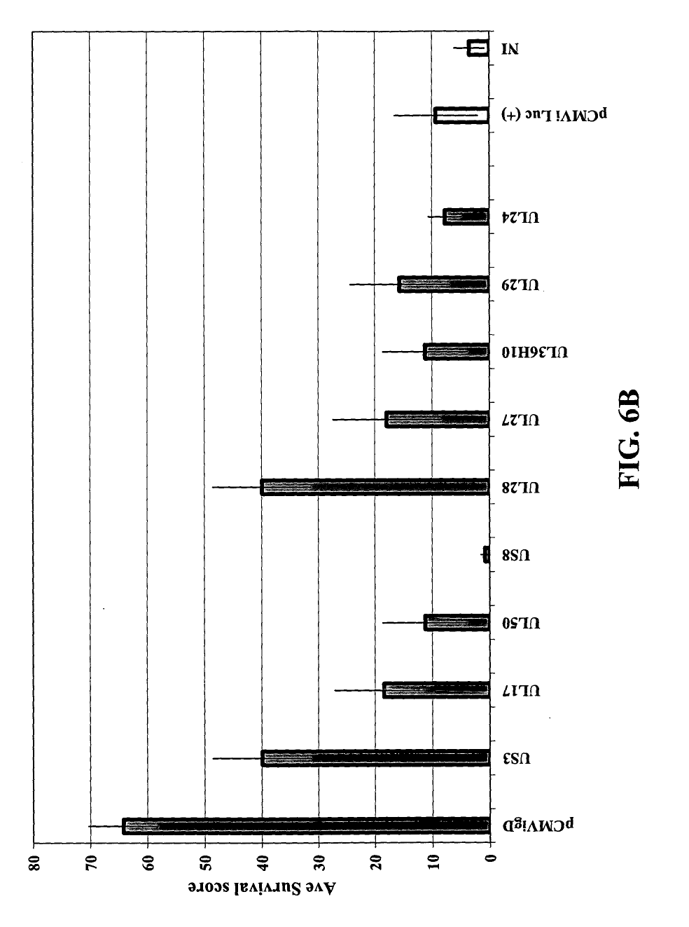

This study was conducted in order to assess the relative levels of protection conferred by the gene vaccine candidates. All ten library clones that had been identified in the tPA and UB RELI library screens were retested using the original gene-fragment constructs. The plasmids were delivered by gene-gun (2×1 μg) and i.m. (50 μg) routes, into groups of 10 mice each. Several of these gene-fragment inocula led to extended mouse survival, although none performed better than the full-length gD construct. In particular, fragments of UL17, US3, UL50, UL28, and UL36 (UL36.2) showed some protection relative to the non-immunized control mice. These results are presented in FIGS. 6A and 6B. In FIG. 6A, the percentage of each group surviving at representative days 8 through 11 and the endpoint day 14 are shown. In FIG. 6B, an average survival score has been calculated for each group, and plotted alongside the positive and negative control groups, which were immunized with pCMVigD, pCMViLUC, respectively or NI. A score was calculated for each animal by summing the day-numbers post-exposure (days 8 through 14) during which the animal lived. An average score and standard error was calculated for the group and used for graphing. The results show that immunization with US3, UL17, UL28, UL27 (gB), and UL29 generated protection scores with non-overlapping standard errors to that of the NI controls.

### Example 7

### Analysis of Candidate Antigens for HSV-1 Vaccines

By utilizing RELI and two intracellular targeting genetic immunization vectors, four viral genes were newly identified as vaccine candidates for including in a subunit-based Herpesvirus vaccine. The libraries comprising round 1 were screened in the presence of GMCSF, while the inocula in rounds 2 and 3 were tested without adjuvant. When retested, the positively scored sub-libraries from round 1 were found also to be protective without GMCSF. The immunization route in round 1 was i.m. injection only, and subsequent rounds included both injection and gene gun delivery. Also the first round was done in Hairless mice while the subsequent rounds were conducted in BALB/c mice. These differences between rounds indicate that the output candidates were capable of conferring protection independent of GMCSF co-delivery, with or without gene gun delivery, and in at least two different mouse model strains.

In addition to the four unique candidates, both of the two major antigens currently studied as vaccine candidates were identified. In particular, screening the tPA fusion library yielded the full length glycoprotein D gene, and screening the UB fusion library yielded an expressed fragment of the glycoprotein B gene. The fragment carried on this library clone encodes a determinant that has been shown to be immunogenic in infected individuals. The output of known vaccine candidates by the ELI process supports the validity of the unbiased method and suggests the utility of the other output antigens.

None of the new vaccine candidates from the RELI screens are predominantly surface proteins. Instead enzymes, nuclear proteins, and cytoplasmically-located proteins were discovered. For example, a new candidate from the tPA library screen expresses an N-terminal fragment of US3, serine/threonine protein kinase. In both HSV-1 and 2, the US3 gene is required for the characteristic herpes virus-induced blockage of programmed cell death. Interestingly, one of the other two genes thought to block apoptosis is gD (Whitley and Roizman, 2001). US3-deficient mutant strains replicate normally but are highly attenuated. Despite the reduced virulence these mutants display enhanced immune activity, suggesting a role for US3 in suppressing host immune responses (Inagaki-Ohara et al., 2001). In cytomegalovirus, US3 has been shown to delay the presentation of viral antigens to cytotoxic T cells (Jones et al., 1996). In a screen for human T cell epitopes, a 15 aa peptide mapping to US3 has been identified as stimulating CD4 T cells in an in vitro proliferation assay (U.S. Patent Application 20020090610). To our knowledge, the US3 protein kinase has not been previously predicted to be, or tested as, a vaccine candidate. Two of the other candidates from the tPA-fusion library screen encode fragments of proteins involved in viral DNA cleavage and genome packaging, UL17 and UL28. To our knowledge, neither has been previously implicated as protective antigens. A new candidate derived from the UB library screen is UL29. The UL29 gene product is ICP-8, a single-stranded DNA binding protein required for viral replication. It appears to be involved in recruitment of the helicase-primase complex to DNA lesions (Carrington-Lawrence et.al, 2003). Mutant HSV-2 deficient in UL29 are defective in DNA synthesis and replication (Da Costa et al., 2000). In cytomegalovirus (CMV), the UL36-38 complex synergizes with the US3 protein to regulate transcription of the heat shock protein 70 gene of the host.

Table 2 provides the sequences and summarizes the lengths of each of the HSV random library fragments that conferred mice protection against challenge in the comparative study. The length of the gene-encoding portion within the random fragment, and the size of the full gene are given. In Table 3, the pooling history of these library clones during the library reduction is described.

TABLE 2
The HSV-1 vaccine candidates identified by RELI.

| | Library | | Coding | Full length | |
|---|---|---|---|---|---|
| Gene | Insert | Insert SEQ ID No. | fragment | gene | Gene SEQ ID No. |
| US6(gD) | 1381 | SEQ ID NO: 111 | 1185 | 1185 | SEQ ID NO: 115 |
| US3 | 974 | SEQ ID NO: 103 | 969 | 1446 | SEQ ID NO: 105 |
| UL17 | 1425 | SEQ ID NO: 33 | 558 | 2112 | SEQ ID NO: 39 |
| UL28 | 1815 | SEQ ID NO: 57 | 1815 | 2358 | SEQ ID NO: 63 |
| UL27 (gB) | 683 | SEQ ID NO: 53 | 681 | 2715 | SEQ ID NO: 55 |
| UL29 | 514 | SEQ ID NO: 65 | 513 | 3591 | SEQ ID NO: 67 |

TABLE 3
Resident pools of the RELI candidates

| Derivative Gene | Round 1 pool | Round 2 pools |
|---|---|---|
| US6 (gD) | T3 | T: X1, Y1, Z7 |
| US3 | T3 | T: X1, Y14, Z2 |
| UL17 | T8 | T: X1, Y9, Z3 |
| UL28 | T8 | T: X8, Y14, Z7 |
| UL27 (gB) | U7 | U: X2, Y6, Z9 |
| UL29 | U12 | U: X1, Y6, Z7 |

Table 4 presents the amino acid similarities and identities of the products encoded by the ELI-identified HSV-1 gene fragments to their homologs in a selection of other herpesviruses. These sequence comparisons may indicate that the HSV-1 homologs could carry protective capacities. For example, gD of BHV has been shown to be protective against BHV, as is its homologue from HSV-1 and HSV-2. Notably, a number of the RELI candidates display herpesvirus similarities/identities that are higher than that of gD. The relatedness also suggests that vaccination with genes or gene products from one virus might heterologously protect against exposure to a different herpesvirus.

TABLE 4
Examples of Percent Similarities/Identities of
RELI hits to Herpesvirus homologs.

| Gene | | | | | | |
|---|---|---|---|---|---|---|
| fragment | HSV2 | VZV | BHV | EHV | CMV | CHV |
| gD | 82/88 | 25/44 | 27/39 | 23/40 | 30/33 | 58/72 |
| US3 | 70/79 | 44/61 | 44/62 | 34/55 | 26/36 | 51/64 |
| UL17 | 84/90 | 31/53 | 34/48 | 31/51 | 33/43 | 69/79 |
| UL28 | 88/91 | 46/62 | 51/64 | 52/67 | 22/41 | 81/87 |
| gB | 90/95 | 45/67 | 45/64 | 42/58 | 26/42 | 77/86 |
| UL29 | 97/98 | 48/63 | 53/67 | 55/71 | 26/40 | 88/91 |

### Example 8

### A DELI Screen: Construction of an HSV-1 Gene Library

Genomic DNA from the MacIntryre strain of HSV-1 was purified from cultured green monkey kidney cells (VERO-E6). The genomic DNA itself would be used as template for polymerase chain reactions. A backup source of template was generated by cloning the genomic DNA into plasmids. In this state, the DNA would have different characteristics (e.g. topology) and be a renewable resource. The two libraries described in example 1 for RELI were also used as an alternative plasmid template for DELI.

To build an expression library of all HSV-1 genes, a set of two oligonucleotides (oligos) were designed that correspond to the 5′ and 3′ end sequences of each open-reading-frame (ORF) to provide for sequence-directed PCR-amplification of the HSV-1 coding sequences. Each primer was designed to optimize the probability of successful hybridization and to roughly match the melting temperature (Tₘ) of its primer pair. Accommodations were made for repetitive sequences, GC-content, melting temperature, product length, and LEE linking. Genes longer than 1,500 bp were split into sub-gene fragments. To facilitate the attachment of expression elements to the ORFs, each primer was designed with a 15 base deoxyuracil (dU)-containing stretch at its 5′ end, followed by approximately 20 nucleotides of ORF-specific sequence. The dU stretch is comprised a repeated triplet sequence, which contains a dU phosphoramidite, and renders the region sensitive to uracil-DNA-glycosylase (UDG) degradation. The purpose of including this sequence is to generate a single-stranded region by degrading the 5′ stretch and creating a 3′overhang. The sequences of the dU stretches are designed to prevent the ORF from self-annealing, but permit complementary annealing to promoter and terminator expression fragments. Each oligo was designed to ensure that the coding frame of the HSV-1 polypeptide would be maintained. Primer sets to amplify 126 ORFs that would encode for the 77 HSV-1 genes were synthesized on a MerMade IV™ instrument in 96-well formats. The 35 to 37 base oligo products were evaluated for quality by gel electrophoresis, and evaluated for yield by fluorimetry.

The dU-containing oligo stocks were diluted to 10 μm then combined into ORF primer sets. A reaction master-mix was prepared to PCR-amplify each ORF as follows:

| | | |
|---|---|---|
| 10X PCR buffer with MgCl₂ (Promega), | 10 | μl |
| 2.5 mM dNTPs | 5 | μl |
| dH₂0 | 55.8 | μl |
| HSV-1 genomic DNA (1.2 ng/ul) | 8.2 | μl |
| Taq polymerase (Promega) | 1 | μl |

ORF-specific primers were separately added to each microtiter well:

dU primer pair (10 μm) 20 μl

Reactions were incubated in a thermocycler (Perkin-Elmer) by the following program:

| | |
|---|---|
| 96° C., melting | 2 min |
| 94° C., melting | 30 sec |
| 55° C., annealing | 30 sec |
| 72° C., polymerizing | 1 min, 30 sec |
| Cycle 34 times, then 72° C. | 10 min |

The high GC-content of the HSV genome (69%) and number of repetitive sequences are believed to have led to the need for extensive PCR testings. Reactions that did not amplify with sufficient specificity or yield were re-prepared and run in a Robocylcer (Strategene, La Jolla, Calif.) temperature gradient program. Optimal amplifications of the 126 primer sets were found to require eight different annealing temperatures that vary from 33° C. to 63° C. In addition, optimal amplification of the ORFs encoding a subset of ORFs, such as the UL36 gene and a portion of the UL29 and UL27 genes, required the addition of 6% DMSO to the reactions. The DMSO-containing samples were the only reactions programmed at the lowest annealing temperature, 33° C. Once appropriate conditions were identified, multiple reactions were prepared to amplify sufficient quantities of each ORF. Identical products were combined and were precipitated by adding 0.3 M sodium acetate and 3 volumes of ethanol. Products were resuspended in water, and a sample (5/100) of each PCR product was analyzed by agarose gel electrophoresis alongside a quantitated 100 bp DNA standard ladder (Promega, Madison, Wis.). Another sample (1/100) was removed to measure DNA concentration with pico-green dye in a Tecan plate-reader (Tecan, Research Triangle Park, N.C.) by fluorimetry using a kinetic measurement program.

### Example 9

### Arraying of an HSV-1 ORF Library According to Cubic Designations

The quality- and quantity-controlled ORFs were arrayed into 75 pools (25 X's, 25 Y's, 25 Z's) of 5 ORFs according to their computer-assigned location with in virtual 25×25×25 grid. Each new pool represented the constituents of the x, y, and z planes of the computer-derived three-dimensional matrix. Since each ORF holds a position in all three dimensions, each ORF is contained in three independent pools for subsequent testing. The pooling was accomplished robotically using a BioMek (Beckman, Brea, Calif.) instrument. A program was written that imported the PCR product names and concentrations, and then distributed the each product into three of 75 wells (representing 25 X, 25 Y, and 25 Z pools) such that all ORFs were present at equal molar amounts in each pool. Since the product lengths varied, the total amounts of DNA per well varied from 2.6 to 3.9 μg. The volumes of samples in the wells were raised to a common 150 μl with dH₂O to prepare for the uracil DNA-glycosylase reactions:

| | | |
|---|---|---|
| PCR products | 150 | μl |
| 10x UDG buffer (NEB) | 17.3 | μl |
| UDG | 6 | μl (6 units) |

The reactions were incubated at 37° C. for 40 minutes then the enzyme was inactivated at 65° C. for 10 minutes. The resulting products will carry 15 base single-stranded stretches at both ends. To purify the samples, 200 μl of Magnasil DNA-binding beads (Promega, Madison, Wis.) were added and the samples were vortexed for 30 minutes. After settling, the supernatant was transferred to a separate tube and purification was repeated with 200 μl of fresh beads. Wash solution was added to the beads and vortexed as directed. Beads were washed in 80% ethanol as directed, then dried. Elution buffer was added to beads to recover the PCR products. Volumes were reduced to 50 μl by lyophilization.

### Example 10

### Preparation of the Arrayed Library for Gene Expression

Based on numerous genetic immunization studies using both plasmid and LEE based antigen expression, the inventors arrived at pair of expression elements that reliably performed well. The promoter element is a PCR product comprised of the cytomegalovirus immediate early gene promoter, the chimeric intron of pCI, and one of two fusion peptides for intracellular targeting the antigen. The two fusions, as described earlier, are designed to favor either MHC II or MHC I presentation by using i) a secretory leader sequence from human α1-antitrypsin (LS) and ii) a short ubiquitin subunit sequence (UB). The terminator (GHterm) is a PCR product comprised of the human growth hormone transcription termination sequence. To facilitate consistency, these three expression elements were prepared in large batches, with the following 100 μl standard-reaction master-mix:

| | |
|---|---|
| 10x PCR buffer with MgCl₂ (Promega) | 10 μl |
| 2.5 mM dNTPS | 5 μl |
| ddH₂O | to final volume of 100 μl |
| Taq (5 units/μl) (Promega) | 1 μl |

The mix was divided into three parts and different sets of template and primer were added to each:

| For the LS promoter-fusion element (product size is 1.2 kb): | | |
|---|---|---|
| Plasmid template pCMViLS | 50 | ng |
| CMV Fprimer151 | 1 | μg |
| LS dU Rprimer | 1.5 | μg |
| For the UB promoter-fusion element (product size is 1.34 kb): | | |
| Plasmid template pCMViUB | 50 | ng |
| CMV Fprimer151 | 1 | μg |
| UB dU Rprimer | 1.5 | μg |
| For the GH terminator element (product size is 0.61 kb): | | |
| Plasmid template pCMVi | 50 | ng |
| GHterm dU Fprimer | 1 | μg |
| GHterm Rprimer1590 | 1.5 | μg |

The plasmid templates were genetic immunization vectors without any coding sequences (no insert) that contained either the leader sequence or ubiquitin sequence and the human growth hormone gene terminator. These were linearized by digestion with PvuI restriction enzyme to facilitate PCR-amplification. In each expression element primer set, one primer contains a dU stretch and one primer does not. The sequences of these oligo primers have been previously described (Sykes and Johnston, 1999). For the ORF primer sets, both primers contain dU stretches. Reactions were incubated in a thermocycler (Perkin-Elmer, Boston Mass.) by the following program:

| | |
|---|---|
| 96° C., melting | 3 min |
| T*, annealing | 1 min, 15 sec |
| 72° C., polymerizing | 1 min, 30 sec |
| 94° C., melting | 45 sec |
| T*, annealing | 1 min, 15 sec |
| 72° C., polymerizing | 1 min, 30 sec |
| Cycle 34 times, then 72° C. | 10 min |

*Optimal annealing temperatures (T) varied between the elements as follows: 44-55° C. for LS promoter-fusion, 54-55° C. for UB promoter-fusion, 44-65° C. for terminator.

Multiple 100 μl reactions are prepared at once, and then collected for purification. Sodium acetate is added to a final concentration of 0.3 M, and then the samples are extracted one time with an equal volume of phenol/chloroform. The aqueous was removed into a fresh tube then ethanol precipitated. The pellets were resuspended in water at one-fourth their original volume. The elements were analyzed by gel electrophoresis and concentrations were determined by flurometry.

The linear expression elements (LEEs) were created by combining the two promoter-fusion elements and the terminator element into each of the pooled ORFs so as to provide equivalent molar ratios of expression elements to ORFs. In particular the molar ratios of the two promoter-fusions to ORF to terminator was calculated so as to be 0.5:0.5:1:1.

ORFs (approximately 3.75 μg in 50 μl)

| | |
|---|---|
| 10x Annealing buffer | 10 μl |
| 1.25 μg CMViUB | 6.25 μl |
| 1.25 μg CMViLS | 6.94 μl |
| 1.25 μg GHterm | 4.2 μl |

The linking reactions were incubated at 95° C. for 5 minutes then transferred to 65° C. After 1 minute to cool sample, 2M KCl (25.8 μl) was added to a final concentration of 0.5 M. Samples were incubated at 65° C. for 10 minutes, then 37° C. for 15 minutes, and then 25° C. for 10 minutes. To assess linking efficiency 1 μl was removed, diluted 5-fold into TE and loading dye, and then electrophoresed at low voltage on a 0.7% agarose gel.

### Example 11

### Preparation of the Arrayed LEE Expression

### Library for Direct Mouse Inoculation

Inocula for animal immunizations were made by mixing the expression element-linked ORFs (approximately 7.5 μg in 100 μl) with linearized plasmid DNA (pUC118) to total 30 μg of DNA. The EcoRI-digested pUC118 filler served as carrier for more efficient gold precipitation (see below). For each HSV gene pool inoculum, 30 gene-gun doses (bullets) were prepared, such that each shot delivered 250 ng of HSV DNA along with 750 ng of carrier. Gold microparticles with diameters ranging from 1-3 μm (Degusa Inc.) were weighed out dry into multiple microfuge tubes at 75 mg per tube. Particles were washed with approximately 1 ml ddH₂O then removed, cleaned with approximately 1 ml 100% ethanol then removed, and then finally resuspended in 1.25 ml of ddH₂O to obtain a slurry of gold at 60 mg/ml. The slurry was aliquotted at 225 μl per each of 75 microfuge tubes. The tubes were gently spun to pellet gold and then the ddH₂O was removed. To each of the tubes, a 100 μl linking reaction and 22.5 μg of pUC118 was added. The DNA/gold slurry was vortexed and 1 volume (130 μl) of 2.5 M CaCl₂, pH5.2 was added. While vortexing, 1/10 vol (26 μl) of 1 M spermidine (free base) was added. The samples were allowed to precipitate on the gold microparticles for 15 min at room temperature, and then spun at room temperature for 1 minute. Supernatants were removed and the gold was washed with 70%, then 100% ethanol three times. The washed samples were combined with 1.8 ml fresh, very dry 100% ethanol and then dried overnight in a dessicator. Gene-gun bullets were prepared as per Helios instructions (BioRad, Inc., Hercules Calif.). Briefly, each 1.8 ml sample was drawn into a syringe and injected into dry plastic tubing that fixed onto a rotating station. DNA attached gold was dried onto the inner surface of the tubing by blowing nitrogen through it. The inventors have adapted the station to accommodate 8 samples at once. Up to 30 bullets were obtained from each batch, and one was used for analysis. A bullet was placed in a tube with TE and loading dye. The solution was then loaded onto an agarose gel for analysis. Prepared bullets were stored in a dessicator until used for immunizations.

### Example 12

### Mouse Immunizations and HSV-1 Challenge-Protection

### Assays

The 75 pools of LEEs expressing 5 HSV ORF and controls were administered to groups of 4 BALB/c mice, as three sets of 25 dimensionally-defined test pools. Positive control groups received a plasmid or LEE expressing the known vaccine candidate glycoprotein D₁ (gD) and negative control groups were non-immunized (NI). Each mouse received a total of 2 μg of DNA delivered on gold microprojectiles with a Helios gene gun. The immunizations were distributed as two 1 μg doses into the skin of the mouse ears. Each test dose was comprised of 250 ng of HSV-1 DNA (and therefore 50 ng of each individual ORF) and 750 ng of pUC118 DNA as filler. Each positive control dose was comprised of 250 ng of pCMVigD or LEE-gD, and 750 ng of pUC118. The animals, were administered two boosts with the same inocula at weeks 4 and 8 post-prime then challenged with virus 3 weeks after the last immunization. Exposure to HSV-1 pathogenic strain 17 syn⁺ was carried out by pipetting a 50 μl suspension of viral stock containing 2×10⁵ plaque-forming-units to an abraded region of shaved dermis. Survival was monitored for 12 to 15 days; disease-induced death began on day 6 and continued through day 12 post-exposure.

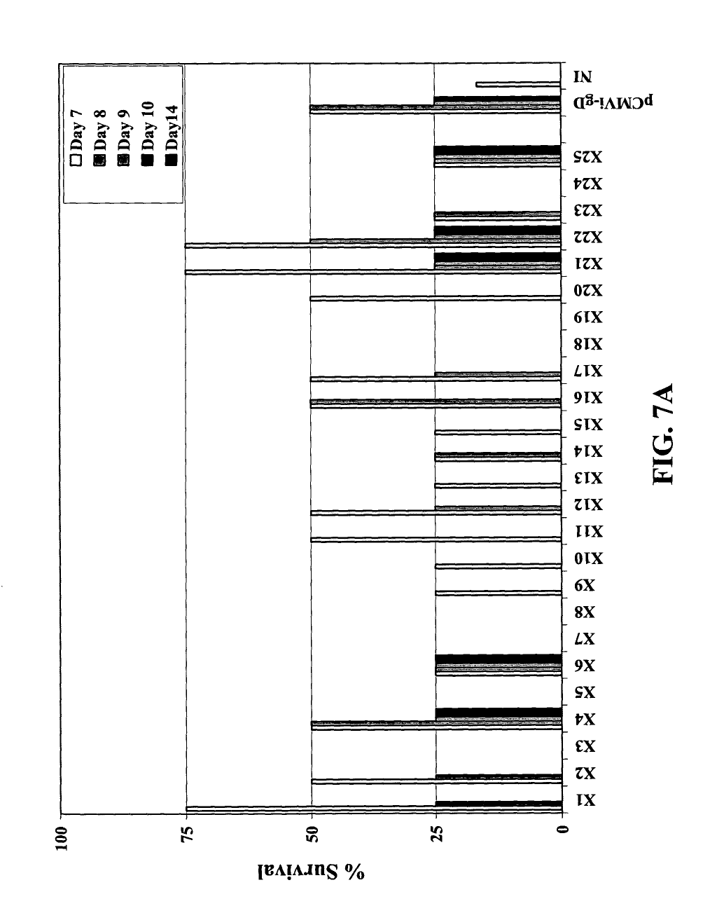

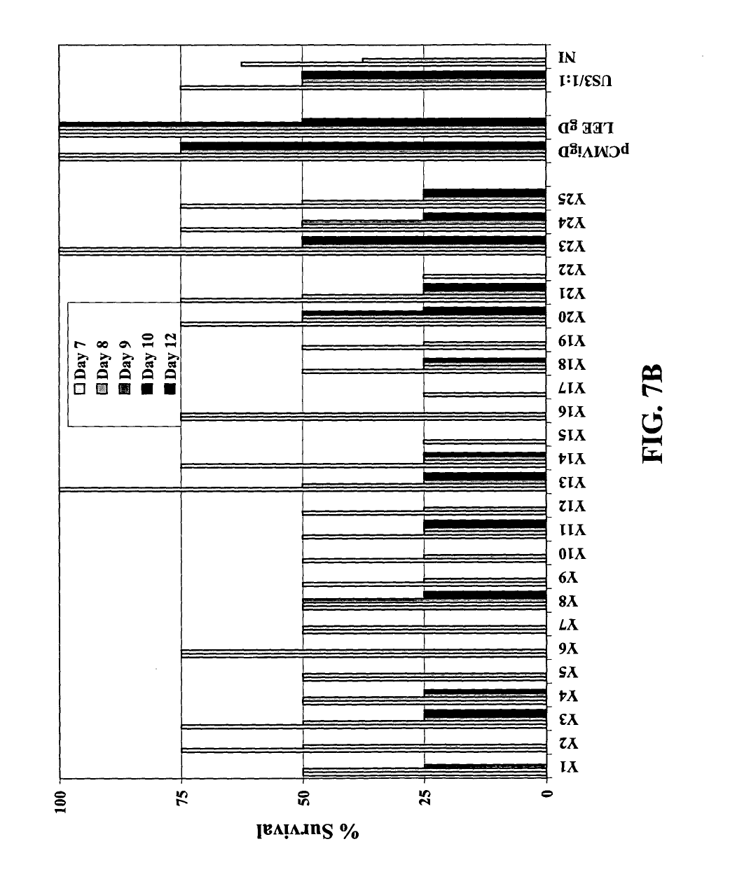

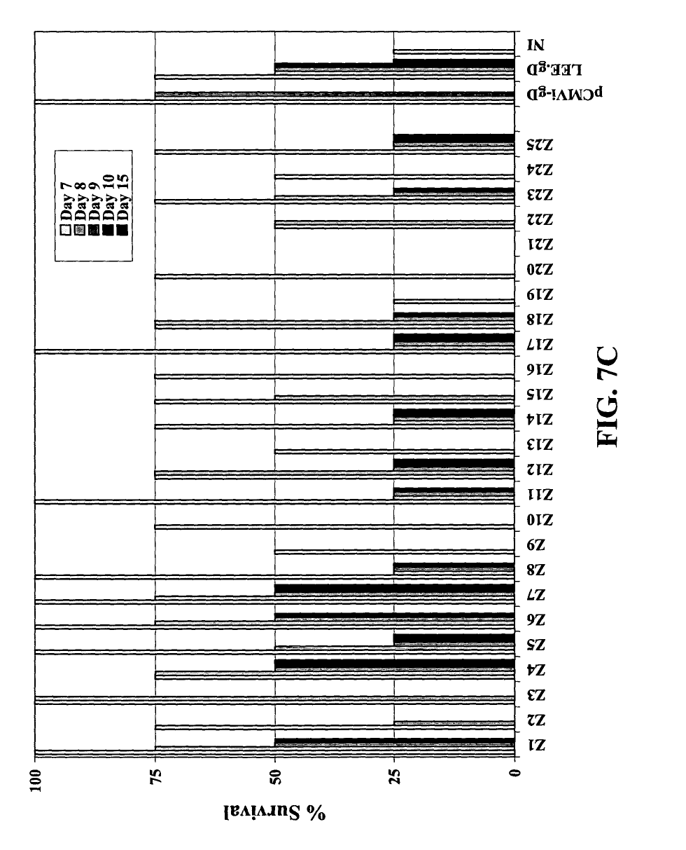

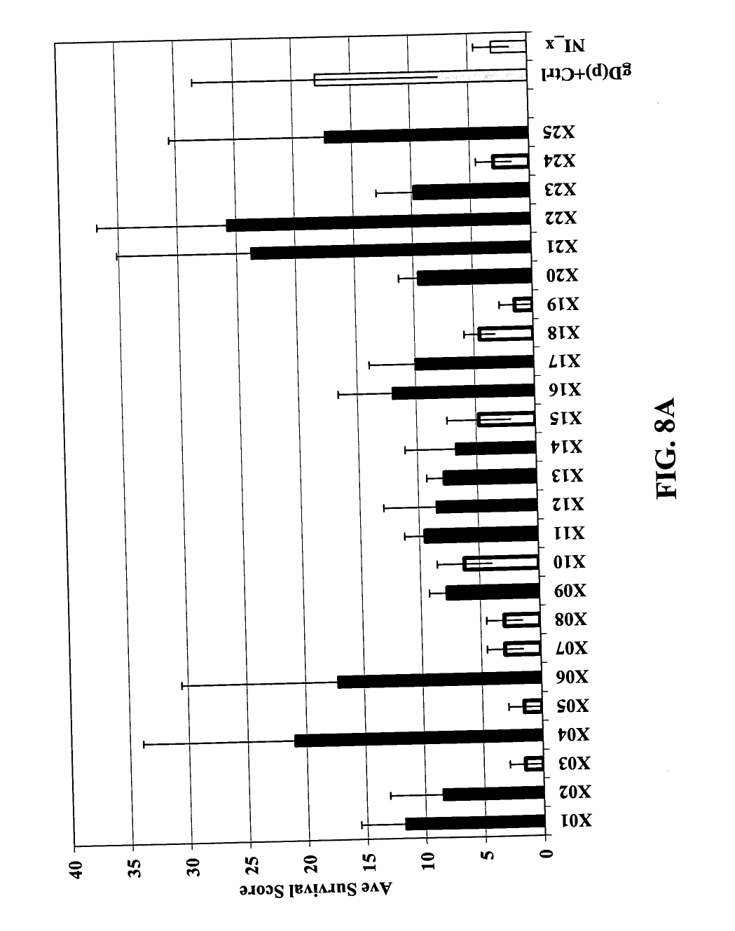

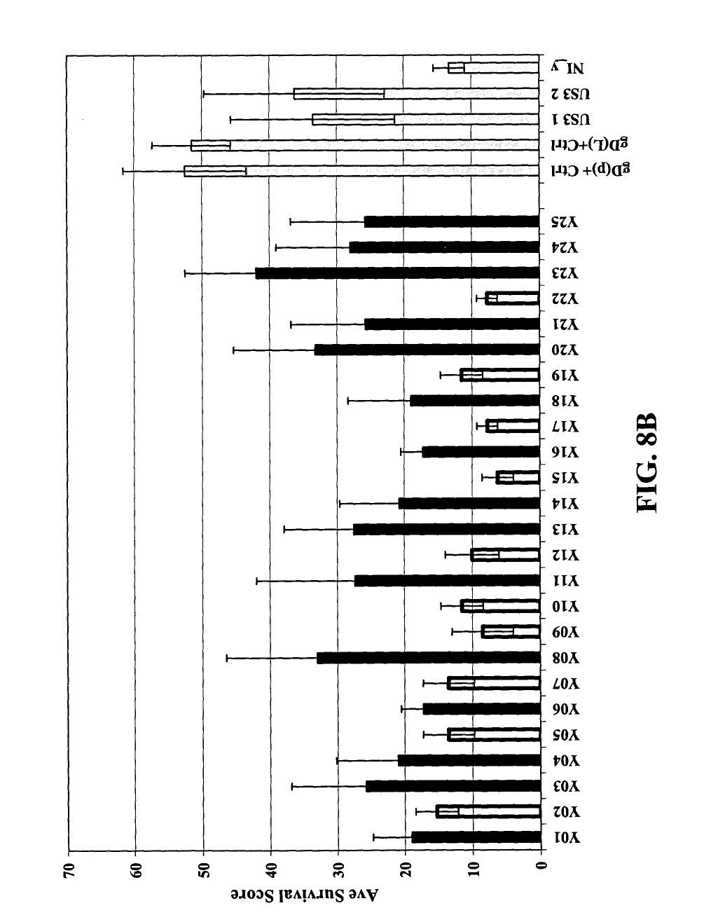

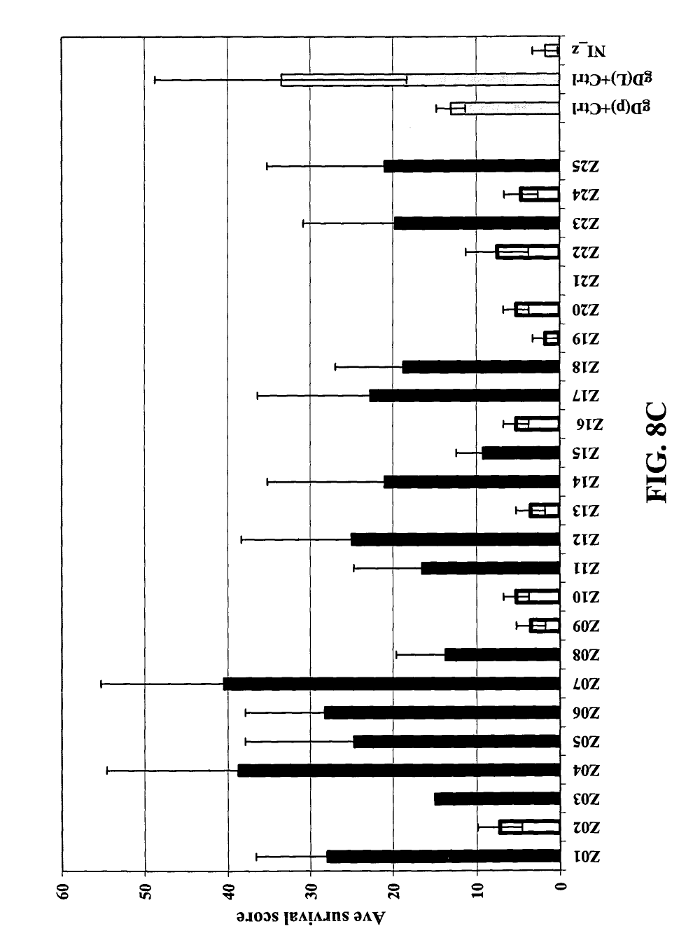

The challenge assay results of the mice immunized with the X, Y, and Z sets of matrix-arrayed library-inocula are depicted in FIG. 7 and FIG. 8. In FIG. 7, the raw survival rates are provided for days 7 through 10, and the endpoint day (last day monitored before sacrifice). In FIG. 8 survival scores are plotted. These scores were derived in order to compare levels of protection between the sets of X, Y, and Z groups. Animal survival data recorded for days 6 through day 12 were used to determine the survival score for each of the 75 study and control groups. An individual animal score was calculated by summing the day-numbers post-exposure (days 6 through 12) for which the animal lived. An average score and standard error was calculated for each group of mice and used for graphing the group results.

### Example 13

### Matrix Analyses of Protection Data

In order to analyze the results with respect to a three-dimensional matrix, the average group-survival scores were normalized to that of the positive control group commonly included in each of the X, Y, and Z data sets. The purpose of normalization to a standard (gD control) is to minimize the impact of any unintended differences between the three independently conducted X, Y, and Z challenge studies. A normalized group score of “0” indicates that no mice were alive beyond day 6 post-infection; a group score of “1.0” indicates that the group's survival score was equivalent to that of the positive control mice tested in parallel, which were immunized with a full 250 ng dose of the protective antigen gD. The average normalized survival score of the three groups (X, Y, and Z) of negative control mice was calculated to be 0.166.

These results of the challenge-protection assays of the 75 study groups were subjected to matrix analyses that permitted protective candidates to be inferred by either i) triangulation or ii) quantitative ranking. For the triangulation method, the survival scores were used to categorize each test group as either positive or negative. An average of 15 of the 25 test groups from each of the three data sets showed group survival scores above the negative controls. Consequently, the top-scoring 15 groups were designated as positives for equilateral matrix analysis, and the ORF-pools used to inoculate these animal groups were pursued. The planar intersections of the positive pools indicated 3,375 loci within the virtual cube that was originally used to design these pools. Since only 127 ORFs were arrayed in a grid with 15,625 possible positions (25×25×25), most loci were not filled, enabling triangulation to pinpoint 23 ORF-containing intersections. The ORFs located at these cross-hairs are resident in each of one positively scoring X, Y, and Z pool, and thereby they were candidates for causing the observed mouse protection. Thus cross-hair triangulation and low occupancy enabled 104 of 127 ORFs to be culled, an 82% reduction of the library. The 23 ORFs, corresponding to 21 different HSV-1 genes including gD, are listed in Table 5. The nucleotide length of the library-tested ORF, the size of the derivative gene, and the grid coordinates of the ORF are provided. Since 15 groups had been chosen from each axis to analyze, it was estimated that approximately 15 ORFs are responsible for the observed protection. Fewer than 15 ORFs may be true candidates if one or more groups were mis-categorized as positive, or if one or more ORF is pooled with another ORF that masked the protective activity. Even though the inventors were testing each ORF in three independent pools of other ORFs, identification by triangulation analysis requires a cross-hair, or positive scores in all three of an ORF's resident pools.

TABLE 5
Intersection Analysis By Triangulation

| | Fragment Size | Gene Size | |
|---|---|---|---|
| ORF name | (bp) | (bp) | Resident pools |
| RL1_a_a | 339 | 747 | X20, Y1, Z15 |
| UL1_a | 588 | 675 | X20, Y1, Z8 |
| UL11_a | 249 | 291 | X23, Y6, Z12 |
| UL13_b | 801 | 1557 | X09, Y20, Z15 |
| UL15_a_a | 309 | 2208 | X21, Y16, Z18 |
| UL16_a_c | 309 | 1122 | X6, Y24, Z4 |
| UL17_a | 984 | 2112 | X1, Y14, Z4 |
| UL17_b | 1053 | 2112 | X11, Y3, Z6 |
| UL18_a | 939 | 957 | X22, Y23, Z3 |
| UL21_b | 795 | 1608 | X22, Y13, Z3 |
| UL25_a | 831 | 1743 | X16, Y20, Z1 |
| UL28_a | 1065 | 2358 | X6, Y25, Z18 |
| UL36_b | 1320 | 9495 | X17, Y6, Z3 |
| UL37_b | 1128 | 3372 | X09, Y11, Z12 |
| UL41_a | 1401 | 1470 | X10, Y13, Z04 |
| UL43_a | 1182 | 1305 | X21, Y3, Z17 |
| UL44_a | 708 | 1536 | X12, Y16, Z5 |
| UL5_a | 1290 | 2649 | X12, Y4, Z1 |
| UL52_c | 1020 | 3177 | X25, Y25, Z15 |
| UL54_a | 702 | 1539 | X09, Y16, Z05 |
| UL54_b | 711 | 1539 | X23, Y13, Z23 |
| US5_a | 261 | 279 | X10, Y24, Z12 |
| US6_a | 1089 | 1185 | X16, Y20, Z6 |

Although one of the advantages of the triangulation method is that any pinpointed candidate has been tested in triplicate, the requirement for three positive readouts can also be a disadvantage. In addition it does not enable the inferred protective capacity of one ORF relative to one another in the grid to be discerned. In a second matrix analysis a quantitative ranking was performed that addresses both of these potential pitfalls. The ranking method accommodates for the possibility that a protective ORF may reside in a pool carrying a negative ORF. If the other two resident pools score well, the protective ORF can still be identified based on a favorable three-pool cumulative score. Quantitation also allows the assignment of a score value to each ORF, and thereby derive a rank-sorted list of all the constituent ORFs in the entire genomic grid.

For the ranking method, each ORF was given a score-value that is based on individual scores of the three groups that had been inoculated with the three pools (one X, one Y, and one Z) containing any particular ORF. The normalized scores of the three X, Y, and Z “coordinates” of every ORF in the grid were summed, averaged, and standard errors were calculated. Table 6 displays a rank-sorted list of ORFs based on average survival scores of their resident pools. ORF fragment length, derivative gene size, and each ORFs grid coordinates are also provided.

TABLE 6
Intersection Analysis By Quantitative Ranking, Survival Score

| | | Fragment Size | | |
|---|---|---|---|---|
| ORF name | Rank | (bp) | GeneSize (bp) | Resident pools |
| UL16_a_c | 1 | 309 | 1122 | X6, Y24, Z4 |
| UL8_a | 2 | 1039 | 2253 | X17, Y21, Z19 |
| UL18_a | 3 | 939 | 957 | X22, Y23, Z3 |
| UL43_a | 4 | 1182 | 1305 | X21, Y3, Z17 |
| UL17_a | 5 | 984 | 2112 | X1, Y14, Z4 |
| UL21_b | 6 | 795 | 1608 | X22, Y13, Z3 |
| UL52_b_a | 7 | 315 | 3177 | X16, Y12, Z07 |
| UL30_c | 8 | 1249 | 3708 | X08, Y08, Z07 |
| UL41_a | 9 | 1401 | 1470 | X10, Y13, Z04 |
| US6_a | 10 | 1089 | 1185 | X16, Y20, Z6 |
| UL6_b | 11 | 946 | 2031 | X21, Y10, Z17 |
| UL25_a | 12 | 831 | 1743 | X16, Y20, Z1 |
| UL28_b_b | 13 | 312 | 2358 | X04, Y07, Z12 |
| UL15_a_a | 14 | 309 | 2208 | X21, Y16, Z18 |
| UL40_a | 15 | 904 | 1023 | X06, Y09, Z06 |
| RS1_a | 16 | 1273 | 3897 | X22, Y12, Z11 |
| UL47_b | 17 | 973 | 2082 | X22, Y20, Z16 |
| UL26_a | 18 | 877 | 1908 | X12, Y17, Z04 |
| UL37_c | 19 | 1083 | 3372 | X4, Y5, Z23 |
| UL28_a | 20 | 1065 | 2358 | X6, Y25, Z18 |
| UL26.5_a | 21 | 973 | 990 | X21, Y19, Z11 |
| UL49A_a | 22 | 166 | 276 | X24, Y06, Z07 |
| UL17_b | 23 | 1053 | 2112 | X11, Y3, Z6 |
| UL33_a | 24 | 325 | 393 | X08, Y19, Z07 |
| US4_a | 25 | 661 | 717 | X21, Y23, Z21 |
| UL36_d_c | 26 | 426 | 9495 | X19, Y23, Z06 |
| UL5_a | 27 | 1290 | 2649 | X12, Y4, Z1 |
| UL36_g_c | 28 | 426 | 9495 | X22, Y21, Z09 |
| UL55_a | 29 | 478 | 561 | X03, Y02, Z04 |
| UL37_b | 30 | 1128 | 3372 | X09, Y11, Z12 |
| UL13_a | 31 | 799 | 1557 | X04, Y13, Z02 |
| UL29_b | 32 | 1141 | 3591 | X18, Y11, Z06 |
| UL8_b | 33 | 1087 | 2253 | X13, Y08, Z14 |
| US5_a | 34 | 261 | 279 | X10, Y24, Z12 |

The ORFs were also rank-sorted based on the p-value calculated by student's t test of the difference between an ORF's survival scores and that of the negative controls. Table 7 enumerates the 34 ORFs displaying p-values of ≦0.05. ORF fragment length, derivative gene size, and each ORF's grid coordinates are also provided. Because 34 ORFs were determined to be above the p-value cut-off used in Table 7, the inventors chose also to arbitrarily list the top 34 ORFs by survival score in Table 6.

TABLE 7
Intersection Analysis By Quantitative Ranking, Ttest

| | | Fragment Size | Gene Size | |
|---|---|---|---|---|
| ORF name | Rank | (bp) | (bp) | Resident pools |
| UL54_b | 1 | 711 | 1539 | X23, Y13, Z23 |
| UL1_a | 2 | 588 | 675 | X20, Y1, Z8 |
| UL28_a | 3 | 1065 | 2358 | X6, Y25, Z18 |
| RL1_a_a | 4 | 339 | 747 | X20, Y1, Z15 |
| RL2_a_a | 5 | 345 | 2328 | X10, Y14, Z02 |
| UL13_b | 6 | 801 | 1557 | X09, Y20, Z15 |
| UL25_a | 7 | 831 | 1743 | X16, Y20, Z1 |
| US8A_a | 8 | 433 | 480 | X12, Y11, Z02 |
| US6_a | 9 | 1089 | 1185 | X16, Y20, Z6 |
| UL8_b | 10 | 1087 | 2253 | X13, Y08, Z14 |
| UL36_b | 11 | 1320 | 9495 | X17, Y6, Z3 |
| UL18_a | 12 | 939 | 957 | X22, Y23, Z3 |
| UL36_a | 13 | 1353 | 9495 | X11, Y18, Z22 |
| UL43_a | 14 | 1182 | 1305 | X21, Y3, Z17 |
| UL16_a_c | 15 | 309 | 1122 | X6, Y24, Z4 |
| UL31_a | 16 | 907 | 921 | X13, Y21, Z17 |
| UL52_a | 17 | 1018 | 3177 | X11, Y07, Z03 |
| UL52_c | 18 | 1020 | 3177 | X25, Y25, Z15 |
| UL37_b | 19 | 1128 | 3372 | X09, Y11, Z12 |
| UL21_b | 20 | 795 | 1608 | X22, Y13, Z3 |
| UL17_b | 21 | 1053 | 2112 | X11, Y3, Z6 |
| UL49_a | 22 | 841 | 906 | X14, Y01, Z20 |
| UL44_b | 23 | 751 | 1536 | X23, Y02, Z02 |
| UL22_b | 24 | 1186 | 2517 | X10, Y14, Z16 |
| UL51_a | 25 | 685 | 735 | X02, Y14, Z10 |
| UL28_b_b | 26 | 312 | 2358 | X04, Y07, Z12 |
| UL15_a_a | 27 | 309 | 2208 | X21, Y16, Z18 |
| UL36_f_b | 28 | 420 | 9495 | X13, Y06, Z23 |
| UL16_a_b | 29 | 354 | 1122 | X23, Y10, Z03 |
| UL37_c | 30 | 1083 | 3372 | X4, Y5, Z23 |
| US5_a | 31 | 261 | 279 | X10, Y24, Z12 |
| UL39_b | 32 | 1093 | 3414 | X01, Y10, Z18 |
| UL20_a | 33 | 628 | 669 | X11, Y07, Z02 |
| UL11_a | 34 | 249 | 291 | X23, Y6, Z12 |

The inventors have found that the cross-hair triangulating and quantitative ranking methods predominantly identify the same ORFs. In particular, all 23 ORFs identified by triangulation were also identified by ranking. However the two quantitative analyses enabled more ORFs to be identified with inferred protective capacities. The most useful distinction between the two analysis approaches is that the cumulative scoring enables all of the herpesvirus coding sequences to be ranked by inferred utility. Table 8 lists the ORFs inferred, based on the preceding analyses of the DELI data, to be candidate vaccines. ORFs identified by at least two of the three analyses are listed as “repeated hits” and the SEQ IDs correspond to these ORFs.

TABLE 8
Condensed Output From the DELI Screen Analyses

| | | SEQ ID No. for |
|---|---|---|
| All ORFs | Repeated ORFs | Repeated ORFs |
| RL1_a_a | RL1_a_a | SEQ ID NO: 1 |
| RL2_a_a | UL1_a | SEQ ID NO: 5 |
| RS1_a | UL5_a | SEQ ID NO: 9 |
| UL1_a | UL8_b | SEQ ID NO: 13 |
| UL5_a | UL11_a | SEQ ID NO: 17 |
| UL6_b | UL13_b | SEQ ID NO: 21 |
| UL8_a | UL15_a_a | SEQ ID NO: 25 |
| UL8_b | UL16_a_c | SEQ ID NO: 29 |
| UL11_a | UL17_a | SEQ ID NO: 35 |
| UL13_a | UL17_b | SEQ ID NO: 37 |
| UL13_b | UL18_a | SEQ ID NO: 41 |
| UL15_a_a | UL21_b | SEQ ID NO: 45 |
| UL16_a_b | UL25_a | SEQ ID NO: 49 |
| UL16_a_c | UL28_a | SEQ ID NO: 59 |
| UL17_a | UL28_b_b | SEQ ID NO: 61 |
| UL17_b | UL36_b | SEQ ID NO: 69 |
| UL18_a | UL37_b | SEQ ID NO: 73 |
| UL20_a | UL37_c | SEQ ID NO: 75 |
| UL21_b | UL41_a | SEQ ID NO: 79 |
| UL22_b | UL43_a | SEQ ID NO: 83 |
| UL25_a | UL44_a | SEQ ID NO: 87 |
| UL26.5_a | UL49_a | SEQ ID NO: 91 |
| UL26_a | UL52_c | SEQ ID NO: 95 |
| UL28_a | UL54_b | SEQ ID NO: 99 |
| UL28_b_b | US5_a | SEQ ID NO: 107 |
| UL29_b | US6_a | SEQ ID NO: 113 |
| UL30_c | | |
| UL31_a | | |
| UL33_a | | |
| UL36_a | | |
| UL36_b | | |
| UL36_d_c | | |
| UL36_f_b | | |
| UL36_g_c | | |
| UL37_b | | |
| UL37_c | | |
| UL39_b | | |
| UL40_a | | |
| UL41_a | | |
| UL43_a | | |
| UL44_a | | |
| UL47_b | | |
| UL49_a | | |
| UL51_a | | |
| UL52_a | | |
| UL52_b_a | | |
| UL52_c | | |
| UL54_a | | |
| UL54_b | | |
| UL55_a | | |
| US4_a | | |
| US5_a | | |
| US6_a | | |
| US8A_a | | |

In Table 9 the derivative genes of the ORFs identified by the three analyses of the DELI data are listed and compared with the results of the RELI screen of randomly-generated HSV-1 gene fragments. The final column provides a list of the 23 genes, corresponding to 26 ORF hits repeatly indicated by the ELI analyses.

TABLE 9
Summary Of Genes Identified By Analyses Of
The HSV-1 DELI And RELI Screens.

| | | Ranking | | | | |
|---|---|---|---|---|---|---|
| | | DELI, | DELI, | | Summary | |
| Triangulation | | by | by | | Repeat | SEQ ID NOs. |
| RELI | DELI | Score | TTest | rank | Genes | For Repeat Genes |
| UL17 | RL1 | UL16 | UL54 | 1 | RL1 | SEQ ID NO: 3 |
| UL24 | UL1 | UL8 | UL1 | 2 | UL1 | SEQ ID NO: 7 |
| UL27 | UL5 | UL18 | UL28 | 3 | UL5 | SEQ ID NO: 11 |
| UL28 | UL11 | UL43 | RL1 | 4 | UL8 | SEQ ID NO: 15 |
| UL29 | UL13 | UL17 | RL2 | 5 | UL11 | SEQ ID NO: 19 |
| UL36 | UL15 | UL21 | UL13 | 6 | UL13 | SEQ ID NO: 23 |
| UL50 | UL16 | UL52 | UL25 | 7 | UL15 | SEQ ID NO: 27 |
| US3 | UL17 | UL30 | US8 | 8 | UL16 | SEQ ID NO: 31 |
| US6 | UL18 | UL41 | US6 | 9 | UL17 | SEQ ID NO: 39 |
| US8 | UL21 | US6 | UL8 | 10 | UL18 | SEQ ID NO: 43 |
| | UL25 | UL6 | UL36 | 11 | UL21 | SEQ ID NO: 47 |
| | UL28 | UL25 | UL18 | 12 | UL25 | SEQ ID NO: 51 |
| | UL36 | UL28 | UL43 | 13 | UL28 | SEQ ID NO: 63 |
| | UL37 | UL15 | UL16 | 14 | UL36 | SEQ ID NO: 71 |
| | UL41 | UL40 | UL31 | 15 | UL37 | SEQ ID NO: 77 |
| | UL43 | RS1 | UL52 | 16 | UL41 | SEQ ID NO: 81 |
| | UL44 | UL47 | UL37 | 17 | UL43 | SEQ ID NO: 85 |
| | UL52 | UL26 | UL21 | 18 | UL44 | SEQ ID NO: 89 |
| | UL54 | UL37 | UL17 | 19 | UL49 | SEQ ID NO: 93 |
| | US5 | UL26.5 | UL49 | 20 | UL52 | SEQ ID NO: 97 |
| | US6 | UL49 | UL44 | 21 | UL54 | SEQ ID NO: 101 |
| | | UL33 | UL22 | 22 | US5 | SEQ ID NO: 109 |
| | | US4 | UL51 | 23 | US6 | SEQ ID NO: 115 |
| | | UL36 | UL15 | 24 | | |
| | | UL5 | US5 | 25 | | |
| | | UL55 | UL39 | 26 | | |
| | | UL13 | UL20 | 27 | | |
| | | UL29 | UL11 | 28 | | |
| | | US5 | | 29 | | |

An ELI protection study might also have been analyzed without matrix arraying. If the 127 ORFs had been partitioned into pools of 5 ORFs as above, and 15 positive groups were selected as above, then only 40% ((10 negative groups)×(5 ORFs/group))/127) of the unprotective ORFs would have been culled. Each ORF would have been tested only once, in only one ORF mixture.

### Example 14

### Analysis of DELI-Identified ORFS

In a directed LEE library screen, 23 HSV-1 ORFs were identified as vaccine candidates by triangulation and another 31 were identified by either/both quantitative scoring and p-value sorting. Among these ORFs is glycoprotein D (gD), a previously studied HSV vaccine candidate that has generated variable results in clinical trials. The gene encoding gD, US6, was identified by all three of our DELI analyses. The second HSV antigen most studied as a possible vaccine component is glycoprotein B (gB). Its absence in our list of ORF candidates can be explained by comparing the ORF design to the known. B-cell determinants of gB. The gene-splitting program for primer design breaks genes greater than 1,500 bp into subgenes, and in particular the 2,715 bp gB gene was arbitrarily divided into two subgene ORFs. ORF “a” ends at amino acid (aa) 461, and ORF “b” starts at aa 444. A prominent H-2d (i.e. BALB/c mice) domain detected by a known neutralizing antibody to HSV-1 spans amino acids 290 to 520 (Navarro et al., 1992). In the RELI screen of the HSV-1 genome, using populations of randomly fragmented ORFs, fragments of both gB and gD were identified as candidate protective ORFs, along with 8 other ORFs. The genes corresponding to 4 of the 8 novel candidates identified by RELI were also identified in the DELI screen (US8, UL17, UL28, and UL29).

Among the novel candidates, there is also some overlapping results between thr RELI and DELI screens. For example, 5 different ORFs encoding different portions of the very large tegument protein UL36 were inferred to hold some level of protective capacity in the DELI screen. A DNA fragment triangulated with the RELI results encodes a portion of UL36 (aa 338 to 509) that spans 2 of these 5 DELI hits (aa 1 to 461; aa 444 to 897). In another case, both portions of UL17, which was split into 2 ORFs for DELI, were identified in the DELI screen, and a random UL17 fragment was identified by RELI. Likewise, both fragments of the full UL28 gene were identified by DELI, and a random fragment of it was identified by RELI. The remaining ORFs inferred to carry some protective capacity by this screen correspond to a varied set of cytoplasmic, nuclear, and structural genes. The genes indicated by at least two of the three analyses of the DELI screen are listed in Table 10 with the viral products and/or the biological processes that these gene products are known or suggested to be involved in are provided. Categories of gene products multiply hit include DNA packaging, tegument, capsid and immediate early proteins, glycoproteins and components of the helicase-primase complex. A virulence factor, DNAse, metabolic protein, and a few products without know functions are also indicated as candidates.

TABLE 10
Name Of HSV-1 Gene Product And/Or Its
Known Or Proposed Biological Activity

| ORF | Gene product /activity |
|---|---|
| RL1 | ICP34.5, Neurovirulence factor, |
| | Inhibition of host protein synthesis |
| UL1 | Glycoprotein L Viral spread |
| UL5 | Viral genome replication, DNA helicase-primase subunit |
| UL8 | Intracellular protein transport |
| UL11 | Myristylated tegument protein |
| | Viral capsid envelopment |
| UL13 | Induction of apoptosis by virus, |
| | ATP-binding, protein kinase |
| UL15 | Viral DNA packaging protein |
| UL16 | DNA packaging, capsid maturation protein |
| UL17 | Viral DNA cleavage and packaging |
| UL18 | Capsid protein |
| UL21 | Cytoskeleton organization and biogenesis |
| UL25 | Capsid-associated tegument, viral assembly protein |
| UL28 | ICP18.5 |
| | Viral DNA packaging protein |
| UL36 | ICP1-2, |
| | Very large tegument protein Viral egress |
| UL37 | Viral budding |
| UL41 | Vhs |
| | Host defense evasion, Inhibition of cytokine production |
| UL43 | Tegument protein |
| UL44 | Glycoprotein C |
| | Enhancement of virulence |
| UL49 | VP22 |
| | Cell to cell viral spread |
| UL52 | DNA helicase-primase subunit |
| | Initiator for ATG codons |
| UL54 | ICP27 |
| | Perturbation of host cell transcription |
| US5 | GJ |
| | viral inhibition of apoptosis |
| US6 | Glycoprotein D |
| | Viral induced cell-cell fusion |

Table 11 presents the nucleotide similarities and identities of the gene products encoded by the HSV-1. ORFs identified in the ELI screen to homologs in other herpesviruses. These sequence comparisons may indicate that the HSV-1 homologs could carry protective capacities. For example the gD gene product of BHV has been shown to be protective against BHV, as is its glycoprotein homologue from HSV-1 and HSV-2. Notably, a number of DELI HSV-1 hits show similarities to other herpesvirus gene products that are significantly higher than that of gD. It also suggests that vaccination with genes from one virus might heterologously protect against exposure to a different herpesvirus.

TABLE 11
Examples Of Percent Amino Acid Identities/Similarities
To Herpesvirus Homologs.

| ORF | HSV2 | VZV | BHV | EHV | CMV | CHV |
|---|---|---|---|---|---|---|
| RL1aa | 41/47 | 32/39 | 29/33 | 29/33 | 24/26 | 26/31 |
| UL1a | 70/80 | 29/50 | 28/33 | 31/47 | 26/37 | 58/66 |
| UL5a | 90/92 | 62/78 | 64/77 | 67/81 | 32/51 | 85/92 |
| UL8b | 78/83 | 26/42 | 31/43 | 29/46 | 43/46 | 52/62 |
| UL11a | 73/80 | 34/54 | 35/45/ | 35/52 | 26/35 | 59/70 |
| UL13b | 80/88 | 33/54 | 34/44 | 34/54 | 28/41 | 58/70 |
| UL15aa | 96/98 | 44/67 | 55/65 | 56/70 | 34/46 | 80/87 |
| UL16ac | 72/79 | 34/50 | 42/58 | 42/49 | 24/34 | 63/73 |
| UL17a | 76/83 | 35/50 | 35/44 | 36/50 | 24/32 | 36/48 |
| UL17b | 87/90 | 33/50 | 39/50 | 38/55 | 33/48 | 74/81 |
| UL18a | 92/95 | 42/61 | 47/63 | 43/65 | 28/42 | 83/90 |
| UL21b | 82/88 | — | 34/40 | 27/43 | 33/46 | 56/70 |
| UL25a | 85/88 | 42/58 | 49/60 | 46/63 | 29/38 | 71/80 |
| UL28a | 88/90 | 43/56 | 49/61 | 47/60 | 23/44 | 83/88 |
| UL28bb | 99/100 | 63/78 | 68/81 | 67/85 | 31/55 | 93/96 |
| UL36b | 80/87 | 32/47 | 31/42 | 30/47 | 27/40 | 61/75 |
| UL37b | 90/95 | 32/46 | 28/43 | 31/50 | 28/50 | 76/83 |
| UL37c | 80/85 | 25/42 | 27/41 | 23/41 | 31/44 | 66/77 |
| UL41a | 85/88 | 39/56 | 32/48 | 33/51 | 34/44 | 70/80 |
| UL43a | 65/71 | 28/35 | 24/30 | 31/35 | 25/33 | 44/52 |
| UL44a | 54/61 | 28/40 | 23/40 | 26/37 | 24/35 | 37/46 |
| UL49a | 68/75 | 25/32 | 26/33 | 32/42 | 25/35 | 44/55 |
| UL52c | 85/89 | 48/65 | 48/63 | 42/59 | 31/44 | 71/80 |
| UL54b | 91/94 | 40/61 | 46/60 | 43/62 | 26/42 | 70/82 |
| US5a | 48/62 | 39/51 | 27/30 | 33/39 | 29/35 | 27/41 |
| US6a | 83/89 | 25/44 | 27/38 | 27/42 | 29/41 | 61/74 |

In this study, two different promoter-leader fusions were linked to each of the tested ORF. Since these LEE constructs were co-delivered it cannot be discern whether the secretory or proteasome targeting led to a more protective response. However, the inventors previously have found that simultaneous delivery of ORFs did not interfere with any individual ORF-generated response.

### Example 15

### Comparison of Directed-LEE Library Screening

### to the Random ELI Screening Methodology

In the random ELI (RELI) screening protocol 10 ORFs including fragments of the gB and gD genes from the HSV-1 genome were inferred by matrix triangulation to be candidates for protective antigens. Triangulation of the DELI data revealed 23 ORFs with inferred protective utility. A number of genes in these two output groups overlapped, while others were unique. Table 12 delineates some technical parameters that are likely to have influenced the outcomes of the two ELI studies.

The results of the two protection studies reflect both these differences and similarities in design. Among the 10 gene fragments identified as protective candidates in the RELI grid, 6 of the derivative genes were also on the list of top 23 genes identified in the DELI protection screen. Among the 6 RELI gene fragments that tested positive when tested individually, all but two of the derivative genes were also identified in the DELI grid. These two outliers were gB and US3. Glycoprotein B (UL27) was identified only in the RELI screen most likely for technical reasons, as described above. Likewise US3 was only identified in the RELI grid, most likely for technical reasons also. In particular, a fragment of US3 was functionally-selected from a population of random subgenes in the RELI study. However in the DELI study, the full-length US3 gene was tested. Recent studies have demonstrated that constructs carrying the full-length sequence are not protective.

TABLE 12
Two ELI Screens Compared.

| RELI | DELI |
|---|---|
| Statistically-assumed coverage of | Complete, defined coverage |
| genome | |
| Plasmid GMCSF was included | No adjuvant |
| in round 1 | |
| Any particular ORF is tested unknown | Each gene tested in triplicate |
| number of times | |
| Pools sizes in round 1 of ˜600 | Pools sizes of 5 ORFs |
| ORFs expressed in plasmids, with | ORFs generated in vitro for LEE |
| potential for cloning biases and | expression |
| contamination | |
| Each ORF fused to sequences encoding | Each ORF fused to both LS |
| either tPA or UB targeting peptides | and UB sequences for |
| | intracellular targeting |
| Library comprised of random ˜800 bp | Library comprised of |
| physically-generated genomic | sequence-defined 1500 bp ORFs |
| fragments | |
| ORFs delivered biolistically into ears | ORFs delivered biolistically |
| and by injection into leg muscles | into ears |
| Hairless mice used in round 1 then | BALB/c mice only |
| BALB/c for subsequent rounds | |

### Example 16

### Testing of Individual DELI ORFS as Vaccine

### Candidates

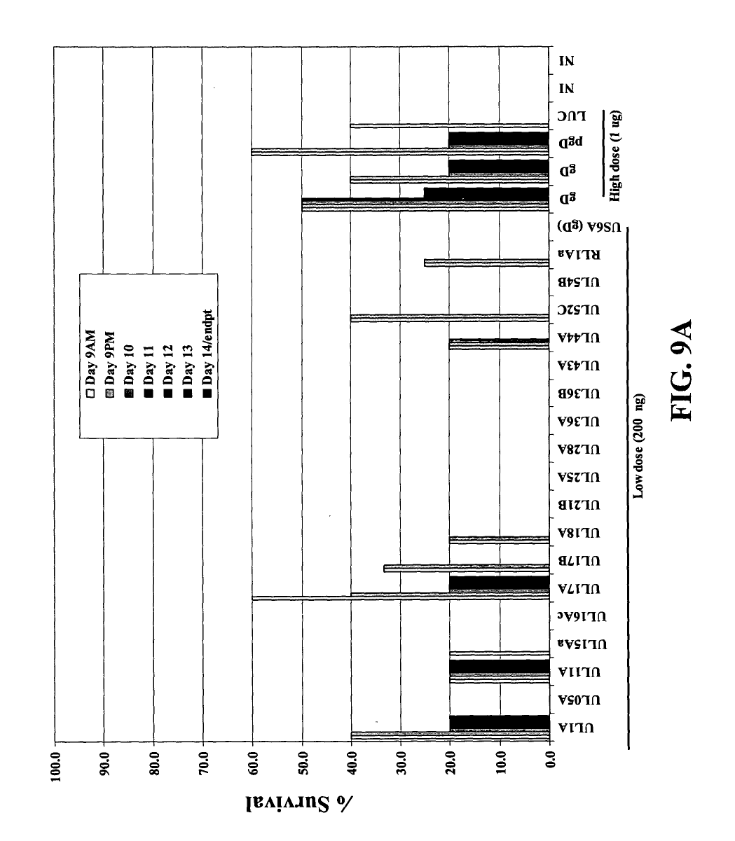

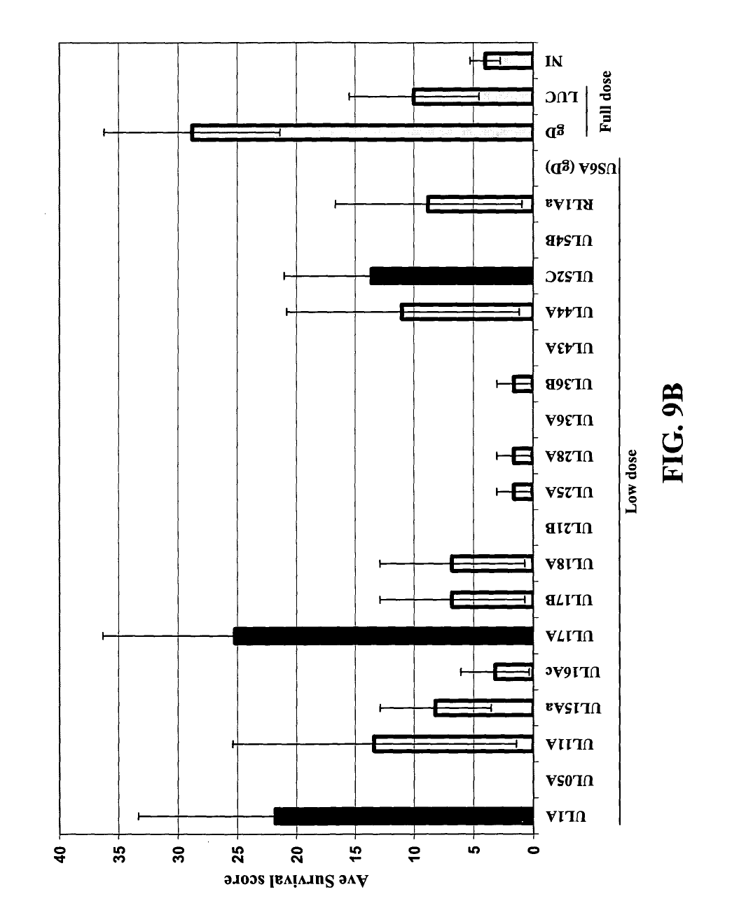

From the qualitative triangulation analysis of the challenge survival assay results, 26 HSV-1 ORFs (from 23 genes) were inferred to carry protective capacities. From this set, 19 ORFs were PCR-amplified and prepared again as LEEs on gold microprojectiles. These antigens were then gene-gun delivered as single genes (200 ng) into groups of 5 BALB/c mice. Each inoculum also contained 800 ng of empty vector DNA, used to facilitate microprojectile preparation. Boosts were administered at weeks 4 and 8, followed by virus exposure at week 11. These mice were lethally challenged with HSV-1 using a scarification route as performed earlier and then survival was monitored twice daily for 14 days. Nine groups of mice survived longer than the positive control group which was administered gD (US6) at the same dose as the test genes. This gD group survived until day 8; those ORFs associated with longer survival are: UL1a, UL11a, UL15a, UL17a, UL18a, UL44a, UL52c, and RL1a. At the completion of the study (14-day endpoint) groups of mice immunized with UL1a, UL11a, and UL17a still maintained a survivor. Other control groups were immunized with a full 1 ug dose of gD, constructed in both an LEE and as a plasmid, and a non-HSV-1 gene, LUC carried in the CpG rich plasmid pCMVi. The survival rates at several days through the monitoring period are plotted in FIG. 9A. Survival scores were calculated for the period from day 8 through 12, and these are graphed in FIG. 9B. Calculating a single survival score for each mouse that integrates the multiple data points through the monitoring period enables group averages and standard errors to be determined. Analysis indicates that immunization with UL1a, UL17a, and UL52c generates survival scores that are non-overlapping with the non-immunized control group. The remaining ORFs from the triangulation and quantitative analyses will be next tested individually.

### Example 17

### Creation and Testing of Vaccines using

### Combinations of the ELI-Identified Herpesvirus Nucleic Acid

### and Amino Acid Sequences

The Herpesvirus sequences and antigens showing protection may be developed into vaccines for Herpesvirus in humans and animals in the following manner. The genetic-antigens, genetic-antigen fragments, protein antigens or protein antigen fragments may be combined with one another, including the previously identified glycoproteins B and D antigens to produce an improved vaccine. These may be delivered by a combination of modalities, such as genetic, protein, or live-vectors. Alternatively, the functional or sequence homologs of the identified antigen candidates from multiple herpesviruses might be combined to produce broader protection against multiple species in one vaccine.

### Example 18

### Creation and Testing of Vaccines Against Other

### Herpesviruses using the Identified Herpesvirus Nucleic Acid

### and Amino Acid Sequences

The Herpesvirus sequences and antigens disclosed in this application are envisioned to be used in vaccines for Herpesvirus in humans and commercially important animals. However, these Herpesvirus sequences may be used to create vaccines for other viral species as well. For example, one may use the information gained concerning Herpesvirus to identify a sequence in another viral pathogen that has substantial homology to the Herpesvirus sequences. In many cases, this homology would be expected to be more than a 30% amino acid sequence identity or similarity and could be for only part of a protein, e.g., 30 amino acids, in the other species. The gene encoding such identity/similarity may be isolated and tested as a vaccine candidate in the appropriate model system either as a protein or nucleic acid. Alternatively, the Herpesvirus homologs may be tested directly in an animal species of interest. Given there are a limited number of genes to screen, and that the genes have been demonstrated to be protective in another species the probability of success should be high. Alternatively, proteins or peptides corresponding to the homologs to the Herpesvirus genes may be used to assay in animals or humans for immune responses in people or animals infected with the relevant pathogen. If such immune responses are detected, particularly if they correlated with protection, then the genes, proteins or peptides corresponding to the homologs may be tested directly in animals or humans as vaccines.

### Example 19

### Creation and Testing of Commercial Vaccines

### using Herpesvirus Nucleic Acid and Amino Acid Sequences

The vaccine candidates described herein may be developed into commercial vaccines. For example, the genes identified may be converted to optimized mammalian expression sequences by altering the codons to correspond with a codon preference of an animal to be vaccinated. This is a straightforward procedure, which can be easily done by one of skill in the art. Alternatively, a protective gene vaccine might be sequence-optimized by shuffling homologs from other herpesviruses (Stemmer et al., 1995). This might increase efficacy against HSV-1 exposure and/or provide a vaccine that protects against multiple herpesviruses. The genes may then be tested in the relevant host, for example, humans, for protection against infection. Genetic immunization affords a simple method to test vaccine candidate for efficacy and this form of delivery has been used in a wide variety of animals including humans. Alternatively, the genes may be transferred to another vector, for example, a vaccinia vector, to be tested in a relevant host.

Alternatively, the corresponding protein, with or without adjuvants may also be tested. These tests may be done on a relatively small number of animals. Once conducted, a decision can be made as to how many of the protective antigens to include in a larger test. Only a subset may be chosen based on the economics of production. A large field trial may be conducted using a preferred formulation. Based on the results of the field trial, possibly done more than once at different locations, a commercial vaccine may then be produced.

### Example 20

### Creation and Testing of Vaccines Against Other

### Pathogens using Herpesvirus Nucleic Acid and Amino Acid

### Sequences

Since HSV-1 has a similar biology to other herpesviruses, the inventors take advantage of the screening already accomplished on the HSV-1 genome to test other herpesviruses for homologs corresponding to the ones from HSV-1 as vaccine candidates. Those of ordinary skill may expect that, as one moved evolutionarily away from HSV-1, the likelihood that the homologs would protect would presumably decline. Once the homologs have been identified and isolated, they may be tested in the appropriate animal model system for efficacy as a vaccine. For example, other herpesvirus homologs, genes or proteins, may be tested in a mouse herpesvirus model.

One of ordinary skill has access to herpesvirus sequences disclosed in this specification, or to additional sequences determined to be protective using any of the methods disclosed in this specification, a computer-based search of relevant genetic databases may be run in order to determine homologous sequences in other pathogens. For example, these searches can be run in the BLAST database in GenBank.

Once a sequence which is homologous to a protective sequence is determined, it is possible to obtain the homologous sequence using any of a number of methods known to those of skill. For example, PCR amplification of a homologous gene(s) from a pathogen from genomic DNA and place the genes in an appropriate genetic immunization vector, such as a plasmid or LEE. These homologous genes may then be tested in an animal model appropriate for the pathogen for which protection is sought, to determine whether homologs of herpesvirus genes will protect a host from challenge with that pathogen.

It is contemplated that the herpesvirus genes that are disclosed herein as protective, or determined to be protective using the methods disclosed herein, to obtain protective sequences from a first non-herpesvirus organism, then to use the protective sequences from the non-herpesvirus organism to search for homologous sequences in a second non-herpesvirus or herpesvirus organism. So long as a protective herpesvirus sequence is used as the starting point for determining at least one homology in such a chain of searches and testing, such methods are within the scope of this invention.

### Example 21

### Creation and Testing of Therapeutic Vaccines

### using Herpesvirus Nucleic Acid and Amino Acid Sequences

The vaccine candidates described herein may be useful not only prophylactically but also therapeutically. For example, reactivation of latent herpes infections is a significant health issue (Keadle et al., 1997; Nesburn et al., 1998; Nesburn et al., 1994; Nesburn et al., 1998). Vaccine candidates identified in this prophylactic screen are envisioned to be used to immunize HSV infected subjects to eliminate infection or to ameliorate disease symptoms associated with subsequent activation of herpesvirus proliferation.

Once a subject or patient has been identified as having a herpesvirus infection the vaccination methods and compositions of the invention may be used as a therapy. Methods are known for optimizing the amount, schedule and route of administration, when taken in light of the present specification.

### Example 22

### Creation and Testing of Therapeutic Antibodies

### using Herpesvirus Nucleic Acid and Amino Acid Sequences

The vaccine candidates described herein may be developed for passive immune therapy. Some portion of the protective antigens might lead to immunity via protective antibody responses. These antibodies could be useful as immediate, non-drug, therapeutic products. In passive immunotherapy, treatment may involve the delivery of biologic reagents with established immune reactivity (such as effector cells or antibodies) that can directly or indirectly mediate anti-pathogen effects and does not necessarily depend on an intact host immune system. Examples of effector cells include T lymphocytes (for example, CD8⁺ cytotoxic T-lymphocyte, CD4⁺ T-helper), killer cells (such as Natural Killer cells, lymphokine-activated killer cells), B cells, or antigen presenting cells (such as dendritic cells and macrophages) expressing the disclosed antigens. The polypeptides disclosed herein may also be used to generate antibodies or anti-idiotypic antibodies (as in U.S. Pat. No. 4,918,164) for passive immunotherapy.

In one embodiment, an effector cell is isolated and cultured. Subsequently, the effector cell is exposed or primed with an antigen of the invention. The effector cell is then reintroduced into the subject. In other embodiments, antibodies may be prepared in large quantities outside of the body and introduced into the body of a patient in need of such a treatment.

### Example 23

### Creation and Testing of Diagnostic or Drug

### Targets using Herpesvirus Nucleic Acid and Amino Acid

### Sequences

The vaccine candidates as described herein may be developed into commercial diagnostic candidates in the following manner. It is envisioned that antigens useful in raising protective immune responses may also engender rapidly detectable host responses that could be useful for identification of pathogen exposure or early-stage infection. In addition these antigens may designate key pathogen targets for developing drug-based inhibition or therapies of infection or disease.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,447,851

U.S. Pat. No. 3,791,932

U.S. Pat. No. 3,949,064

U.S. Pat. No. 4,148,876

U.S. Pat. No. 4,148,876

U.S. Pat. No. 4,174,384

U.S. Pat. No. 4,179,337

U.S. Pat. No. 4,406,885

U.S. Pat. No. 4,406,885

U.S. Pat. No. 4,444,887

U.S. Pat. No. 4,512,972

U.S. Pat. No. 4,554,101

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,676,980

U.S. Pat. No. 4,695,624

U.S. Pat. No. 4,710,111

U.S. Pat. No. 4,741,900

U.S. Pat. No. 4,816,397

U.S. Pat. No. 4,816,567

U.S. Pat. No. 4,826,687

U.S. Pat. No. 4,826,687

U.S. Pat. No. 5,112,946

U.S. Pat. No. 5,225,539

U.S. Pat. No. 5,336,603

U.S. Pat. No. 5,349,053

U.S. Pat. No. 5,359,046

U.S. Pat. No. 5,413,923

U.S. Pat. No. 5,429,599

U.S. Pat. No. 5,474,981

U.S. Pat. No. 5,474,981

U.S. Pat. No. 5,484,719

U.S. Pat. No. 5,506,121

U.S. Pat. No. 5,506,121

U.S. Pat. No. 5,523,088

U.S. Pat. No. 5,530,101

U.S. Pat. No. 5,545,806

U.S. Pat. No. 5,565,203

U.S. Pat. No. 5,565,332

U.S. Pat. No. 5,569,825

U.S. Pat. No. 5,578,453

U.S. Pat. No. 5,585,089

U.S. Pat. No. 5,585,089

U.S. Pat. No. 5,589,466

U.S. Pat. No. 5,589,466

U.S. Pat. No. 5,593,972

U.S. Pat. No. 5,605,793

U.S. Pat. No. 5,607,852

U.S. Pat. No. 5,612,487

U.S. Pat. No. 5,614,610

U.S. Pat. No. 5,614,610

U.S. Pat. No. 5,622,929

U.S. Pat. No. 5,623,057

U.S. Pat. No. 5,625,126

U.S. Pat. No. 5,633,425

U.S. Pat. No. 5,648,081

U.S. Pat. No. 5,661,016

U.S. Pat. No. 5,667,782

U.S. Pat. No. 5,670,488

U.S. Pat. No. 5,703,057

U.S. Pat. No. 5,707,644

U.S. Pat. No. 5,725,863

U.S. Pat. No. 5,747,526

U.S. Pat. No. 5,766,588

U.S. Pat. No. 5,766,588

U.S. Pat. No. 5,776,451

U.S. Pat. No. 5,783,196

U.S. Pat. No. 5,807,715

U.S. Pat. No. 5,811,238

U.S. Pat. No. 5,814,295

U.S. Pat. No. 5,814,318

U.S. Pat. No. 5,821,333

U.S. Pat. No. 5,830,721

U.S. Pat. No. 5,830,725

U.S. Pat. No. 5,834,252

U.S. Pat. No. 5,837,458

U.S. Pat. No. 5,846,225

U.S. Pat. No. 5,846,233

U.S. Pat. No. 5,851,826

U.S. Pat. No. 5,869,451

U.S. Pat. No. 5,871,982

U.S. Pat. No. 5,879,934

U.S. Pat. No. 5,885,793

U.S. Pat. No. 5,888,502

U.S. Pat. No. 5,888,773

U.S. Pat. No. 5,889,157

U.S. Pat. No. 5,889,157

U.S. Pat. No. 5,891,432

U.S. Pat. No. 5,910,306

U.S. Pat. No. 5,914,123

U.S. Pat. No. 5,916,771

U.S. Pat. No. 5,922,326

U.S. Pat. No. 5,922,326

U.S. Pat. No. 5,933,819

U.S. Pat. No. 5,939,598

U.S. Pat. No. 5,942,242

U.S. Pat. No. 5,955,331

U.S. Pat. No. 5,976,544

U.S. Pat. No. 5,976,546

U.S. Pat. No. 5,980,898

U.S. Pat. No. 5,985,318

U.S. Pat. No. 6,004,807

U.S. Pat. No. 6,005,079

U.S. Pat. No. 6,034,298

U.S. Pat. No. 6,103,493

U.S. Pat. No. 6,103,493

U.S. Pat. No. 6,129,920

U.S. Pat. No. 6,136,313

U.S. Pat. No. 6,136,320

U.S. Pat. No. 6,180,110

U.S. Pat. No. 6,194,551

U.S. Pat. No. 6,194,560

U.S. Pat. No. 6,200,959

U.S. Pat. No. 6,207,157

U.S. Pat. No. 6,210,963

U.S. Pat. No. 6,228,640

U.S. Pat. No. 6,248,334

U.S. Pat. No. 6,248,565

U.S. Pat. No. 6,254,873

U.S. Pat. No. 6,277,375 B1

U.S. Pat. No. 6,287,571

U.S. Pat. No. 6,299,881

U.S. Pat. No. 6,303,130

U.S. Pat. No. 6,309,646

U.S. Pat. No. 6,328,958

U.S. Pat. No. 6,339,068

U.S. Pat. No. 6,339,086

U.S. Pat. No. 6,348,449

U.S. Pat. No. 6,348,450

U.S. Pat. No. 6,359,054

U.S. Pat. No. 6,383,795

U.S. Pat. No. 6,406,705

U.S. Pat. No. 6,413,768

EP 0598877

EP 239,400

EP 307,434

EP 367,166

EP 439,095;

EP 519,596

EP 592,106

WO 00/42072

WO 00/75308

WO 00/75308

WO 91/06570

WO 91/09967

WO 91/10741

WO 92/01047

WO 93/21232

WO 94/04678

WO 94/25591

WO 96/04388

WO 96/33735

WO 96/33735

WO 96/34096

WO 96/34096

WO 97/33899

WO 97/34911

WO 98/16654

WO 98/24893

WO 98/24893

WO 98/46645

WO 99/16873

WO 99/16873

WO 99/50433

Ada, Mol. Immunol., 28(3):225-230, 1991.

Alberti et al., Vaccine, 16(6):608-612, 1998.

Antibodies: A Laboratory Manual, Cold Spring Harbor Laboratory, Cold Spring Harbor Press, Cold Spring Harbor, N.Y., 1988.

Arnon et al., In: Monoclonal antibodies and cancer therapy, Reisfeld et al. (eds.), 243-256, Alan R. Liss, Inc., 1985.

Ashkenazi et al., PNAS, 88:10535-10539, 1991.

Ausubel, et al., Current Protocols in Molecular Biology, 1993.

Balicki and Beutler, Medicine, 81(1):69-86. 2002.

Bangham et al., J. Mol. Biol., 13:238, 1965.

Barany and Merrifield, In: The peptides, Gross and Meienhofer (eds.), Academic Press, NY, 1-284, 1979.

Barletta et al., Res. Microbiol, 141:931-940, 1990.

Barry et al., Nature, 377(6550):632-635, 1995.

Bernstein and Stanberry, Vaccine, 17(13-14):1681-1689, 1999.

Bilbao et al., FASEB J., 11 (8):624-634, 1997.

Bowtell et al., DNA Microarrays: A Molecular Cloning Manual; Cold Spring Harbor Press, 2003

Brayton et al., Ann. N Y Acad. Sci., 849:369-371, 1998.

Caplen et al., Gene Ther., 6(3):454-459, 1999.

Carrington-Lawrence et al., J. Virol., 77(7):4237-4247, 2003.

Celluzzi and Falo, J. Invest. Dermatol., 108(5):716-720, 1997.

Coffey et al., Science, 282(5392):1332-1334, 1998.

Coney et al., Vaccine, 12(16):1545-1550, 1994.

Cunningham, J Pharmacol Toxicol Methods. July-August;44(1):291-300, 2000.

Curtiss et al., Curr. Top. Microbiol. Immunol., 146:35-49, 1989.

Da Costa et al., J. Virol. 4(17):7963-7971, 2000.

Davies et al., Biotech. and Bioengin., 74(4): 288-294, 2001.

Deamer and Nichols, Proc. Natl. Acad. Sci. USA, 80(1):165-168, 1983.

Denardo et al., Clin. Cancer Res., 4:2483-2490, 1998.

Dougan et al., Para. Immunol., 9:151-160, 1987.

Farlie et al., Acta Obstet. Gynecol. Scand., 77(1):131-133, 1998.

Farrell et al., J. Virol., 68(2):927-932, 1994.

Fell et al., J. Immunol., 146:2446-2452, 1991.

Feng et al., Nat. Biotechnol., 15(9):866-70, 1997.

Fields Virology, 3^{rd} Ed., Fields et al., (Eds.) chapter 71, 1996.

Fisher et al., Hum. Gene Ther., 7(17):2079-2087, 1996.

Froehler et al., Nucleic Acids Res., 14(13):5399-5407, 1986.

Gentz et al., Proc. Natl. Acad. Sci. USA, 86:821-824, 1989.

Gillies et al. J, Immunol. Methods, 125:191-202, 1989.

Gillies et al., PNAS, 89:1428-1432, 1992.

Glenn et al., Exp. Opin. Invest. Drugs, 8:797-805, 1999.

Gnant et al., Cancer Res., 59(14):3396-403, 1999.

Goodman and Gilman's The Pharmacological Basis of Therapeutics, 6th Ed., Goodman et al., (Eds.), 1080-1248; Macmillan Publishing Co., NY, 1980.

Gregoriadis, Drug Carriers In Biology And Medicine, G. Gregoriadis (ed.), 287-341, 1979.

Hale, Res. Microbiol., 141(7-8):913-919, 1990.

Hansson et al., J. Mol. Biol., 287:265, 1999.

Harayama, Trends Biotechnol., 16:76, 1998.

Harlow and Lane, In: Antibodies, A Laboratory manual, Cold Spring Harbor Laboratory, 1988.

Hellstrom et al., In: Controlled drug delivery, 2d Ed., Robinson et al. (eds.), 623-653, Marcel Dekker, Inc., 1987.

Holzer et al. Virology, 253(1):107-114, 1999.

Hosaka et al., J. Virol., 46(3):1014-1017, 1983.

Huang et al., Virology, 97:212-217, 1979.

Imai et al., Nephrologie, 19(7):397-402, 1998.

Inagaki-Ohara et al., Vaccine, 20(1-2):98-104, 2001.

Janson and Ryden, In: Protein purification: principles, high-resolution methods, and applications, 2d Ed., 1998.

Jespers et al., Bio/technology, 12:899-903, 1988.

Jones et al., Proc. Natl. Acad. Sci. USA, 93(21):11327-11333, 1996.

Kaneda, Adv. Drug Deliv. Rev., 43(2-3):197-205, 2000.

Keadle et al., J. Infectious Dis., 176, 331-338, 1997.

Klein et al., Nature, 327:70-73, 1987.

Kleymann et al., Nat. Med., 8(4):392-398, 2002.

Knappik et al., Biotechniques, 17(4):754-761, 1994.

Kobayishi et al., Immunochemistry, 10:73, 1973.

Kyte and Doolittle, J Mol Biol, 157(1):105-32, 1982.

Leung and Sacks, Drugs, 60(6):1329-1352, 2000.

Levine et al, Microecol. Ther., 19:23-32, 1990.

Liu and Huang, J. Control Release, 78(1-3):259-66, 2002.

Lonberg and Huszar, Int. Rev. Immunol., 13:65-93, 1995.

Lorenzo and Blasco, BioTechniques, 24:308, 1998.

Lowrie et al., Vaccine, 12(16):1537-1540, 1994.

Lundstrom, J. Recept Signal Transduct. Res., 19(1-4):673-686, 1999.

Manoutcharian et al., Immunol. Lett., 62(3):131-136, 1998.

Marshak et al., In: Strategies for protein purification and characterization, A Laboratory Course Manual, Cold Spring Harbor Laboratory, 1996.

Merrifield, Science, 232:341-347, 1986.

Moore et al., Vaccine, 20(1-2), 2001.

Moore, Trends Pharmacol. Sci., 15(4):124-129, 1994.

Morris et al., Gastroenterol, 103:699-702, 1992.

Morrison, Science, 229:1202, 1985.

Mullis et al., Biotechnology, 24:17-27, 1992.

Mullis, Sci. Am., 262(4):56-61, 64-65, 1990.

Munson and Pollard, Anal. Biochem., 107:220, 1980.

Muldermans et al., Trends Biochem. Sci., 26:230, 2001.

Naramura et al., Immunol. Lett., 39:91-99, 1994.

Navarro et al., Virology, 186(1), 99-112, 1992.

Nesburn et al., Invest. Ophthal. Visual Sci., 39(7):1163-1170, 1998.

Nesburn et al., J. Virology, 68(8):5084-5092, 1994.

Nesburn et al., Virology, 252(1), 200-209, 1998.

Neumann et al., Proc. Natl. Acad. Sci. USA, 96(16):9345-9350, 1999.

Nicolau et al., Methods Enzymol., 149:157-176, 1987.

Nuttall et al., Cur. Pharm. Biotech., 1:253, 2000.

Ol et al., BioTechniques, 4-214, 1986.

Padlan, Molec. Immunol., 28(4/5):489-498, 1991.

Patten et al., Curr. Opinion Biotechnol., 8:724-33, 1997.

Peterson et al., Bioconjug. Chem., 10:553, 1999.

Piedrafita et al., J. Immunol., 163(3):1467-1472, 1999.

Ravid et al., In: DNA transfer to cultured cells, Wiley and Sons, 1998

Reichmann and Muyldermans, J. Immunol. Meth., 231:25, 1999.

Remington's Pharmaceutical Sciences, 15^{th} ed., pages 1035-1038 and 1570-1580, Mack Publishing Company, Easton, Pa., 1980.

Riechmann et al., Nature, 332:323-327, 1988.

Robbins and Ghivizzani, Trends Biotech., 16(1):35-40, 1998.

Roguska et al., Proc. Natl. Acad. Sci. USA, 91:969-973, 1994.

Rouse et al., J. Virol., 68(9):5685-5689, 1994.

Sambrook et al., Molecular Cloning: A Laboratory Manual, Cold Spring Harbor Laboratory; 3rd edition, 2001.

Sanford et al., Technique, 3:3-16, 1991.

Shields et al., J. Biol. Chem., 277(30):26733-26740, 2001.

Slack, In: Oxford Textbook of Medicine, 2d Ed., Weatherall et al., (Eds.) 1:5.35, Oxford University Press, Oxford/Melbourne/New York, 1987.

Smooker et al., Vaccine 18:2533-2540, 2000.

Stanberry et al., Clinical Infect. Dis., 30(3):549-566, 2000.

Stanberry et al., In: Viral infections of humans: epidemiology and control, Evans et al., (Eds.), Plenum Medical Book Company, NY, 419-454, 1997.

Steinman et al., Hum Immunol, 60(7):562-567, 1999

Stemmer et al., Gene, 154(1):49-53, 1995.

Stewart and Young, In: Solid phase peptide synthesis, 2d. ed., Pierce Chemical Co. 1984.

Studnicka et al., Protein Eng., 7(6):805-814,1994.

Sykes and Johnston, DNA Cell. Biol., 18:521-531, 1999.

Sykes and Johnston, Nat. Biotechnol., 17(4):355-359, 1999.

Szoka and Papahadjopoulos, Proc. Natl. Acad. Sci. USA, 75:4194-4198, 1978.

Takahashi et al., J. Immunol., 6:1567-1574, 1994

Tam et al., J. Am. Chem. Soc., 105:6442, 1983.

Tang et al., Nature, 356:152-154, 1992.

Thorpe et al., Immunol. Rev., 65:119-138, 1982.

Thorpe, In: Monoclonal antibodies '84: Biological and clinical applications, Pinchera et al. (eds.), 475-506, 1985.

Van Damme et al., Gastroenterology, 103(2):520-531, 1992.

Vil et al., PNAS, 89:11337-11341, 1992.

Viret et al., Mol. Microbiol., 7(2):239-252, 1993.

Watt et al., Proc. Natl Acad. Sci., 83(2):3166-3170, 1986.

Whitley and Miller, Viral Immunol., 14(2):111-118, 2001.

Whitley and Miller, Viral. Immunol., 14(2), 111-118, 2001.

Whitley and Roizman, Lancet., 357(9267):1513-1518, 2001.

Whitley et al., Clin. Infect. Dis., 26(3):541-553, 1998.

Wilson et al., Cell, 37:767, 1984.

Wu et al., Genomics, 4:560-569, 1989.

Xiang et al., J. Virol, 199(1):132-140, 1994.

Zheng et al., J. Immunol., 154:5590-5600, 1995.

Zimmerman et al., Nucl. Med. Biol., 26:943-50, 1999.

What is claimed is:

1. A method of immunizing a subject comprising providing to the subject a pharmaceutical composition in an amount effective to induce an immune response, the pharmaceutical composition comprising at least one herpesvirus antigen or fragment thereof.

2. The method of claim 1, wherein the herpesvirus antigen or fragment thereof is further defined as a HSV-1 antigen or fragment thereof.

3. The method of claim 1, wherein the at least one herpesvirus antigen has an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof.

4. The method of claim 1, wherein the subject is immunized against an animal herpesvirus.

5. The method of claim 4, wherein the subject is immunized against a human herpesvirus.

6. The method of claim 5, wherein the subject is immunized against HSV-1, HSV-2, or Varicella Zoster Virus.

7. The method of claim 5, wherein the subject is immunized against HSV-1.

8. The method of claim 5, wherein the subject is immunized against HSV-2.

9. The method of claim 4, wherein the subject is immunized against a cercopithecine, bovine or canine herpesvirus.

10. The method of claim 1, wherein the method of providing at least one herpesvirus antigen(s) comprises:
(a) preparing a pharmaceutical composition comprising at least one polynucleotide encoding a polypeptide having an amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof;
(b) administering one or more polynucleotides in a pharmaceutically acceptable carrier into the subject; and
(c) expressing one or more herpesvirus antigens in the subject.

11. The method of claim 10, wherein the polynucleotide is an expression vector.

12. The method of claim 11, wherein the expression vector is a genetic immunization vector.

13. The method of claim 11, wherein the expression vector is a linear expression element or circular expression element expression system.

14. The method of claim 10, wherein the polynucleotide sequence is set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or a fragment thereof.

15. The method of claim 14, wherein the polynucleotide is administered by a intramuscular injection, epidermal injection or particle bombardment.

16. The method of claim 14, wherein the polynucleotide is administered by intravenous, subcutaneous, intralesional, intraperitoneal, intradermal, oral, or other mucosal or inhaled routes of administration.

17. The method of claim 16, wherein a second administration is given at least about three weeks after the first administration.

18. The method of claim 10, wherein at least two polynucleotides encoding different herpesvirus antigens or fragments thereof are administered to a subject.

19. The method of claim 1, wherein at least two different herpesvirus antigens, or fragments thereof, are provided in an amount effective to induce an immune response.

20. An isolated polynucleotide comprising a sequence having at least 17 contiguous nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ BD NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or its complement.

21. The polynucleotide of claim 20, further defined as comprising a sequence having least 50 contiguous nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ BD NO:105, SEQ ID NO:107, SEQ ID NO:109, SEQ ID NO:111, SEQ ID NO:113 or SEQ ID NO:115, or its complement.

22. The polynucleotide of claim 21, further defined as comprising a sequence having all nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or its complement.

23. The polynucleotide of claim 20, further defined as being comprised in a vector.

24. The polynucleotide of claim 20, further defined as being comprised in a pharmaceutical composition.

25. The polynucleotide of claim 20, further defined as being comprised in a vaccine.

26. An isolated polypeptide having at least 5 consecutive amino acids of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116.

27. The polypeptide of claim 26, wherein the polypeptide comprises at least 20 consecutive amino acids of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116.

28. The polypeptide of claim 27, further defined as having an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116.

29. The polypeptide of claim 26, further defined as being comprised in a pharmaceutical composition.

30. The polypeptide of claim 26, further defined as being comprised in a vaccine.

31. The polypeptide of claim 28, further defined as a recombinant polypeptide.

32. A vaccine composition comprising at least one herpesvirus antigen or fragment thereof or at least one polynucleotide encoding a herpesvirus antigen or a fragment thereof.

33. The vaccine composition of claim 32, further defined as a genetic vaccine, a polypeptide vaccine, a cell-mediated vaccine, an attenuated pathogen vaccine, a live-vector vaccine, an edible vaccine, a killed pathogen vaccine, a purified sub-unit vaccine, a conjugate vaccine, a virus-like particle vaccine, or a humanized antibody vaccines.

34. The vaccine composition of claim 33, further defined as comprising a polynucleotide encoding at least one herpesvirus antigen or fragment thereof.

35. The vaccine composition of claim 33, further defined as comprising at least one herpesvirus antigen or a fragment thereof.

36. The vaccine composition of claim 32, further defined as comprising at least one polynucleotide encoding a herpesvirus antigen or fragment thereof.

37. The vaccine composition of claim 36, further defined as comprising at least two polynucleotides encoding different herpesvirus antigens or fragments thereof.

38. The vaccine composition of claim 36, wherein the polynucleotide encoding the herpesvirus antigen or fragment thereof encodes a polypeptide comprising the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof.

39. The vaccine composition of claim 36, wherein the polynucleotide encoding a herpesvirus antigen or fragment thereof comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or a fragment thereof.

40. The vaccine composition of claim 32, further defined as comprising at least one herpesvirus antigen or fragment thereof in a pharmaceutically acceptable carrier.

41. The vaccine composition of claim 40, further defined as comprising at least two different herpesvirus antigens or fragments thereof in a pharmaceutically acceptable carrier.

42. The vaccine composition of claim 40, wherein the herpesvirus antigen or fragments thereof has an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or fragments thereof.

43. A method of screening for at least one test polypeptide or test polynucleotide encoding a polypeptide for an ability to produce an immune response comprising:
(i) obtaining at least one test polypeptide or test polynucleotide by (a) modifying the amino acid sequence of a known antigenic polypeptide or polynucleotide sequence of a polynucleotide encoding a known antigenic polypeptide; (b) obtaining a homolog of a known antigenic sequence of a polynucleotide encoding such a homolog, or (c) obtaining a homolog of a known antigenic sequence or a polynucleotide encoding such a homolog and modifying the amino acid sequence of the homolog or the polynucleotide sequence of the polynucleotide encoding such a homolog; and
(ii) testing the test polypeptide or test polynucleotide under appropriate conditions to determine whether the test polypeptide is antigenic or the test polynucleotide encodes an antigenic polypeptide.

44. The method of claim 43, further defined as comprising obtaining a test polypeptide.

45. The method of claim 44, wherein obtaining the test polypeptide comprises modifying the amino acid sequence or obtaining a homolog of a least one polypeptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116 or fragment thereof.

46. The method of claim 43, further defined as comprising obtaining a test polynucleotide.

47. The method of claim 46, wherein obtaining the test polynucleotide comprises modifying the polynucleotide sequence of at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or fragment thereof.

48. The method of claim 43, further comprising identifying at least one test polypeptide as being antigenic or at least one test polynucleotide as encoding an antigenic polypeptide.

49. The method of claim 48, further comprising placing the identified antigenic polypeptide or the polynucleotide encoding an antigenic polypeptide in a pharmaceutical composition.

50. The method of claim 48, further comprising using the identified antigenic polypeptide or polynucleotide encoding an antigenic polypeptide to vaccinate a subject.

51. The method of claim 50, wherein the subject is vaccinated against a herpesvirus.

52. The method of claim 51, wherein the herpesvirus is HSV-1.

53. The method of claim 50, wherein the subject is vaccinated against a non-herpesvirus disease.

54. A method of preparing a vaccine comprising obtaining an antigenic polypeptide or a polynucleotide encoding an antigenic polypeptide as determined to be antigenic by the method of claim 43, and placing the polypeptide or polynucleotide in a vaccine composition.

55. A method of vaccinating a subject comprising preparing a vaccine of claim 54 and vaccinating a subject with the vaccine.

56. A method of treating a subject infected with a pathogen comprising administering a vaccine composition comprising at least one herpesvirus antigen or fragment thereof, or at least one polynucleotide encoding a herpesvirus antigen or a fragment thereof.

57. The method of claim 56, wherein the vaccine composition is a genetic vaccine, a polypeptide vaccine, a cell-mediated vaccine, an attenuated pathogen vaccine, a live-vector vaccine, an edible vaccine, a killed pathogen vaccine, a purified sub-unit vaccine, a conjugate vaccine, a virus-like particle vaccine, or a humanized antibody vaccine.

58. The method of claim 57, wherein the vaccine composition comprises a polynucleotide encoding at least one herpesvirus antigen or fragment thereof.

59. The method of claim 57, wherein the vaccine composition comprises at least one herpesvirus antigen or fragment thereof.

60. The method of claim 58, wherein the polynucleotide encoding the herpesvirus antigen or fragment thereof encodes a polypeptide comprising the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof.

61. The method of claim 58, wherein the polynucleotide encoding a herpesvirus antigen or fragment thereof comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71; SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113 or SEQ ID NO:115, or a fragment thereof.

62. A method of raising a therapeutic immune response against reactivation disease comprising administering a vaccine composition comprising at least one herpesvirus antigen or fragment thereof, or at least one polynucleotide encoding a herpesvirus antigen or a fragment thereof.

63. The method of claim 62, wherein the herpesvirus antigen comprises an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof.

64. A method of passive immunization comprising administering at least one antigen binding agent reactive to one or more herpesvirus antigen to a subject.

65. The method of claim 64, wherein the herpesvirus antigen comprises an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, and/or SEQ ID NO:116, or a fragment thereof.

66. A method for vaccination comprising administering a priming dose of a herpesvirus vaccine composition.

67. The method of claim 66, wherein the priming dose is followed by a boost dose.

68. The method of claim 66, wherein the vaccine composition is administered at least once.

69. The method of claim 68, wherein the vaccine is administered at least twice.

70. The method of claim 69, wherein the method comprises (a) administering at least one nucleic acid vaccine composition and then (b) administering at least one polypeptide vaccine composition.

71. The method of claim 69, wherein the method comprises the steps of (a) administering at least one polypeptide vaccine composition and then (b) administering at least one nucleic acid vaccine composition.

72. The method of claim 67, wherein the method comprises (a) administering at least one nucleic acid vaccine composition and then (b) administering at least one polypeptide vaccine composition.

73. The method of claim 67, wherein the method comprises the steps of (a) administering at least one polypeptide vaccine composition and then (b) administering at least one nucleic acid vaccine composition.


SEQUENCE LISTING (116 sequences)

SEQ ID NO: 1 - DNA, 342 bp, Herpes Virus
gccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctc

SEQ ID NO: 2 - protein, 114 aa, Herpes Virus
AVPTAQSQVTSTPNSEPAVRSAPAAAPPPPPAGGPPPSCSLLLRQWLHVPESASDDDDDDDWPDSPPPEPAPEARPTAAAPRPRPPPPGVGPGGGADPSHPPSRPFRLPPRLAL

SEQ ID NO: 3 - DNA, 747 bp, Herpes Virus
atggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaa

SEQ ID NO: 4 - protein, 248 aa, Herpes Virus
MARRRRHRGPRRPRPPGPTGAVPTAQSQVTSTPNSEPAVRSAPAAAPPPPPAGGPPPSCSLLLRQWLHVPESASDDDDDDDWPDSPPPEPAPEARPTAAAPRPRPPPPGVGPGGGADPSHPPSRPFRLPPRLALRLRVTAEHLARLRLRRAGGEGAPEPPATPATPATPATPATPARVRFSPHVRVRHLVVWASAARLARRGSWARERADRARFRRRVAEAEAVIGPCLGPEARARALARGAGPANSV

SEQ ID NO: 5 - DNA, 591 bp, Herpes Virus
ccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacga

SEQ ID NO: 6 - protein, 197 aa, Herpes Virus
PSTEYVIRSRVAREVGDILKVPCVPLPSDDLDWRYETPSAINYALIDGIFLRYHCPGLDTVLWDRHAQKAYWVNPFLFVAGFLEDLSYPAFPANTQETETRLALYKEIRQALDSRKQAASHTPVKAGCVNFDYSRTRRCVGRQDLGPTNGTSGRTPVLPPDDEAGLQPKPLTTPPPIIATSDPTPRRDAATKSRRRR

SEQ ID NO: 7 - DNA, 675 bp, Herpes Virus
atggggattttgggttgggtcgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacgaccccactcccggcgcctctaa

SEQ ID NO: 8 - protein, 224 aa, Herpes Virus
MGILGWVGLIAVGVLCVRGGLPSTEYVIRSRVAREVGDILKVPCVPLPSDDLDWRYETPSAINYALIDGIFLRYHCPGLDTVLWDRHAQKAYWVNPFLFVAGFLEDLSYPAFPANTQETETRLALYKEIRQALDSRKQAASHTPVKAGCVNFDYSRTRRCVGRQDLGPTNGTSGRTPVLPPDDEAGLQPKPLTTPPPIIATSDPTPRRDAATKSRRRRPHSRRL

SEQ ID NO: 9 - DNA, 1292 bp, Herpes Virus
cagctagacggacagaaacccggcccgccgcaccttcagcaacccggggaccgaccagccgttccagggagggccgaggcctttttaaattttacgtctatgcacggggtgcagccaatccttaagcgcatccgagagctctcgcaacaacagctcgacggagcgcaagtgccccatctgcagtggttccgggacgtggcggccttagagtcccccgcaggcctgcccctcagggagtttccgttcgcggtgtatcttatcaccggcaacgctggctccggaaagagcacgtgcgtgcagacaatcaacgaggtcttggactgtgtggtgacgggcgccacgcgcattgcggcccaaaacatgtacgccaaactctcgggcgcctttctcagccgacccatcaacaccatctttcatgaatttgggtttcgcgggaatcacgtccaggcccaactgggacagtacccgtacaccctgaccagcaaccccgcctcgctggaggacctgcagcgacgagatctgacgtactactgggaggtgattttggacctcacgaagcgcgccctggccgcctccgggggcgaggagttgcggaacgagtttcgcgccctggccgccctggaacggaccctggggttggccgagggcgccctgacgcggttggccccggccacccacggggcgctgccggcctttacccgcagcaacgtgatcgtcatcgacgaggccgggctccttgggcgtcacctcctcacggccgtggtgtattgctggtggatgattaacgccctgtaccacaccccccagtacgcggcccgcctgcggcccgtgttggtgtgtgtgggctcgccgacgcagacggcgtccctggagtcgaccttcgagcaccagaaactgcggtgttccgtccgccagagcgagaacgtgctcacgtacctcatctgcaaccgcacgctgcgcgagtacgcccgcctctcgtatagctgggccatttttattaacaacaaacggtgcgtcgagcacgagttcggtaacctcatgaaggtgctggagtacggcctgcccatcaccgaggagcacatgcagttcgtggatcgcttcgtcgtcccggaaaactacatcaccaaccccgccaacctccccggctggacgcggctgttctcctcccacaaagaggtgagcgcgtacatggccaagctccacgcctacctgaaggtgacccgtgagggggagttcgtcgtgttcaccctccccgtgcttacgttcgtgtcggtcaaggagtttgacgaata

SEQ ID NO: 10 - protein, 430 aa, Herpes Virus
QLDGQKPGPPHLQQPGDRPAVPGRAEAFLNFTSMHGVQPILKRIRELSQQQLDGAQVPHLQWFRDVAALESPAGLPLREFPFAVYLITGNAGSGKSTCVQTINEVLDCVVTGATRIAAQNMYAKLSGAFLSRPINTIFHEFGFRGNHVQAQLGQYPYTLTSNPASLEDLQRRDLTYYWEVILDLTKRALAASGGEELRNEFRALAALERTLGLAEGALTRLAPATHGALPAFTRSNVIVIDEAGLLGRHLLTAVVYCWWMINALYHTPQYAARLRPVLVCVGSPTQTASLESTFEHQKLRCSVRQSENVLTYLICNRTLREYARLSYSWAIFINNKRCVEHEFGNLMKVLEYGLPITEEHMQFVDRFVVPENYITNPANLPGWTRLFSSHKEVSAYMAKLHAYLKVTREGEFVVFTLPVLTFVSVKEFDE

SEQ ID NO: 11 - DNA, 2649 bp, Herpes Virus
atggcggcggccggcggggagcgccagctagacggacagaaacccggcccgccgcaccttcagcaacccggggaccgaccagccgttccagggagggccgaggcctttttaaattttacgtctatgcacggggtgcagccaatccttaagcgcatccgagagctctcgcaacaacagctcgacggagcgcaagtgccccatctgcagtggttccgggacgtggcggccttagagtcccccgcaggcctgcccctcagggagtttccgttcgcggtgtatcttatcaccggcaacgctggctccggaaagagcacgtgcgtgcagacaatcaacgaggtcttggactgtgtggtgacgggcgccacgcgcattgcggcccaaaacatgtacgccaaactctcgggcgcctttctcagccgacccatcaacaccatctttcatgaatttgggtttcgcgggaatcacgtccaggcccaactgggacagtacccgtacaccctgaccagcaaccccgcctcgctggaggacctgcagcgacgagatctgacgtactactgggaggtgattttggacctcacgaagcgcgccctggccgcctccgggggcgaggagttgcggaacgagtttcgcgccctggccgccctggaacggaccctggggttggccgagggcgccctgacgcggttggccccggccacccacggggcgctgccggcctttacccgcagcaacgtgatcgtcatcgacgaggccgggctccttgggcgtcacctcctcacggccgtggtgtattgctggtggatgattaacgccctgtaccacaccccccagtacgcggcccgcctgcggcccgtgttggtgtgtgtgggctcgccgacgcagacggcgtccctggagtcgaccttcgagcaccagaaactgcggtgttccgtccgccagagcgagaacgtgctcacgtacctcatctgcaaccgcacgctgcgcgagtacgcccgcctctcgtatagctgggccatttttattaacaacaaacggtgcgtcgagcacgagttcggtaacctcatgaaggtgctggagtacggcctgcccatcaccgaggagcacatgcagttcgtggatcgcttcgtcgtcccggaaaactacatcaccaaccccgccaacctccccggctggacgcggctgttctcctcccacaaagaggtgagcgcgtacatggccaagctccacgcctacctgaaggtgacccgtgagggggagttcgtcgtgttcaccctccccgtgcttacgttcgtgtcggtcaaggagtttgacgaataccgacggctgacacaccagcccggcctgacgattgaaaagtggctcacggccaacgccagccgcatcaccaactactcgcagagccaggaccaggacgcggggcacatgcgctgcgaggtgcacagcaaacagcagctggtcgtggcccgcaacgacgtcacttacgtcctcaacagccagatcgcggtgaccgcgcgcctgcgaaaactggtttttgggtttagtgggacgttccgggccttcgaggcagtgttgcgtgacgacagctttgtaaagactcagggggagacttcggtggagtttgcctacaggttcctgtcgcggctcatatttagcgggcttatctccttttacaactttctgcagcgcccgggcctggatgcgacccagaggaccctcgcctacgcccgcatgggagaactaacggcggagattctgtctctgcgccccaaatcttcgggggtgccgacgcaggcgtcggtaatggccgacgcaggcgcccccggcgagcgtgcgtttgattttaagcaactggggccgcgggacgggggcccggacgattttcccgacgacgacctcgacgttattttcgcggggctggacgaacaacagctcgacgtgttttactgccactacacccccggggaaccggagaccaccgccgccgttcacacccagtttgcgctgctgaagcgggccttcctcgggagattccgaatcctccaagagctcttcggggaggcatttgaagtcgccccctttagcacgtacgtggacaacgttatcttccggggctgcgagatgctgaccggctcgccgcgcggggggctgatgtccgtcgccctgcagacggacaattatacgctcatgggatacacgtacgcacgggtgtttgcctttgcggacgagctgcggaggcggcacgcgacggccaacgtggccgagttactggaagaggcccccctgccttacgtggtcttgcgggaccaacacggcttcatgtccgtcgtcaacaccaacatcagcgagtttgtcgagtccattgactctacggagctggccatggccataaacgccgactacggcatcagctccaagcttgccatgaccatcacgcgctcccagggccttagcctggacaaggtcgccatctgctttacgcccggcaacctgcgcctcaacagcgcgtacgtggccatgtcccgcaccacctcctccgaattccttcgcatgaacttaaatccgctccgggagcgccacgagcgcgatgacgtcattagtgagcacatactatcggctctgcgcgatccgaacgtggtcattgtctattaa

SEQ ID NO: 12 - protein, 882 aa, Herpes Virus
MAAAGGERQLDGQKPGPPHLQQPGDRPAVPGRAEAFLNFTSMHGVQPILKRIRELSQQQLDGAQVPHLQWFRDVAALESPAGLPLREFPFAVYLITGNAGSGKSTCVQTINEVLDCVVTGATRIAAQNMYAKLSGAFLSRPINTIFHEFGFRGNHVQAQLGQYPYTLTSNPASLEDLQRRDLTYYWEVILDLTKRALAASGGEELRNEFRALAALERTLGLAEGALTRLAPATHGALPAFTRSNVIVIDEAGLLGRHLLTAVVYCWWMINALYHTPQYAARLRPVLVCVGSPTQTASLESTFEHQKLRCSVRQSENVLTYLICNRTLREYARLSYSWAIFINNKRCVEHEFGNLMKVLEYGLPITEEHMQFVDRFVVPENYITNPANLPGWTRLFSSHKEVSAYMAKLHAYLKVTREGEFVVFTLPVLTFVSVKEFDEYRRLTHQPGLTIEKWLTANASRITNYSQSQDQDAGHMRCEVHSKQQLVVARNDVTYVLNSQIAVTARLRKLVFGFSGTFRAFEAVLRDDSFVKTQGETSVEFAYRFLSRLIFSGLISFYNFLQRPGLDATQRTLAYARMGELTAEILSLRPKSSGVPTQASVMADAGAPGERAFDFKQLGPRDGGPDDFPDDDLDVIFAGLDEQQLDVFYCHYTPGEPETTAAVHTQFALLKRAFLGRFRILQELFGEAFEVAPFSTYVDNVIFRGCEMLTGSPRGGLMSVALQTDNYTLMGYTYARVFAFADELRRRHATANVAELLEEAPLPYVVLRDQHGFMSVVNTNISEFVESIDSTELAMAINADYGISSKLAMTITRSQGLSLDKVAICFTPGNLRLNSAYVAMSRTTSSEFLRMNLNPLRERHERDDVISEHILSALRDPNVVIVY

SEQ ID NO: 13 - DNA, 1011 bp, Herpes Virus
caactgttagacccgcccgcggccgtcgggcccgtctggacggcgcggttttgcttccccggacttcgcgcccagctcctggcggccctggccgacctcggggggagcgggctggcggacccccacggccggacgggcctagcaagactggacgcgctggtggtggccgctccctcagagccctgggccggggccgtcttggagcgcctggtcccggacacgtgcaacgcctgccctgcgctgcggcagctcctgggtggggtaatggccgccgtctgcctgcagatcgaggagacggccagctcggtgaagttcgcggtctgcgggggcgatgggggtgcgttctggggtgtctttaacgtggacccccaagacgcggatgcggcttccggggtgatcgaggacgcccggcgggccatcgagacggccgtgggagccgtgcttagggccaacgccgtccggctgcggcacccactgtgcctggccctcgagggcgtctacacccacgcagtcgcctggagccaggcgggagtgtggttctggaactcccgcgacaacactgaccatcttgggggatttcctctccgcgggcccgcgtacaccacggcggcaggggtcgtacgcgacacgctgcgacgggtcctgggcctgacaacggcatgcgtgccggaggaggacgcactcacggcccggggccttatggaggacgcctgcgaccgccttatcttggacgcgtttaataaacggttggacgcggagtactggagcgttcgggtgtccccctttgaggccagcgaccccttgccccccactgccttccgcggcggcgccttgctggacgcagagcactactggcggcgcgtcgtgcgtgtctgtcccggaggcggggagtcggtcggcgtccccgtcgatctatacccgcggccccttgtgctcccccccgtggactgcgctcatcacctgcgcgaaatcctgcgcgagattgagttggtgtttaccggggtgctggcg

SEQ ID NO: 14 - protein, 337 aa, Herpes Virus
QLLDPPAAVGPVWTARFCFPGLRAQLLAALADLGGSGLADPHGRTGLARLDALVVAAPSEPWAGAVLERLVPDTCNACPALRQLLGGVMAAVCLQIEETASSVKFAVCGGDGGAFWGVFNVDPQDADAASGVIEDARRAIETAVGAVLRANAVRLRHPLCLALEGVYTHAVAWSQAGVWFWNSRDNTDHLGGFPLRGPAYTTAAGVVRDTLRRVLGLTTACVPEEDALTARGLMEDACDRLILDAFNKRLDAEYWSVRVSPFEASDPLPPTAFRGGALLDAEHYWRRVVRVCPGGGESVGVPVDLYPRPLVLPPVDCAHHLREILREIELVFTGVLA

SEQ ID NO: 15 - DNA, 2253 bp, Herpes Virus
atggacaccgcagatatcgtgtgggtggaggagagcgtcagcgccattaccctttacgcggtatggctgcccccccgcgctcgcgagtacttccacgccctggtgtattttgtatgtcgcaacgccgcaggggagggtcgcgcgcgctttgcggaggtctccgtcaccgcgacggagctgcgggatttctacggctccgcggacgtctccgtccaggccgtcgtggcggccgcccgcgccgcgacgacgccggccgcctccccgctggagcccctggagaacccgactctgtggcgggcgctgtacgcgtgcgtcctggcggccctggagcgccagaccgggccggtggccctgttcgccccgctgcgtatcggctcggacccacgcacgggactggtggtgaaagttgagagagcgtcgtggggcccgcccgccgcccctcgcgccgctctcctggtcgcggaggccaacattgacatcgaccctatggccctggcggcgcgcgttgccgagcatcccgacgcgcggctggcgtgggcgcgcctggcggccattcgcgacaccccccagtgcgcgtccgccgcttcgctgaccgttaacatcaccaccggaaccgcgctatttgcgcgcgaataccagactcttgcgtttccgccgatcaagaaggagggcgcgttcggggacctggtcgaggtgtgcgaggtgggcctgcggccacgcgggcacccgcaacgagtcacggcacgggtgctgctgccccgcgattacgactactttgtaagcgccggcgagaagttctccgcgccggcgctcgtcgcccttttccggcagtggcataccacggtccacgccgcccccggggccctggcccccgtctttgcctttctggggcccgagtttgaggtccgggggggacccgtcccgtactttgccgtcctggggtttccgggttggcccacgttcaccgtgccggccacggccgagtcggcacgggacctggtgcgcggggccgcggccgcttacgccgcgctcctgggggcctggcccgcggtgggggccagggtcgtcctccccccgcgagcctggcccggcgtggcctcggcggcagccggatgcctcctgcccgcggtgcgggaggcggtggcgcggtggcatcccgccactaaaatcatccaactgttagacccgcccgcggccgtcgggcccgtctggacggcgcggttttgcttccccggacttcgcgcccagctcctggcggccctggccgacctcggggggagcgggctggcggacccccacggccggacgggcctagcaagactggacgcgctggtggtggccgctccctcagagccctgggccggggccgtcttggagcgcctggtcccggacacgtgcaacgcctgccctgcgctgcggcagctcctgggtggggtaatggccgccgtctgcctgcagatcgaggagacggccagctcggtgaagttcgcggtctgcgggggcgatgggggtgcgttctggggtgtctttaacgtggacccccaagacgcggatgcggcttccggggtgatcgaggacgcccggcgggccatcgagacggccgtgggagccgtgcttagggccaacgccgtccggctgcggcacccactgtgcctggccctcgagggcgtctacacccacgcagtcgcctggagccaggcgggagtgtggttctggaactcccgcgacaacactgaccatcttgggggatttcctctccgcgggcccgcgtacaccacggcggcaggggtcgtacgcgacacgctgcgacgggtcctgggcctgacaacggcatgcgtgccggaggaggacgcactcacggcccggggccttatggaggacgcctgcgaccgccttatcttggacgcgtttaataaacggttggacgcggagtactggagcgttcgggtgtccccctttgaggccagcgaccccttgccccccactgccttccgcggcggcgccttgctggacgcagagcactactggcggcgcgtcgtgcgtgtctgtcccggaggcggggagtcggtcggcgtccccgtcgatctatacccgcggccccttgtgctcccccccgtggactgcgctcatcacctgcgcgaaatcctgcgcgagattgagttggtgtttaccggggtgctggcgggagtatggggcgagggggggaagtttgtgtatccctttgacgacaagatgtcgtttctgtttgcctga

SEQ ID NO: 16 - protein, 750 aa, Herpes Virus
MDTADIVWVEESVSAITLYAVWLPPRAREYFHALVYFVCRNAAGEGRARFAEVSVTATELRDFYGSADVSVQAVVAAARAATTPAASPLEPLENPTLWRALYACVLAALERQTGPVALFAPLRIGSDPRTGLVVKVERASWGPPAAPRAALLVAEANIDIDPMALAARVAEHPDARLAWARLAAIRDTPQCASAASLTVNITTGTALFAREYQTLAFPPIKKEGAFGDLVEVCEVGLRPRGHPQRVTARVLLPRDYDYFVSAGEKFSAPALVALFRQWHTTVHAAPGALAPVFAFLGPEFEVRGGPVPYFAVLGFPGWPTFTVPATAESARDLVRGAAAAYAALLGAWPAVGARVVLPPRAWPGVASAAAGCLLPAVREAVARWHPATKIIQLLDPPAAVGPVWTARFCFPGLRAQLLAALADLGGSGLADPHGRTGLARLDALVVAAPSEPWAGAVLERLVPDTCNACPALRQLLGGVMAAVCLQIEETASSVKFAVCGGDGGAFWGVFNVDPQDADAASGVIEDARRAIETAVGAVLRANAVRLRHPLCLALEGVYTHAVAWSQAGVWFWNSRDNTDHLGGFPLRGPAYTTAAGVVRDTLRRVLGLTTACVPEEDALTARGLMEDACDRLILDAFNKRLDAEYWSVRVSPFEASDPLPPTAFRGGALLDAEHYWRRVVRVCPGGGESVGVPVDLYPRPLVLPPVDCAHHLREILREIELVFTGVLAGVWGEGGKFVYPFDDKMSFLFA

SEQ ID NO: 17 - DNA, 252 bp, Herpes Virus
tgccgaaacaacgtcctcatcaccgacgacggggaggtcgtctcgctgaccgcccacgactttgacgtcgtggatatcgagtccgaagaggaaggtaatttctacgtgcccccggatatgcgcggggttacgcgggccccggggagacagcgcctgcgttcatcggaccccccctcgcgccacactcaccggcggacccccggaggcgcctgccccgccacccagtttccaccccccatgtccgatagcgaa

SEQ ID NO: 18 - protein, 84 aa, Herpes Virus
CRNNVLITDDGEVVSLTAHDFDVVDIESEEEGNFYVPPDMRGVTRAPGRQRLRSSDPPSRHTHRRTPGGACPATQFPPPMSDSE

SEQ ID NO: 19 - DNA, 291 bp, Herpes Virus
atgggcctctcgttctccggggcccggccctgctgctgccgaaacaacgtcctcatcaccgacgacggggaggtcgtctcgctgaccgcccacgactttgacgtcgtggatatcgagtccgaagaggaaggtaatttctacgtgcccccggatatgcgcggggttacgcgggccccggggagacagcgcctgcgttcatcggaccccccctcgcgccacactcaccggcggacccccggaggcgcctgccccgccacccagtttccaccccccatgtccgatagcgaataa

SEQ ID NO: 20 - protein, 96 aa, Herpes Virus
MGLSFSGARPCCCRNNVLITDDGEVVSLTAHDFDVVDIESEEEGNFYVPPDMRGVTRAPGRQRLRSSDPPSRHTHRRTPGGACPATQFPPPMSDSE

SEQ ID NO: 21 - DNA, 801 bp, Herpes Virus
atggatgagtcccgcagacagcgacctgctggtcatgtggcagctaacctcagcccccaaggtgcacgccaacggtccttcaaggattggctcgcatcctacgtacactccaacccccacggggcctccgggcgccccagcggcccctctctccaggacgccgccgtctcccgctcctcccacgggtcccgccaccgatccggcctccgcgagcggcttcgcgcgggactatcccgatggcgaatgagccgctcgtctcatcgccgcgcgtcccccgagacgcccggtacggcggccaaactgaaccgcccgcccctgcgcagatcccaggcggcgttaaccgcacccccctcgtccccctcgcacatcctcaccctcacgcgcatccgcaagctatgcagccccgtgttcgccatcaaccccgccctacactacacgaccctcgagatccccggggcccgaagcttcggggggtctgggggatacggtgacgtccaactgattcgcgaacataagcttgccgttaagaccataaaggaaaaggagtggtttgccgttgagctcatcgcgaccctgttggtcggggagtgcgttctacgcgccggccgcacccacaacatccgcggcttcatcgcgcccctcgggttctcgctgcaacaacgacagatagtgttccccgcgtacgacatggacctcggtaagtatatcggccaactggcgtccctgcgcacaacaaacccctcggtctcgacggccctccaccagtgcttcacggagctggcccgcgccgttgtgttttta

SEQ ID NO: 22 - protein, 267 aa, Herpes Virus
MDESRRQRPAGHVAANLSPQGARQRSFKDWLASYVHSNPHGASGRPSGPSLQDAAVSRSSHGSRHRSGLRERLRAGLSRWRMSRSSHRRASPETPGTAAKLNRPPLRRSQAALTAPPSSPSHILTLTRIRKLCSPVFAINPALHYTTLEIPGARSFGGSGGYGDVQLIREHKLAVKTIKEKEWFAVELIATLLVGECVLRAGRTHNIRGFIAPLGFSLQQRQIVFPAYDMDLGKYIGQLASLRTTNPSVSTALHQCFTELARAVVFL

SEQ ID NO: 23 - DNA, 1557 bp, Herpes Virus
atggatgagtcccgcagacagcgacctgctggtcatgtggcagctaacctcagcccccaaggtgcacgccaacggtccttcaaggattggctcgcatcctacgtacactccaacccccacggggcctccgggcgccccagcggcccctctctccaggacgccgccgtctcccgctcctcccacgggtcccgccaccgatccggcctccgcgagcggcttcgcgcgggactatcccgatggcgaatgagccgctcgtctcatcgccgcgcgtcccccgagacgcccggtacggcggccaaactgaaccgcccgcccctgcgcagatcccaggcggcgttaaccgcacccccctcgtccccctcgcacatcctcaccctcacgcgcatccgcaagctatgcagccccgtgttcgccatcaaccccgccctacactacacgaccctcgagatccccggggcccgaagcttcggggggtctgggggatacggtgacgtccaactgattcgcgaacataagcttgccgttaagaccataaaggaaaaggagtggtttgccgttgagctcatcgcgaccctgttggtcggggagtgcgttctacgcgccggccgcacccacaacatccgcggcttcatcgcgcccctcgggttctcgctgcaacaacgacagatagtgttccccgcgtacgacatggacctcggtaagtatatcggccaactggcgtccctgcgcacaacaaacccctcggtctcgacggccctccaccagtgcttcacggagctggcccgcgccgttgtgtttttaaacaccacctgcgggatcagccacctggatatcaagtgcgccaacatcctcgtcatgctgcggtcggacgccgtctcgctccggcgggccgtcctcgccgactttagcctcgtcaccctcaactccaactccacgatcgcccgggggcagttttgcctccaggagccggacctcaagtccccccggatgtttggcatgcccaccgccctaaccacagccaactttcacaccctggtgggtcacgggtataaccagcccccggagctgttggtgaaataccttaacaacgaacgggccgaatttaccaaccaccgcctgaagcacgacgtcgggttagcggttgacctgtacgccctgggccagacgctgctggagttggtggttagcgtgtacgtcgccccgagcctgggcgtacccgtgacccggtttcccggttaccagtattttaacaaccagctgtcgccggacttcgccctggccctgctcgcctatcgctgcgtgctgcacccagccctgtttgtcaactcggccgagaccaacacccacggcctggcgtatgacgtcccagagggcatccggcgccacctccgcaatcccaagattcggcgcgcgtttacggatcggtgtataaattaccagcacacacacaaggcgatactgtcgtcggtggcgctgcctcccgagcttaagcctctcctggtgctggtgtcccgcctgtgtcacaccaacccgtgcgcgcggcacgcgctgtcgtga

SEQ ID NO: 24 - protein, 518 aa, Herpes Virus
MDESRRQRPAGHVAANLSPQGARQRSFKDWLASYVHSNPHGASGRPSGPSLQDAAVSRSSHGSRHRSGLRERLRAGLSRWRMSRSSHRRASPETPGTAAKLNRPPLRRSQAALTAPPSSPSHILTLTRIRKLCSPVFAINPALHYTTLEIPGARSFGGSGGYGDVQLIREHKLAVKTIKEKEWFAVELIATLLVGECVLRAGRTHNIRGFIAPLGFSLQQRQIVFPAYDMDLGKYIGQLASLRTTNPSVSTALHQCFTELARAVVFLNTTCGISHLDIKCANILVMLRSDAVSLRRAVLADFSLVTLNSNSTIARGQFCLQEPDLKSPRMFGMPTALTTANFHTLVGHGYNQPPELLVKYLNNERAEFTNHRLKHDVGLAVDLYALGQTLLELVVSVYVAPSLGVPVTRFPGYQYFNNQLSPDFALALLAYRCVLHPALFVNSAETNTHGLAYDVPEGIRRHLRNPKIRRAFTDRCINYQHTHKAILSSVALPPELKPLLVLVSRLCHTNPCARHALS

SEQ ID NO: 25 - DNA, 312 bp, Herpes Virus
cagcagtacctggagcgcctcgagaaacagaggcaacttaaggtgggcgcggacgaggcgtcggcgggcctcaccatgggcggcgatgccctacgagtgccctttttagatttcgcgaccgcgacccccaagcgccaccagaccgtggtccctggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggta

SEQ ID NO: 26 - protein, 104 aa, Herpes Virus
QQYLERLEKQRQLKVGADEASAGLTMGGDALRVPFLDFATATPKRHQTVVPGVGTLHDCCEHSPLFSAVARRLLFNSLVPAQLKGRDFGGDHTAKLEFLAPELV

SEQ ID NO: 27 - DNA, 2208 bp, Herpes Virus
atgtttggtcagcagctggcgtccgacgtccagcagtacctggagcgcctcgagaaacagaggcaacttaaggtgggcgcggacgaggcgtcggcgggcctcaccatgggcggcgatgccctacgagtgccctttttagatttcgcgaccgcgacccccaagcgccaccagaccgtggtccctggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaatacgtttcaggccctccaccgctccgaagcctttcgccagctggtgcactttgtgcgggactttgcccagctgctcaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctggtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgcagacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctaaataacctcaccgaaaccgtctcccccaacactgacgtccgtacgtattccggaaaacggaacggcgcctcggatgaccttatggtcgccgtcattatggccatctacctcgcggcccaggccggacctccgcacacattcgctcctatcacacgcgtctcgtga

SEQ ID NO: 28 - protein, 735 aa, Herpes Virus
MFGQQLASDVQQYLERLEKQRQLKVGADEASAGLTMGGDALRVPFLDFATATPKRHQTVVPGVGTLHDCCEHSPLFSAVARRLLFNSLVPAQLKGRDFGGDHTAKLEFLAPELVRAVARLRFKECAPADVVPQRNAYYSVLNTFQALHRSEAFRQLVHFVRDFAQLLKTSFRASSLTETTGPPKKRAKVDVATHGRTYGTLELFQKMILMHATYFLAAVLLGDHAEQVNTFLRLVFEIPLFSDAAVRHFRQRATVFLVPRRHGKTWFLVPLIALSLASFRGIKIGYTAHIRKATEPVFEEIDACLRGWFGSARVDHVKGETISFSFPDGSRSTIVFASSHNTNGIRGQDFNLLFVDEANFIRPDAVQTIMGFLNQANCKIIFVSSTNTGKASTSFLYNLRGAADELLNVVTYICDDHMPRVVTHTNATACSCYILNKPVFITMDGAVRRTADLFLADSFMQEIIGGQARETGDDRPVLTKSAGERFLLYRPSTTTNSGLMAPDLYVYVDPAFTANTRASGTGVAVVGRYRDDYIIFALEHFFLRALTGSAPADIARCVVHSLTQVLALHPGAFRGVRVAVEGNSSQDSAVAIATHVHTEMHRLLASEGADAGSGPELLFYHCEPPGSAVLYPFFLLNKQKTPAFEHFIKKFNSGGVMASQEIVSATVRLQTDPVEYLLEQLNNLTETVSPNTDVRTYSGKRNGASDDLMVAVIMAIYLAAQAGPPHTFAPITRVS

SEQ ID NO: 29 - DNA, 312 bp, Herpes Virus
aacgcactgtggctctcctccgtcgtaacggagtcctgtccctgcgtcgccccgtgtctgtgggccaagatggcccagtgtaccctggcggtccagggggatgctagcctgtgtccgcttctctttggccatcccgtggatacggtcaccctgctgcaggccccccgccgtccttgcatcacggaccgtctgcaagaggtcgtcgggggacggtgcggcgcggacaacatccccccgaccagcgccgggtggcgcctgtgtgtcttctcttcgtacatcagtcgcctatttgctacgagttgccccaccgtt

SEQ ID NO: 30 - protein, 104 aa, Herpes Virus
NALWLSSVVTESCPCVAPCLWAKMAQCTLAVQGDASLCPLLFGHPVDTVTLLQAPRRPCITDRLQEVVGGRCGADNIPPTSAGWRLCVFSSYISRLFATSCPTV

SEQ ID NO: 31 - DNA, 1122 bp, Herpes Virus
atggcgcagctgggaccccggcggcccctggcgccgcctggtcccccggggaccttgccccggccggattcccgggccggagctcgcggcacgcgcgatagagtcgacgacctggggacggacgtcgactctatcgcgcgcattgtcaactccgtctttgtgtggcgcgtcgttcgggcggacgagcggctcaagatctttcggtgtctaacggtcctcaccgagcctctgtgtcaggtggcccttcctaacccagaccccgggcgcgccctcttctgcgagatttttctgtatctgacgcgccccaaggcgctgcggttgcccccgaacaccttctttgccctctttttctttaaccgcgagcgccgctactgcgcgatcgtccacctccggagcgtgacgcaccccctgaccccgctcctgtgcaccctcacgttcgcacgcatacgggcggccacccccccggaggaaacccccgacccaaccaccgaacagctcgcggaggagccagtggtcggcgagctggatggcgcgtatctggtccccgcgaagacccccccggagccgggcgcgtgctgcgccttgggcccgggggcctggtggcacctccccagcggccagatctactgctgggccatggacagcgacctggggtcgctctgtccaccgggaagcagggcccgccatctgggatggctcctggccaggatcaccaaccacccggggggctgcgagtcctgcgccccgccgccccacatcgattccgccaacgcactgtggctctcctccgtcgtaacggagtcctgtccctgcgtcgccccgtgtctgtgggccaagatggcccagtgtaccctggcggtccagggggatgctagcctgtgtccgcttctctttggccatcccgtggatacggtcaccctgctgcaggccccccgccgtccttgcatcacggaccgtctgcaagaggtcgtcgggggacggtgcggcgcggacaacatccccccgaccagcgccgggtggcgcctgtgtgtcttctcttcgtacatcagtcgcctatttgctacgagttgccccaccgttgcccgggccgttgcccgggcctcctcaagcgatcccgaataa

SEQ ID NO: 32 - protein, 373 aa, Herpes Virus
MAQLGPRRPLAPPGPPGTLPRPDSRAGARGTRDRVDDLGTDVDSIARIVNSVFVWRVVRADERLKIFRCLTVLTEPLCQVALPNPDPGRALFCEIFLYLTRPKALRLPPNTFFALFFFNRERRYCAIVHLRSVTHPLTPLLCTLTFARIRAATPPEETPDPTTEQLAEEPVVGELDGAYLVPAKTPPEPGACCALGPGAWWHLPSGQIYCWAMDSDLGSLCPPGSRARHLGWLLARITNHPGGCESCAPPPHIDSANALWLSSVVTESCPCVAPCLWAKMAQCTLAVQGDASLCPLLFGHPVDTVTLLQAPRRPCITDRLQEVVGGRCGADNIPPTSAGWRLCVFSSYISRLFATSCPTVARAVARASSSDPE

SEQ ID NO: 33 - DNA, 1425 bp, Herpes Virus
tggttgggggaggtgaccaggcgcttccccattctcctcgagaacctgatgcgcgccgtcgaggggaccgcccccgacgccttttttcacaccgcgtatgccctggccgtcctggcacacctggggggacggggcggtcgggggcggcgggtcgtcccgctcggcgacgacctcccggcccgctttgccgactccgacggccattacgtttttgactactacagcacaagcggagacacgctgcggcttaacaatcgtccaatcgccgtggcgatggatggtgacgtcagtaaacgcgagcagagtaaatgtcgcttcatggaggccgtcccctccacagccccacgcagggtctgcgagcaatacctgcccggggaaagctacgcctacctctgcctggggtttaatcgccgcctctgtggcatagttgtctttcccggcggctttgcgttcaccattaacatcgcggcctaccttagcctctcggaccccgtcgcgcgggccgctgtccttaggttttgtcgcaaggtgtcgtccgggaacggccggtctcgctagcgggcgccttcccccggccacctcgcccacccactcctccccgcgccgttggcccccgcctctggggtttgccctccccccgcccccggcatggcgcagctgggaccccggcggcccctggcgccgcctggtcccccggggaccttgccccggccggattcccgggccggagctcgcggcacgcgcgatagagtcgacgacctggggacggacgtcgactctatcgcgcgcattgtcaactccgtctttgtgtggcgcgtcgttcgggcggacgagcggctcaagatctttcggtgtctaacggtcctcaccgagcctctgtgtcaggtggcccttcctaacccagaccccgggcgcgccctcttctgcgagatttttctgtatctgacgcgccccaaggcgctgcggttgcccccgaacaccttctttgccctctttttctttaaccgcgagcgccgctactgcgcgatcgtccacctccggagcgtgacgcaccccctgaccccgctcctgtgcaccctcacgttcgcacgcatacgggcggccacccccccggaggaaacccccgacccaaccaccgaacagctcgcggaggagccagtggtcggcgagctggatggcgcgtatctggtccccgcgaagacccccccggagccgggcgcgtgctgcgccttgggcccgggggcctggtggcacctccccagcggccagatctactgctgggccatggacagcgacctggggtcgctctgtccaccgggaagcagggcccgccatctgggatggctcctggccaggatcaccaaccacccggggggctgcgagtcctgcgccccgccgccccacatcgattccgccaacgcactg

SEQ ID NO: 34 - protein, 185 aa, Herpes Virus
WLGEVTRRFPILLENLMRAVEGTAPDAFFHTAYALAVLAHLGGRGGRGRRVVPLGDDLPARFADSDGHYVFDYYSTSGDTLRLNNRPIAVAMDGDVSKREQSKCRFMEAVPSTAPRRVCEQYLPGESYAYLCLGFNRRLCGIVVFPGGFAFTINIAAYLSLSDPVARAAVLRFCRKVSSGNGRSR

SEQ ID NO: 35 - DNA, 1068 bp, Herpes Virus
gccaacgaggtccagacgatctcggccacggcccgggtcggccctcggtctttggttcacgtcatcatatccagcgagtgcctggcggccgcgggaatccctctggccgccctgatgcgcggccgccccggactcgggacggccgcaaacttccaggtcgaaatccagactcgggctcatgccaccggcgactgtaccccgtggtgcacggcgtttgccgcctacgtgcccgcggatgcggtgggggagcttctggcccccgtcgtgccggcacaccctggcctccttccgcgtgcgtccagcgccggggggttgttcgtctccctgcccgtggtgtgtgacgcgcagggcgtctatgacccgtacgccgtggcggcgctgcgccttgcgtggggctcgggggcgagctgtgcccgcgtgattctgtttagttacgacgagctcgtcccccccaacacgcgctacgcggccgacagcacgcgcatcatgcgcgtctgtcggcatttgtgccgctacgtcgctctgcttggcgccgccgccccgccggccgcgaaggaggctgcggcccacctgtccatgggtctgggggaaagcgcgtccccgcgtccgcagcccttggcccggccccacgcgggggcgcccgcagacccgcccatcgtcggggcgtccgacccccccatctccccggaggagcagctgacggcccccggcggcgacacgaccgcggcccaggacgtgtccatcgcacaggagaacgaggagatcctcgcgttggttcagcgggcagtgcaggacgtcacccgccgccacccggtccgagcgcggaccgggcgtgcggcctgtggcgttgcatcggggctacgccagggcgccctggttcaccaggccgtcagcgggggcgccatgggggcggctgacgcagatgcggtgctggcgggtctggagccccccggcgggggccgctttgtggccccagcgccccacgggcccgggggcgaggacatcctgaacgacgttctaacccttacccctggtaccgcaaagccgcggtcgctggtcgagtggttg

SEQ ID NO: 36 - protein, 356 aa, Herpes Virus
ANEVQTISATARVGPRSLVHVIISSECLAAAGIPLAALMRGRPGLGTAANFQVEIQTRAHATGDCTPWCTAFAAYVPADAVGELLAPVVPAHPGLLPRASSAGGLFVSLPVVCDAQGVYDPYAVAALRLAWGSGASCARVILFSYDELVPPNTRYAADSTRIMRVCRHLCRYVALLGAAAPPAAKEAAAHLSMGLGESASPRPQPLARPHAGAPADPPIVGASDPPISPEEQLTAPGGDTTAAQDVSIAQENEEILALVQRAVQDVTRRHPVRARTGRAACGVASGLRQGALVHQAVSGGAMGAADADAVLAGLEPPGGGRFVAPAPHGPGGEDILNDVLTLTPGTAKPRSLVEWL

SEQ ID NO: 37 - DNA, 1056 bp, Herpes Virus
gttctaacccttacccctggtaccgcaaagccgcggtcgctggtcgagtggttggatcgcggatgggaagccctggccggcggcgaccggccggactggctgtggagccgtcgttctatctccgtggtcctgcgccaccactacggaaccaagcagcgcttcgtcgtcgtctcctacgagaactccgtggcgtggggcgggcgacgcgcccgccctccgctgctgtcctcggcgctggccacggccctgaccgaggcctgcgccgcagaacgcgtcgtgcgcccccaccagctgtctcccgctgggcaggcggagctgctgctacgctttcccgcgctcgaggtgcccctgcgccacccgcgccccgtcctgccgccctttgacatcgccgccgaggtcgcctttaccgcgcgcatacatctggcgtgcctccgggccctgggccaggccatccgggccgcgcttcagggcggcccgcgaatctcacagcgcctgcgctatgactttggccccgaccaacgcgcgtggttgggggaggtgaccaggcgcttccccattctcctcgagaacctgatgcgcgccgtcgaggggaccgcccccgacgccttttttcacaccgcgtatgccctggccgtcctggcacacctggggggacggggcggtcgggggcggcgggtcgtcccgctcggcgacgacctcccggcccgctttgccgactccgacggccattacgtttttgactactacagcacaagcggagacacgctgcggcttaacaatcgtccaatcgccgtggcgatggatggtgacgtcagtaaacgcgagcagagtaaatgtcgcttcatggaggccgtcccctccacagccccacgcagggtctgcgagcaatacctgcccggggaaagctacgcctacctctgcctggggtttaatcgccgcctctgtggcatagttgtctttcccggcggctttgcgttcaccattaacatcgcggcctaccttagcctctcggaccccgtcgcgcgggccgctgtccttaggttttgtcgcaaggtg

SEQ ID NO: 38 - protein, 352 aa, Herpes Virus
VLTLTPGTAKPRSLVEWLDRGWEALAGGDRPDWLWSRRSISVVLRHHYGTKQRFVVVSYENSVAWGGRRARPPLLSSALATALTEACAAERVVRPHQLSPAGQAELLLRFPALEVPLRHPRPVLPPFDIAAEVAFTARIHLACLRALGQAIRAALQGGPRISQRLRYDFGPDQRAWLGEVTRRFPILLENLMRAVEGTAPDAFFHTAYALAVLAHLGGRGGRGRRVVPLGDDLPARFADSDGHYVFDYYSTSGDTLRLNNRPIAVAMDGDVSKREQSKCRFMEAVPSTAPRRVCEQYLPGESYAYLCLGFNRRLCGIVVFPGGFAFTINIAAYLSLSDPVARAAVLRFCRKV

SEQ ID NO: 39 - DNA, 2112 bp, Herpes Virus
atgaacgcgcacttggccaacgaggtccagacgatctcggccacggcccgggtcggccctcggtctttggttcacgtcatcatatccagcgagtgcctggcggccgcgggaatccctctggccgccctgatgcgcggccgccccggactcgggacggccgcaaacttccaggtcgaaatccagactcgggctcatgccaccggcgactgtaccccgtggtgcacggcgtttgccgcctacgtgcccgcggatgcggtgggggagcttctggcccccgtcgtgccggcacaccctggcctccttccgcgtgcgtccagcgccggggggttgttcgtctccctgcccgtggtgtgtgacgcgcagggcgtctatgacccgtacgccgtggcggcgctgcgccttgcgtggggctcgggggcgagctgtgcccgcgtgattctgtttagttacgacgagctcgtcccccccaacacgcgctacgcggccgacagcacgcgcatcatgcgcgtctgtcggcatttgtgccgctacgtcgctctgcttggcgccgccgccccgccggccgcgaaggaggctgcggcccacctgtccatgggtctgggggaaagcgcgtccccgcgtccgcagcccttggcccggccccacgcgggggcgcccgcagacccgcccatcgtcggggcgtccgacccccccatctccccggaggagcagctgacggcccccggcggcgacacgaccgcggcccaggacgtgtccatcgcacaggagaacgaggagatcctcgcgttggttcagcgggcagtgcaggacgtcacccgccgccacccggtccgagcgcggaccgggcgtgcggcctgtggcgttgcatcggggctacgccagggcgccctggttcaccaggccgtcagcgggggcgccatgggggcggctgacgcagatgcggtgctggcgggtctggagccccccggcgggggccgctttgtggccccagcgccccacgggcccgggggcgaggacatcctgaacgacgttctaacccttacccctggtaccgcaaagccgcggtcgctggtcgagtggttggatcgcggatgggaagccctggccggcggcgaccggccggactggctgtggagccgtcgttctatctccgtggtcctgcgccaccactacggaaccaagcagcgcttcgtcgtcgtctcctacgagaactccgtggcgtggggcgggcgacgcgcccgccctccgctgctgtcctcggcgctggccacggccctgaccgaggcctgcgccgcagaacgcgtcgtgcgcccccaccagctgtctcccgctgggcaggcggagctgctgctacgctttcccgcgctcgaggtgcccctgcgccacccgcgccccgtcctgccgccctttgacatcgccgccgaggtcgcctttaccgcgcgcatacatctggcgtgcctccgggccctgggccaggccatccgggccgcgcttcagggcggcccgcgaatctcacagcgcctgcgctatgactttggccccgaccaacgcgcgtggttgggggaggtgaccaggcgcttccccattctcctcgagaacctgatgcgcgccgtcgaggggaccgcccccgacgccttttttcacaccgcgtatgccctggccgtcctggcacacctggggggacggggcggtcgggggcggcgggtcgtcccgctcggcgacgacctcccggcccgctttgccgactccgacggccattacgtttttgactactacagcacaagcggagacacgctgcggcttaacaatcgtccaatcgccgtggcgatggatggtgacgtcagtaaacgcgagcagagtaaatgtcgcttcatggaggccgtcccctccacagccccacgcagggtctgcgagcaatacctgcccggggaaagctacgcctacctctgcctggggtttaatcgccgcctctgtggcatagttgtctttcccggcggctttgcgttcaccattaacatcgcggcctaccttagcctctcggaccccgtcgcgcgggccgctgtccttaggttttgtcgcaaggtgtcgtccgggaacggccggtctcgctag

SEQ ID NO: 40 - protein, 703 aa, Herpes Virus
MNAHLANEVQTISATARVGPRSLVHVIISSECLAAAGIPLAALMRGRPGLGTAANFQVEIQTRAHATGDCTPWCTAFAAYVPADAVGELLAPVVPAHPGLLPRASSAGGLFVSLPVVCDAQGVYDPYAVAALRLAWGSGASCARVILFSYDELVPPNTRYAADSTRIMRVCRHLCRYVALLGAAAPPAAKEAAAHLSMGLGESASPRPQPLARPHAGAPADPPIVGASDPPISPEEQLTAPGGDTTAAQDVSIAQENEEILALVQRAVQDVTRRHPVRARTGRAACGVASGLRQGALVHQAVSGGAMGAADADAVLAGLEPPGGGRFVAPAPHGPGGEDILNDVLTLTPGTAKPRSLVEWLDRGWEALAGGDRPDWLWSRRSISVVLRHHYGTKQRFVVVSYENSVAWGGRRARPPLLSSALATALTEACAAERVVRPHQLSPAGQAELLLRFPALEVPLRHPRPVLPPFDIAAEVAFTARIHLACLRALGQAIRAALQGGPRISQRLRYDFGPDQRAWLGEVTRRFPILLENLMRAVEGTAPDAFFHTAYALAVLAHLGGRGGRGRRVVPLGDDLPARFADSDGHYVFDYYSTSGDTLRLNNRPIAVAMDGDVSKREQSKCRFMEAVPSTAPRRVCEQYLPGESYAYLCLGFNRRLCGIVVFPGGFAFTINIAAYLSLSDPVARAAVLRFCRKVSSGNGRSR

SEQ ID NO: 41 - DNA, 942 bp, Herpes Virus
gacggctttgaaactgacatcgcgataccctcgggcatctcgcgccccgatgcggcggcgctgcagcgctgcgaagggcgggtggtattcctgccgaccatccgccggcaactgacgctggccgacgtggcgcacgaatccttcgtctccggaggcgtcagtcccgacacgttggggttgttgctggcgtaccgaaggcgcttccccgcggtcatcacccgggtgcttcccacgcgaatcgtcgcctgccccctggacgtgggcctcacccacgccggcaccgttaaccttcgcaacacctcccccgtagatctctgtaacggggaccccatcagcctcgtcccgcccgtgttcgagggccaagcgacggacgtgcgcctggattcgctggacctcacgttgcggtttcccgttccgcttccatcgcccctggcgcgcgaaatcgtggcgcggctcgtggccaggggcatccgggacctgaaccccagccccagaaaccccggagggctgccagacctcaacgtgctgtactacaacgggagtcgcctctcgctgctggcggacgtccaacaactcggtcccgtaaacgccgagctgcgatcgctggtccttaacatggtttactcgatcacggagggaaccaccatcatccttacgctaatcccccggctctttgcgctaagtgcccaggacgggtacgtgaacgctctactgcagatgcagagtgtcacgcgggaggccgcccagctcattcaccccgaagccccggccctgatgcaggatggagagcgaaggctgccgctttacgaggcgctcgtcgcctggctgacccacgcgggccaactaggagacaccttggccctggctcccgtggttcgggtgtgcacctttgacggcgcggccgttgtgcggtccggagacatggcccccgttatacgctat

SEQ ID NO: 42 - protein, 314 aa, Herpes Virus
DGFETDIAIPSGISRPDAAALQRCEGRVVFLPTIRRQLTLADVAHESFVSGGVSPDTLGLLLAYRRRFPAVITRVLPTRIVACPLDVGLTHAGTVNLRNTSPVDLCNGDPISLVPPVFEGQATDVRLDSLDLTLRFPVPLPSPLAREIVARLVARGIRDLNPSPRNPGGLPDLNVLYYNGSRLSLLADVQQLGPVNAELRSLVLNMVYSITEGTTIILTLIPRLFALSAQDGYVNALLQMQSVTREAAQLIHPEAPALMQDGERRLPLYEALVAWLTHAGQLGDTLALAPVVRVCTFDGAAVVRSGDMAPVIRY

SEQ ID NO: 43 - DNA, 957 bp, Herpes Virus
atgctggcggacggctttgaaactgacatcgcgataccctcgggcatctcgcgccccgatgcggcggcgctgcagcgctgcgaagggcgggtggtattcctgccgaccatccgccggcaactgacgctggccgacgtggcgcacgaatccttcgtctccggaggcgtcagtcccgacacgttggggttgttgctggcgtaccgaaggcgcttccccgcggtcatcacccgggtgcttcccacgcgaatcgtcgcctgccccctggacgtgggcctcacccacgccggcaccgttaaccttcgcaacacctcccccgtagatctctgtaacggggaccccatcagcctcgtcccgcccgtgttcgagggccaagcgacggacgtgcgcctggattcgctggacctcacgttgcggtttcccgttccgcttccatcgcccctggcgcgcgaaatcgtggcgcggctcgtggccaggggcatccgggacctgaaccccagccccagaaaccccggagggctgccagacctcaacgtgctgtactacaacgggagtcgcctctcgctgctggcggacgtccaacaactcggtcccgtaaacgccgagctgcgatcgctggtccttaacatggtttactcgatcacggagggaaccaccatcatccttacgctaatcccccggctctttgcgctaagtgcccaggacgggtacgtgaacgctctactgcagatgcagagtgtcacgcgggaggccgcccagctcattcaccccgaagccccggccctgatgcaggatggagagcgaaggctgccgctttacgaggcgctcgtcgcctggctgacccacgcgggccaactaggagacaccttggccctggctcccgtggttcgggtgtgcacctttgacggcgcggccgttgtgcggtccggagacatggcccccgttatacgctatccctaa

SEQ ID NO: 44 - protein, 318 aa, Herpes Virus
MLADGFETDIAIPSGISRPDAAALQRCEGRVVFLPTIRRQLTLADVAHESFVSGGVSPDTLGLLLAYRRRFPAVITRVLPTRIVACPLDVGLTHAGTVNLRNTSPVDLCNGDPISLVPPVFEGQATDVRLDSLDLTLRFPVPLPSPLAREIVARLVARGIRDLNPSPRNPGGLPDLNVLYYNGSRLSLLADVQQLGPVNAELRSLVLNMVYSITEGTTIILTLIPRLFALSAQDGYVNALLQMQSVTREAAQLIHPEAPALMQDGERRLPLYEALVAWLTHAGQLGDTLALAPVVRVCTFDGAAVVRSGDMAPVIRYP

SEQ ID NO: 45 - DNA, 798 bp, Herpes Virus
cctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgt

SEQ ID NO: 46 - protein, 266 aa, Herpes Virus
PPPNNTDSSSLVPGAQDSAPPGPTLRELWWVFYAADRALEEPRADSGLTREEVRAVRGFREQAWKLFGSAGAPRAFIGAALGLSPLQKLAVYYYIIHRERRLSPFPALVRLVGRYTQRHGLYVPRPDDPVLADAINGLFRDALAAGTTAEQLLMFDLLPPKDVPVGSDVQADSTALLRFIESQRLAVPGGVISPEHVAYLGAFLSVLYAGRGRMSAATHTARLTGVTSLVLAVGDVDRLSAFDRGAAGAASRTRAAGYLDVLLTVR

SEQ ID NO: 47 - DNA, 1608 bp, Herpes Virus
atggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaa

SEQ ID NO: 48 - protein, 535 aa, Herpes Virus
MELSYATTMHYRDVVFYVTTDRNRAYFVCGGCVYSVGRPCASQPGEIAKFGLVVRGTGPDDRVVANYVRSELRQRGLQDVRPIGEDEVFLDSVCLLNPNVSSELDVINTNDVEVLDECLAEYCTSLRTSPGVLISGLRVRAQDRIIELFEHPTIVNVSSHFVYTPSPYVFALAQAHLPRLPSSLEALVSGLFDGIPAPRQPLDAHNPRTDVVITGRRAPRPIAGSGAGSGGAGAKRATVSEFVQVKHIDRVGPAGVSPAPPPNNTDSSSLVPGAQDSAPPGPTLRELWWVFYAADRALEEPRADSGLTREEVRAVRGFREQAWKLFGSAGAPRAFIGAALGLSPLQKLAVYYYIIHRERRLSPFPALVRLVGRYTQRHGLYVPRPDDPVLADAINGLFRDALAAGTTAEQLLMFDLLPPKDVPVGSDVQADSTALLRFIESQRLAVPGGVISPEHVAYLGAFLSVLYAGRGRMSAATHTARLTGVTSLVLAVGDVDRLSAFDRGAAGAASRTRAAGYLDVLLTVRLARSQHGQSV

SEQ ID NO: 49 - DNA, 834 bp, Herpes Virus
gattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcaggcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttc

SEQ ID NO: 50 - protein, 278 aa, Herpes Virus
DSRNFITPEFPRDFWMSPVFNLPRETAAEQVVVLQAQRTAAAAALENAAMQAAELPVDIERRLRPIERNVHEIAGALEALETAAAAAEEADAARGDEPAGGGDGGAPPGLAVAEMEVQIVRNDPPLRYDTNLPVDLLHMVYAGRGATGSSGVVFGTWYRTIQDRTITDFPLTTRSADFRDGRMSKTFMTALVLSLQACGRLYVGQRHYSAFECAVLCLYLLYRNTHGAADDSDRAPVTFGDLLGRLPRYLACLAAVIGTEGGRPQYRYRDDKLPKTQF

SEQ ID NO: 51 - DNA, 1743 bp, Herpes Virus
atggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcaggcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagtacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccctgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttag

SEQ ID NO: 52 - protein, 580 aa, Herpes Virus
MDPYCPFDALDVWEHRRFIVADSRNFITPEFPRDFWMSPVFNLPRETAAEQVVVLQAQRTAAAAALENAAMQAAELPVDIERRLRPIERNVHEIAGALEALETAAAAAEEADAARGDEPAGGGDGGAPPGLAVAEMEVQIVRNDPPLRYDTNLPVDLLHMVYAGRGATGSSGVVFGTWYRTIQDRTITDFPLTTRSADFRDGRMSKTFMTALVLSLQACGRLYVGQRHYSAFECAVLCLYLLYRNTHGAADDSDRAPVTFGDLLGRLPRYLACLAAVIGTEGGRPQYRYRDDKLPKTQFAAGGGRYEHGALASHIVIATLMHHGVLPAAPGDVPRDASTHVNPDGVAHHDDINRAAAAFLSRGHNLFLWEDQTLLRATANTITALGVIQRLLANGNVYADRLNNRLQLGMLIPGAVPSEAIARGASGSDSGAIKSGDNNLEALCANYVLPLYRADPAVELTQLFPGLAALCLDAQAGRPVGSTRRVVDMSSGARQAALVRLTALELINRTRTNPTPVGEVIHAHDALAIQYEQGLGLLAQQARIGLGSNTKRFSAFNVSSDYDMLYFLCLGFIPQYLSAV

SEQ ID NO: 53 - DNA, 683 bp, Herpes Virus
gtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtt

SEQ ID NO: 54 - protein, 227 aa, Herpes Virus
VYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQ

SEQ ID NO: 55 - DNA, 2715 bp, Herpes Virus
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacagaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 56 - protein, 904 aa, Herpes Virus
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEESAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 57 - DNA, 1814 bp, Herpes Virus
ctgctagagtacgcgtggcgcgagggcgagcggctcctgggcagcctggagacgttcgcgaccgcgggagacgtcgcggcgtttttcacggagaccatgggcctggcccgaccctgtccgtatcaccaacgggtcaggctggatacgtatggcgggaccgtccatatggagctgtgtttcctgcacgacgtcgagaactttctaaagcagctaaactactgccacctcatcaccccctcgcgcggcgccaccgccgcgctggagcgcgttcgggagtttatggtgggggcggtggggtcgggccttatcgtccccccggagcttagcgacccgtcccacccctgcgcggtctgtttcgaggaactgtgtgtgacggcgaaccagggggcgacgatcgcccgccgcctggcggaccgtatctgtaaccacgtcacccagcaggcgcaggtgcggctggacgccaacgagctgcggcggtacctgccccacgccgccgggctgtcggacgccgaccgcgcgcgggcgctctccgtgttggaccatgcgctggcccggaccgcggggggcgacgggcagccccacccgtcgcccgagaacgactcggtccgcaaggaggccgacgccctgctggaggcgcacgacgtgtttcaggccaccacgcccggcctgtacgccatcagcgaattgcgattctggctcgcgtccggcgaccgcgccggccagaccaccatggacgcgtttgccagcaacctgaccgcgctggcgcggcgcgagttgcagcaggagaccgccgcggtggccgtggaactggcgctgttcgggcggcgggcggagcatttcgatcgcgcgttcgggagccacctggcggcgctggatatggtggacgccctaataatcggcggtcaggccacgtcacccgacgatcagatcgaggcgctcatccgcgcgtgctacgaccaccacctgacgacgccgctcttgcggcgcctcgtcagccccgaacagtgcgacgaggaggcgctgcgtcgcgtgctggcgcgcatgggggcggggggcgcggcggacgcgcccaagggcggcgcgggccccgacgacgacggggaccgtgtcgccgtagaggaaggggcacgggggttgggagctcccgggggcgggggcgaggacgaagaccgtcgccgcgggcccggggggcaggggcccgagacgtggggggacatcgccacgcaagcggccgcggacgtgcgggagcgacggcggctgtacgcggaccgcctgacgaagcggtcgttggccagcctggggcgctgcgtccgcgagcagcgcggggagctcgagaagatgctgcgggtcagcgtccacggcgaggtgctgcccgcgacgttcgccgcggtcgccaacggctttgcggcgcgcgcgcgcttctgcgccctgacggcgggcgcgggcacggtcatcgacaaccgctcggcgccgggcgtgttcgacgcgcaccggttcatgcgagcgtctctcctgcgacaccaggtggacccggccctgctccccagcatcacccatcgcttcttcgagctcgtcaacgggcccctctttgatcactccacccacagcttcgcccagccccccaacaccgcgctgtattacagcgtcgagaacgtggggctcctgccgcacctgaaggaggagctcgcccggttcatcatgggggcggggggctcgggtgctgattgggccgtcagcgaatttcagaggttttactgttttgacggcatttccggaataacgcccactcagcgcgccgcctggcgatatattcg

SEQ ID NO: 58 - protein, 604 aa, Herpes Virus
LLEYAWREGERLLGSLETFATAGDVAAFFTETMGLARPCPYHQRVRLDTYGGTVHMELCFLHDVENFLKQLNYCHLITPSRGATAALERVREFMVGAVGSGLIVPPELSDPSHPCAVCFEELCVTANQGATIARRLADRICNHVTQQAQVRLDANELRRYLPHAAGLSDADRARALSVLDHALARTAGGDGQPHPSPENDSVRKEADALLEAHDVFQATTPGLYAISELRFWLASGDRAGQTTMDAFASNLTALARRELQQETAAVAVELALFGRRAEHFDRAFGSHLAALDMVDALIIGGQATSPDDQIEALIRACYDHHLTTPLLRRLVSPEQCDEEALRRVLARMGAGGAADAPKGGAGPDDDGDRVAVEEGARGLGAPGGGGEDEDRRRGPGGQGPETWGDIATQAAADVRERRRLYADRLTKRSLASLGRCVREQRGELEKMLRVSVHGEVLPATFAAVANGFAARARFCALTAGAGTVIDNRSAPGVFDAHRFMRASLLRHQVDPALLPSITHRFFELVNGPLFDHSTHSFAQPPNTALYYSVENVGLLPHLKEELARFIMGAGGSGADWAVSEFQRFYCFDGISGITPTQRAAWRYI

SEQ ID NO: 59 - DNA, 1068 bp, Herpes Virus
tatgtgtttcagatagagctgctccggcggtgcgacccccacatcggacgggggaagctcccccaactgaagctgaacgcgcttcaggtgcgggcgctgcggcgtcgtctgaggccgggcctggaggcccaggccggggcctttctcaccccgctgtcggtcaccctggagttgctgctagagtacgcgtggcgcgagggcgagcggctcctgggcagcctggagacgttcgcgaccgcgggagacgtcgcggcgtttttcacggagaccatgggcctggcccgaccctgtccgtatcaccaacgggtcaggctggatacgtatggcgggaccgtccatatggagctgtgtttcctgcacgacgtcgagaactttctaaagcagctaaactactgccacctcatcaccccctcgcgcggcgccaccgccgcgctggagcgcgttcgggagtttatggtgggggcggtggggtcgggccttatcgtccccccggagcttagcgacccgtcccacccctgcgcggtctgtttcgaggaactgtgtgtgacggcgaaccagggggcgacgatcgcccgccgcctggcggaccgtatctgtaaccacgtcacccagcaggcgcaggtgcggctggacgccaacgagctgcggcggtacctgccccacgccgccgggctgtcggacgccgaccgcgcgcgggcgctctccgtgttggaccatgcgctggcccggaccgcggggggcgacgggcagccccacccgtcgcccgagaacgactcggtccgcaaggaggccgacgccctgctggaggcgcacgacgtgtttcaggccaccacgcccggcctgtacgccatcagcgaattgcgattctggctcgcgtccggcgaccgcgccggccagaccaccatggacgcgtttgccagcaacctgaccgcgctggcgcggcgcgagttgcagcaggagaccgccgcggtggccgtggaactggcgctgttcgggcggcgggcggagcatttcgatcgcgcgttcgggagccacctggcggcgctggatatggtggacgccctaata

SEQ ID NO: 60 - protein, 356 aa, Herpes Virus
YVFQIELLRRCDPHIGRGKLPQLKLNALQVRALRRRLRPGLEAQAGAFLTPLSVTLELLLEYAWREGERLLGSLETFATAGDVAAFFTETMGLARPCPYHQRVRLDTYGGTVHMELCFLHDVENFLKQLNYCHLITPSRGATAALERVREFMVGAVGSGLIVPPELSDPSHPCAVCFEELCVTANQGATIARRLADRICNHVTQQAQVRLDANELRRYLPHAAGLSDADRARALSVLDHALARTAGGDGQPHPSPENDSVRKEADALLEAHDVFQATTPGLYAISELRFWLASGDRAGQTTMDAFASNLTALARRELQQETAAVAVELALFGRRAEHFDRAFGSHLAALDMVDALI

SEQ ID NO: 61 - DNA, 369 bp, Herpes Virus
gagaagatgctgcgggtcagcgtccacggcgaggtgctgcccgcgacgttcgccgcggtcgccaacggctttgcggcgcgcgcgcgcttctgcgccctgacggcgggcgcgggcacggtcatcgacaaccgctcggcgccgggcgtgttcgacgcgcaccggttcatgcgagcgtctctcctgcgacaccaggtggacccggccctgctccccagcatcacccatcgcttcttcgagctcgtcaacgggcccctctttgatcactccacccacagcttcgcccagccccccaacaccgcgctgtattacagcgtcgagaacgtggggctcctgccgcacctgaaggaggagctcgcccggttcatcatg

SEQ ID NO: 62 - protein, 123 aa, Herpes Virus
EKMLRVSVHGEVLPATFAAVANGFAARARFCALTAGAGTVIDNRSAPGVFDAHRFMRASLLRHQVDPALLPSITHRFFELVNGPLFDHSTHSFAQPPNTALYYSVENVGLLPHLKEELARFIM

SEQ ID NO: 63 - DNA, 2358 bp, Herpes Virus
atggccgccccggtgtccgagcccaccgtggcccgtcaaaagttgttagccctgctcgggcaggtgcagacctatgtgtttcagatagagctgctccggcggtgcgacccccacatcggacgggggaagctcccccaactgaagctgaacgcgcttcaggtgcgggcgctgcggcgtcgtctgaggccgggcctggaggcccaggccggggcctttctcaccccgctgtcggtcaccctggagttgctgctagagtacgcgtggcgcgagggcgagcggctcctgggcagcctggagacgttcgcgaccgcgggagacgtcgcggcgtttttcacggagaccatgggcctggcccgaccctgtccgtatcaccaacgggtcaggctggatacgtatggcgggaccgtccatatggagctgtgtttcctgcacgacgtcgagaactttctaaagcagctaaactactgccacctcatcaccccctcgcgcggcgccaccgccgcgctggagcgcgttcgggagtttatggtgggggcggtggggtcgggccttatcgtccccccggagcttagcgacccgtcccacccctgcgcggtctgtttcgaggaactgtgtgtgacggcgaaccagggggcgacgatcgcccgccgcctggcggaccgtatctgtaaccacgtcacccagcaggcgcaggtgcggctggacgccaacgagctgcggcggtacctgccccacgccgccgggctgtcggacgccgaccgcgcgcgggcgctctccgtgttggaccatgcgctggcccggaccgcggggggcgacgggcagccccacccgtcgcccgagaacgactcggtccgcaaggaggccgacgccctgctggaggcgcacgacgtgtttcaggccaccacgcccggcctgtacgccatcagcgaattgcgattctggctcgcgtccggcgaccgcgccggccagaccaccatggacgcgtttgccagcaacctgaccgcgctggcgcggcgcgagttgcagcaggagaccgccgcggtggccgtggaactggcgctgttcgggcggcgggcggagcatttcgatcgcgcgttcgggagccacctggcggcgctggatatggtggacgccctaataatcggcggtcaggccacgtcacccgacgatcagatcgaggcgctcatccgcgcgtgctacgaccaccacctgacgacgccgctcttgcggcgcctcgtcagccccgaacagtgcgacgaggaggcgctgcgtcgcgtgctggcgcgcatgggggcggggggcgcggcggacgcgcccaagggcggcgcgggccccgacgacgacggggaccgtgtcgccgtagaggaaggggcacgggggttgggagctcccgggggcgggggcgaggacgaagaccgtcgccgcgggcccggggggcaggggcccgagacgtggggggacatcgccacgcaagcggccgcggacgtgcgggagcgacggcggctgtacgcggaccgcctgacgaagcggtcgttggccagcctggggcgctgcgtccgcgagcagcgcggggagctcgagaagatgctgcgggtcagcgtccacggcgaggtgctgcccgcgacgttcgccgcggtcgccaacggctttgcggcgcgcgcgcgcttctgcgccctgacggcgggcgcgggcacggtcatcgacaaccgctcggcgccgggcgtgttcgacgcgcaccggttcatgcgagcgtctctcctgcgacaccaggtggacccggccctgctccccagcatcacccatcgcttcttcgagctcgtcaacgggcccctctttgatcactccacccacagcttcgcccagccccccaacaccgcgctgtattacagcgtcgagaacgtggggctcctgccgcacctgaaggaggagctcgcccggttcatcatgggggcggggggctcgggtgctgattgggccgtcagcgaatttcagaggttttactgttttgacggcatttccggaataacgcccactcagcgcgccgcctggcgatatattcgcgagctgattatcgccaccacactctttgcctcggtctaccggtgcggggagctcgagttgcgccgcccggactgcagccgcccgacctccgaaggtcgttaccgttacccgcccggcgtatatctcacgtacgactccgactgtccgctggtggccatcgtcgagagcgcccccgacggctgtatcggcccccggtcggtcgtggtctacgaccgagacgttttctcgatcctctactcggtcctccagcacctcgcccccaggctacctgacggggggcacgacgggcccccgtag

SEQ ID NO: 64 - protein, 785 aa, Herpes Virus
MAAPVSEPTVARQKLLALLGQVQTYVFQIELLRRCDPHIGRGKLPQLKLNALQVRALRRRLRPGLEAQAGAFLTPLSVTLELLLEYAWREGERLLGSLETFATAGDVAAFFTETMGLARPCPYHQRVRLDTYGGTVHMELCFLHDVENFLKQLNYCHLITPSRGATAALERVREFMVGAVGSGLIVPPELSDPSHPCAVCFEELCVTANQGATIARRLADRICNHVTQQAQVRLDANELRRYLPHAAGLSDADRARALSVLDHALARTAGGDGQPHPSPENDSVRKEADALLEAHDVFQATTPGLYAISELRFWLASGDRAGQTTMDAFASNLTALARRELQQETAAVAVELALFGRRAEHFDRAFGSHLAALDMVDALIIGGQATSPDDQIEALIRACYDHHLTTPLLRRLVSPEQCDEEALRRVLARMGAGGAADAPKGGAGPDDDGDRVAVEEGARGLGAPGGGGEDEDRRRGPGGQGPETWGDIATQAAADVRERRRLYADRLTKRSLASLGRCVREQRGELEKMLRVSVHGEVLPATFAAVANGFAARARFCALTAGAGTVIDNRSAPGVFDAHRFMRASLLRHQVDPALLPSITHRFFELVNGPLFDHSTHSFAQPPNTALYYSVENVGLLPHLKEELARFIMGAGGSGADWAVSEFQRFYCFDGISGITPTQRAAWRYIRELIIATTLFASVYRCGELELRRPDCSRPTSEGRYRYPPGVYLTYDSDCPLVAIVESAPDGCIGPRSVVVYDRDVFSILYSVLQHLAPRLPDGGHDGPP

SEQ ID NO: 65 - DNA, 514 bp, Herpes Virus
tacctggcgcgcgccgcgggactcgtgggggccatggtatttagcaccaactcggccctccatctcaccgaggtggacgacgccggcccggcggacccaaaggaccacagcaaaccctccttttaccgcttcttcctcgtgcccgggacccacgtggcggccaacccacaggtggaccgcgagggacacgtggtgcccgggttcgagggtcggcccaccgcgcccctcgtcggcggaacccaggaatttgccggcgagcacctggccatgctgtgtgggttttccccggcgctgctggccaagatgctgttttacctggagcgctgcgacggcggcgtgatcgtcgggcgccaggagatggacgtgtttcgatacgtcgcggactccaaccagaccgacgtgccctgtaacctatgcaccttcgacacgcgccacgcctgcgtacacacgacgctcatgcgcctccgggcgcgccatccaaagttcgccagcgccgcccgcggagccatcggcg

SEQ ID NO: 66 - protein, 171 aa, Herpes Virus
YLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIG

SEQ ID NO: 67 - DNA, 3591 bp, Herpes Virus
atggagacaaagcccaagacggcaaccaccatcaaggtcccccccgggcccctgggatacgtgtacgctcgcgcgtgtccgtccgaaggcatcgagcttctggcgttactgtcggcacgcagcggcgattccgacgtcgccgtggcgcccctggtcgtgggcctgaccgtggagagcggctttgaggccaacgtggccgtggtcgtgggttctcgcacgacggggctcgggggtaccgcggtgtccctgaaactgacgccctcgcactacagctcgtccgtgtacgtctttcacggcggccggcacctggaccccagcacccaggccccgaacctgacgcgactttgcgagcgggcacgccgccattttggcttttcggactacaccccccggcccggcgacctcaaacacgagacgacgggggaggcgctgtgtgagcgcctcggcctggacccggaccgcgccctcctgtatctggtcgttaccgagggcttcaaggaggccgtgtgcatcaacaacacctttctgcacctgggaggctcggacaaggtaaccataggcggggcggaggtgcaccgcatacccgtgtacccgttgcagctgttcatgccggattttagccgtgtcatcgcagagccgttcaacgccaaccaccgatcgatcggggagaaatttacctacccgcttccgttttttaaccgccccctcaaccgcctcctgttcgaggcggtcgtgggacccgccgccgtggcactgcgatgccgaaacgtggacgccgtggcccgcgcggccgcccacctggcgtttgacgaaaaccacgagggcgccgccctccccgccgacattacgttcacggccttcgaagccagccagggtaagaccccgcggggcgggcgcgacggcggcggcaagggcgcggcgggcgggttcgaacagcgcctggcctccgtcatggccggagacgccgccctggccctcgagtctatcgtgtcgatggccgtctttgacgagccgcccaccgacatctccgcgtggccgctgttcgagggccaggacacggccgcggcccgcgccaacgccgtcggggcgtacctggcgcgcgccgcgggactcgtgggggccatggtatttagcaccaactcggccctccatctcaccgaggtggacgacgccggcccggcggacccaaaggaccacagcaaaccctccttttaccgcttcttcctcgtgcccgggacccacgtggcggccaacccacaggtggaccgcgagggacacgtggtgcccgggttcgagggtcggcccaccgcgcccctcgtcggcggaacccaggaatttgccggcgagcacctggccatgctgtgtgggttttccccggcgctgctggccaagatgctgttttacctggagcgctgcgacggcgccgtgatcgtcgggcgccaggagatggacgtgtttcgatacgtcgcggactccaaccagaccgacgtgccctgtaacctatgcaccttcgacacgcgccacgcctgcgtacacacgacgctcatgcgcctccgggcgcgccatccaaagttcgccagcgccgcccgcggagccatcggcgtcttcgggaccatgaacagcatgtacagcgactgcgacgtgctgggaaactacgccgccttctcggccctgaagcgcgcggacggatccgagaccgcccggaccatcatgcaggagacgtaccgcgcggcgaccgagcgcgtcatggccgaactcgagaccctgcagtacgtggaccaggcggtccccacggccatggggcggctggagaccatcatcaccaaccgcgaggccctgcatacggtggtgaacaacgtcaggcaggtcgtggaccgcgaggtggagcagctgatgcgcaacctggtggaggggaggaacttcaagtttcgcgacggtctgggcgaggccaaccacgccatgtccctgacgctggacccgtacgcgtgcgggccgtgccccctgcttcagcttctcgggcggcgatccaacctcgccgtgtaccaggacctggccctgagtcagtgccacggggtgttcgccgggcagtcggtcgaggggcgcaactttcgcaatcaattccaaccggtgctgcggcggcgcgtgatggacatgtttaacaacgggtttctgtcggccaaaacgctgacggtcgcgctctcggagggggcggctatctgcgcccccagcctaacggccggccagacggcccccgccgagagcagcttcgagggcgacgttgcccgcgtgaccctggggtttcccaaggagctgcgcgtcaagagccgcgtgttgttcgcgggcgcgagcgccaacgcgtccgaggccgccaaggcgcgggtcgccagcctccagagcgcctaccagaagcccgacaagcgcgtggacatcctcctcggaccgctgggctttctgctgaagcagttccacgcggccatcttccccaacggcaagcccccggggtccaaccagccgaacccgcagtggttctggacggccctccaacgcaaccagcttcccgcccggctcctgtcgcgcgaggacatcgagaccatcgcgttcattaaaaagttttccctggactacggcgcgataaactttattaacctggcccccaacaacgtgagcgagctggcgatgtactacatggcaaaccagattctgcggtactgcgatcactcgacatacttcatcaacacccttacggccatcatcgcggggtcccgccgtccccccagcgtgcaggctgcggccgcgtggtccgcgcagggcggggcgggcctggaggccggggcccgcgcgctgatggacgccgtggacgcgcatccgggcgcgtggacgtccatgttcgccagctgcaacctgctgcggcccgtcatggcggcgcgccccatggtcgtgttggggttgagcatcagcaagtactacggcatggccggcaacgaccgtgtgtttcaggccgggaactgggccagcctgatgggcggcaaaaacgcgtgcccgctccttatttttgaccgcacccgcaagttcgtcctggcctgtccccgggccgggtttgtgtgcgcggcctcaagcctcggcggcggagcgcacgaaagctcgctgtgcgagcagctccggggcattatctccgagggcggggcggccgtcgccagtagcgtgttcgtggcgaccgtgaaaagcctggggccccgcacccagcagctgcagatcgaggactggctggcgctcctggaggacgagtacctaagcgaggagatgatggagctgaccgcgcgtgccctggagcgcggcaacggcgagtggtcgacggacgcggccctggaggtggcgcacgaggccgaggccctagtcagccaactcggcaacgccggggaggtgtttaactttggggattttggctgcgaggacgacaacgcgacgccgttcggcggcccgggggccccgggaccggcatttgccggccgcaaacgggcgttccacggggatgacccgtttggggaggggccccccgacaaaaagggagacctgacgttggatatgctgtga

SEQ ID NO: 68 - protein, 1196 aa, Herpes Virus
METKPKTATTIKVPPGPLGYVYARACPSEGIELLALLSARSGDADVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGENFTYPLPFFNRPLNRLLFEAVVGPAAVALRCRNVDAVARAAAHLAFDENHEGAALPADITFTAFEASQGKTPRGGRDGGGKGPAGGFEQRLASVMAGDAALALESIVSMAVFDEPPTDISAWPLCEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASNLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 69 - DNA, 1323 bp, Herpes Virus
aaaaccaagaagaaatccacccccaaaggcaaaacccccgtcggggccgcggtccccgcctccgttccggagcctgtcctcgcctcggcaccccccgacccggccgggccgccggtcgccgaggcgggcgaggacgacgggcccacggttccggcgtcctcacaggccctcgaggcgctgaagactcgccgctcgcccgagcccccgggcgcagacctcgcccagctgttcgaggcccacccaaacgtggccgccacggcggttaagttcaccgcgtgctccgccgccctggcccgcgaggtcgccgcgtgttcgcggctcaccatcagcgccttacggtcgccgtatccggcctctccggggctgctggagctctgtgttatttttttctttgaacgcgtcctcgcctttctcatcgagaacggggcccggacgcacacccaggccggggtggccggcccggccgccgccctgctggagtttaccctgaacatgctgccctggaaaacggccgtgggggactttctggcctccacgcgcctgagcctggccgacgtggccgcccatctgcccctcgtccagcacgtgctggacgaaaactctctgatcggtcgcctggcgctggcgaagctgatccttgtggctagggatgtcattcgggagacggacgccttttacggggaactcgcggacctggagctgcagcttcgcgcggccccgccggccaatctgtatacacgcctcggcgagtggcttctggagcgctcgcaggcccacccggacaccctttttgcccccgccaccccgacgcacccagaaccgcttctgtatagagtccaggctctggccaaatttgcccgtggcgaagagattagggtggaggcggaggatcgccagatgcgcgaggccctcgacgccctcgctcgcggggtcgacgcggtctcacagcacgccgggcccctcggcgtaatgcccgccccggccggggcggccccgcagggggctccgcgcccaccccccctgggccccgaggccgttcaggttcggctggaggaggtgcggacccaggcccgtcgggcgatcgagggcgcggttaaggagtacttttaccggggggccgtatacagcgccaaggctctacaggccagcgacaacaacgaccgccggtttcacgtggcttcggccgccgtcgtgcccgtggtccagctgctcgagtccctgcctgtcttcgaccagcacacgcgggacatcgcgcagcgcgccgccattcccgccccgcccccgatcgcgaccagccccacggccatcctgttgcgggatctg

SEQ ID NO: 70 - protein, 441 aa, Herpes Virus
KTKKKSTPKGKTPVGAAVPASVPEPVLASAPPDPAGPPVAEAGEDDGPTVPASSQALEALKTRRSPEPPGADLAQLFEAHPNVAATAVKFTACSAALAREVAACSRLTISALRSPYPASPGLLELCVIFFFERVLAFLIENGARTHTQAGVAGPAAALLEFTLNMLPWKTAVGDFLASTRLSLADVAAHLPLVQHVLDENSLIGRLALAKLILVARDVIRETDAFYGELADLELQLRAAPPANLYTRLGEWLLERSQAHPDTLFAPATPTHPEPLLYRVQALAKFARGEEIRVEAEDRQMREALDALARGVDAVSQHAGPLGVMPAPAGAAPQGAPRPPPLGPEAVQVRLEEVRTQARRAIEGAVKEYFYRGAVYSAKALQASDNNDRRFHVASAAVVPVVQLLESLPVFDQHTRDIAQRAAIPAPPPIATSPTAILLRDL

SEQ ID NO: 71 - DNA, 9495 bp, Herpes Virus
atgggtggcggaaacaacactaaccccgggggtccggtccataaacaggccgggtctctggccagcagggcacatatgatcgcgggcaccccaccgcactccacgatggaacgcgggggggatcgcgacatcgtggtcaccggtgctcggaaccagttcgcgcccgacctggagccgggggggtcggtatcgtgcatgcgctcgtcgctgtcctttctcagcctcatatttgatgtgggccctcgcgacgtcctgtccgcggaggccatcgagggatgtttggtcgaggggggcgagtggacgcgcgcgaccgcgggccctgggccgccgcgcatgtgttcgatcgtcgagctccccaacttcctcgagtacccaggggcgcgcggcggactgcgctgtgtcttctcgcgcgtatacggcgaggtgggcttcttcggggagcccgcggcgggcctgctggagacacaatgccccgcacacacgttcttcgccggcccgtgggccctgcgccccctgtcgtacacgctcctaaccattggccccctagggatggggctgttcagggacggcgacaccgcatacctttttgacccgcacggccttccggagggcacccccgcgttcatcgccaaagtgcgggcgggggacatgtatccatacctgacgtattacacccgcgatcgcccggacgtacggtgggcgggagccatggtgtttttcgtgccgtcgggcccggaacccgcggctcctgcggacttgacggccgcggctctgcatctttacggggccagcgagacttacctgcaggacgaagcgttcagcgaacggcgcgtggccatcacgcaccccctgcggggcgagatcgcgggcctgggggagccctgcgtcggcgtgggcccccgggagggggtagggggcccggggccacacccgcccacagccgcccagtcgccgccaccgacccgggcccgtcgcgacgacagggcctccgagacatcccgggggacggccggtccgtcggcaaaaccagaggccaagcgcccgaatcgggcgcccgacgatgtatgggcggtggccctgaagggtaccccacccacggatcccccctccgccgacccaccctccgccgacccaccctccgcgatcccaccaccgcctccctccgcccccaagacccccgccgcagaggcggccgaagaagatgacgacgacatgcgggtcctggagatgggcgtcgtcccggttggtcggcaccgggcacgctactcggccggccttcccaagcgccgccgacccacctggactccgccttccagcgtcgaagacctgacttcgggggagaaaacgaaacgctcggccccccctgccaaaaccaagaagaaatccacccccaaaggcaaaacccccgtcggggccgcggtccccgcctccgttccggagcctgtcctcgcctcggcaccccccgacccggccgggccgccggtcgccgaggcgggcgaggacgacgggcccacggttccggcgtcctcacaggccctcgaggcgctgaagactcgccgctcgcccgagcccccgggcgcagacctcgcccagctgttcgaggcccacccaaacgtggccgccacggcggttaagttcaccgcgtgctccgccgccctggcccgcgaggtcgccgcgtgttcgcggctcaccatcagcgccttacggtcgccgtatccggcctctccggggctgctggagctctgtgttatttttttctttgaacgcgtcctcgcctttctcatcgagaacggggcccggacgcacacccaggccggggtggccggcccggccgccgccctgctggagtttaccctgaacatgctgccctggaaaacggccgtgggggactttctggcctccacgcgcctgagcctggccgacgtggccgcccatctgcccctcgtccagcacgtgctggacgaaaactctctgatcggtcgcctggcgctggcgaagctgatccttgtggctagggatgtcattcgggagacggacgccttttacggggaactcgcggacctggagctgcagcttcgcgcggccccgccggccaatctgtatacacgcctcggcgagtggcttctggagcgctcgcaggcccacccggacaccctttttgcccccgccaccccgacgcacccagaaccgcttctgtatagagtccaggctctggccaaatttgcccgtggcgaagagattagggtggaggcggaggatcgccagatgcgcgaggccctcgacgccctcgctcgcggggtcgacgcggtctcacagcacgccgggcccctcggcgtaatgcccgccccggccggggcggccccgcagggggctccgcgcccaccccccctgggccccgaggccgttcaggttcggctggaggaggtgcggacccaggcccgtcgggcgatcgagggcgcggttaaggagtacttttaccggggggccgtatacagcgccaaggctctacaggccagcgacaacaacgaccgccggtttcacgtggcttcggccgccgtcgtgcccgtggtccagctgctcgagtccctgcctgtcttcgaccagcacacgcgggacatcgcgcagcgcgccgccattcccgccccgcccccgatcgcgaccagccccacggccatcctgttgcgggatctgatccagcggggccagacgctggacgcccccgaggacctggcggcctggctctccgtcctgacggacgccgccaaccaagggctgatagaacgcaagccactggacgagctggcgcgcagcatccgcgacattaacgaccaacaggcgcgccgcagctcgggtctggccgagctgcggcgcttcgacgccctagatgcggccctgggccagcagctggacagcgacgcggcctttgttcctgcgcccggcgcgtcgccctaccccgacgacggcgggctgtcgccagaggccacgcgcatggccgaggaagcgctgcggcaggcgcgggccatggatgccgccaagctgacggcagagctcgcccccgatgcgcgtgcccgtttgcgggagcgcgcgcgctccctggaggcaatgctcgagggagcgcgggagcgggcgaaggtggcccgcgacgcccgggagaagttcttgcacaaactccagggggtcctgcgccccctccctgactttgtggggctaaaggcctgtccggccgtcctggcgaccctgcgggcctccctgccggcgggctggtcggacctccccgaggccgttcggggggcgccccctgaggttacggcggcgctgcgggcggacatgtgggggctgctggggcagtaccgagatgccctggagcacccgactccggacacggcgacggctctgtctggcttgcatcccagcttcgtggtggtgctgaagaacctgttcgccgacgccccagagactccgtttctcttgcagttcttcgccgatcacgccccgatcatagcccacgccgtctcgaacgccatcaacgccggcagcgccgccgtcgcaacggcagaccctgcgtcgacggtggatgcggccgtgcgggcgcaccgcgtcctggtcgacgcggtgacggccctgggcgcggccgccagcgacccggcctcccccctggccttcctagcggccatggccgacagcgccgcgggatacgtcaaggcgactcggttggccctggacgcgcgggtggccatcgcccagctcacgaccttagggtcggctgccgccgaccttgtcgtccaggtgcgccgggccgccaaccaaccggagggagagcatgcctccctgatccaggccgcgacgcgcgcgaccaccggcgcgcgggaaagcctcgcgggccacgagggcaggttcgggggcctgttgcacgccgaagggacggccggggaccactcccccagcgggcgcgccctgcaggagctgggaaaggtcatcggcgccacgcgacgccgcgccgacgaacttgaggccgccaccgccgacctcagagagaagatggcggcccagcgcgcccgcagtagccacgagcgctgggccgccgacgtggaggccgtgctggaccgcgtggaaagcggtgccgagtttgacgtggtcgagctccgtcgcctgcaggcgctggcgggcacgcacggctacaacccccgggacttccgaaagcgggccgaacaggcgctgggaaccaacgccaaggcggtgacccttgccctggagacggcccttgcgtttaacccatacacccccgagaaccagcgccaccccatgctccccccgctcgcagccattcaccgcatcgactggagcgcggccttcggggccgcggccgacacgtacgccgacatgtttcgggtggacaccgagcccctggcgcggcttctgcggctggcgggggggctgctggagcgggcccaggcgaacgacgggtttatcgactaccacgaggccgtcctacacctgtcggaagacttggggggcgtgccggccctgcgccagtacgtgccgttttttcaaaagggctacgccgagtacgtggatatccgcgatcgcctggacgccctccgggccgacgcgcggcgcgcgatcggaagcgtggcgctggacctggccgccgccgcggaggagatatccgcggtgcgcaacgacccggcggcggccgccgagcttgtccgggcaggggtcaccctgccctgcccgagcgaggacgcgctggtggcgtgcgtggcggcgctggagcgcgtggaccagagccccgtgaaggacacggcgtacgcgcactacgtcgcattcgtgacccgacaggacctggccgataccaaggacgccgtggtgcgcgccaaacagcagcgcgccgaagccaccgagcgggtcacggcggggctgcgggaggtgctggccgcgcgcgagcgccgggcccagctcgaggccgagggtctggccaatctgaagaccctgctgaaggtggtcgccgtcccggcgaccgtggccaagacgctggaccaggcgcgctcggcggaggagatcgcggatcaggtcgaaattctggtggaccagacggagaaggcgcgcgagctcgacgtgcaggcggtcgcctggttggaacatgcccagcgtacctttgagacgcacccgctaagcgcggccagcggcgacggcccgggcctcctgacgcgacagggcgcgcgcctgcaggcgctcttcgacacccgtcgccgcgtcgaggccctgcggaggtctctcgaggaggccgaggcggagtgggacgaggtatggggtcgcttcggccgcgttcgcgggggggcctggaaatcgcccgagggatttcgcgcggcatgcgagcagcttcgcgccctgcaggacaccaccaacactgtgtcggggctgcgagcccagcgggactacgagcgccttcccgccaagtaccagggcgtcctgggcgccaagagcgccgagcgggccggggccgtggaggagctcggggggcgcgtggcccaacacgccgacctgagcgcccggctgcgggacgaggtggtgccaagggtggcctgggagatgaactttgacaccctggggggcctgttggcggaattcgacgcggtggccggggacctggccccatgggcggtggaggagttccggggcgcgcgggagctcatccaacgccgcatgggcttatatagcgcgtacgccaaggccacaggccagacgggcgcgggcgcggcggccgcgcccgcgcccctgctcgtggatcttcgcgccctagacgcccgcgcccgggcgtccgccccacccggccaagaggccgacccgcagatgctgcgccgccggggcgaggcgtacctgcgagtgagcggaggcccggggcccctggtgctgcgcgaggccaccagtacgctggatcggccgttcgcccccagctttttggtcccggatggaacgccactgcagtacgcgctctgcttcccggccgtgaccgacaagctcggcgcgctgctgatgtgtcccgaggcggcatgcattcgccccccgcttccgacggacaccctggagtcggcctcgaccgtcacggccatgtacgtcctcaccgtcatcaaccggcttcagctggccctcagcgacgcccaggccgccaactttcagctcttcggacgctttgtgcgccaccgccaggcgagatggggggcctcgatggatgcggcggccgagctctacgtcgccctcgtcgcgaccactctcacgcgcgagtttgggtgtcgctgggcccagctggaatgggggggtgacgcggcggccccggggccgccgctcggaccccagagctccactaggcaccgcgtttcctttaacgagaacgacgtgctggtggcgctggtggccagctccccggaacacatttacaccttttggcgcctggatctggttcgccaacacgagtacatgcatctcaccctcccccgtgcgtttcagaacgcagcagattccatgctattcgtgcagcgcctgaccccgcatccagacgcccgcatccgcgtgctgccagcgttttcggccggaggccctccgacccggggcctcatgttcggcacgcggctggcagactggcgccgcggcaagttgtccgaaaccgaccccctggcgccctggcgctcggtccccgagctgggaaccgagcgcggcgccgcgctgggaaagctgagtcccgcccaggcgctggcggcggtgagcgtcctcgggcgcatgtgtctcccaagcaccgctctggtcgctctttggacctgcatgtttccggacgactacacagagtatgacagtttcgacgcccttctgaccgcgcgtctggaatctgggcagacgctgagcccctcgggggggcgcgaggcgtcaccccccgctccccccaacgccctctaccggcccacgggccagcacgtcgccgtgccggccgccgccacccaccgcacccccgcggcgcgcgttacggccatggacctggtgctggcggcagtgctcctgggcgcgcccgtcgtcgtggcgctccgcaacaccacggccttttcccgcgagtcggagctggagctgtgtctcacgctgtttgactcacgcgctcgcgggccggacgccgccttgcgcgatgccgtgtcgtccgacatcgagacgtgggccgtccgcctcctgcacgccgacctgaacccgatcgaaaacgcgtgtctggcggcacagctcccgcgcctgtccgcgctcatcgccgagcggcccctcgcccggggcccgccgtgtctggtgctcgtggacatctccatgaccccggtcgcggtgttgtgggaaaacccggacccccccggcccccccgacgtgcggtttgttggcagcgaggccaccgaggagctcccgtttgtggcgggcggcgaggacgtcctcgccgccagcgccaccgacgaggaccccttcctcgcgcgagctatcctcgggcggccgttcgacgcctccctcctgtcgggggagctattcccggggcatccggtgtaccagcgcgcccccgacgaccagagcccctcggtcccaaacccgacccccggccctgtggaccttgttggggcggagggctcgttggggcccggaagcctggcccccacgctattcaccgacgccacccccggcgagcccgtcccccctcgcatgtgggcatggattcacggcctggaggagctcgcgtctgacgactccggcggccccgcgcccctccttgccccggaccccctttcgcccaccgccgatcagtccgtccccacgtcccagtgtgcaccgcggccccctgggccggcagtcacggctcgcgaagcacgaccgggcgtcccggccgaaagcacgcggccggcgcccgtgggcccccgcgacgacttccggcgcttgccgtccccccaaagttccccggcgccccccgatgccaccgccccccgcccccccgcctcctcccgcgcttctgccgcttcttcgtccgggtcgcgcgcgcgccgacaccgccgggcacgctccctggcgcgcgccacccaggcttccgcgaccacccagggttggcggccgcctgccctccccgacacggtcgccccggttaccgatttcgcgcgccccccggcccctcccaaacccccagagccagcgccccacgctttggtgtctggtgtgcccctcccgctcgggccccaggccgccggccaggcttctcccgctctccctatcgatcccgttccgcccccggtcgcaaccggcacggttttgcccgggggcgaaaaccgccgccccccgctaacctcgggtcccgcgccaaccccccccagggttcccgtaggcgggccgcagcggcgccttacgcgccccgctgtcgcgtcgctgtccgaatcgcgggaatccctcccttcaccctgggaccccgccgaccccacggcccctgttttaggccgcaacccggccgagccgacctcatcctctcccgcaggtccctctcccccgcctcccgcggtccaacccgtcgccccgcccccgacgtcaggcccgccccccacatacttgacgctggagggcggtgttgcgcccggaggcccggtttcccgccgccccactacacggcagccggtggccacgcccaccacatctgcgcgcccccgggggcatttgaccgtcagccgcctgtccgcgccccaaccccagccccagccccagccccagccccagccccagccccagccccagccccagccccagccccagccccagccccagccccagccccagccccagccccaaccccaaccccagccccaaccccaaccccaaccccagccccaaccccagccccaaccccagccccaaccccagccccaaccccagccccaaccccagccccaaaacgggcatgtagcacccggggagtatccggcggttcggttccgggcaccgcaaaaccgcccatccgtcccggcttccgcgtcttccacaaatccacgcacgggcagctccttgtctggggtgtcttcgtgggcatcctccctcgcgctacacatcgacgctacccccccgcccgtgtcgctgcttcagaccctgtatgtctctgacgacgaagactccgacgccacctcgttgttcctctcggattccgaggccgaggcgctcgacccactccctggggaaccacactcccccataaccaacgaaccattcagtgcgttatccgccgatgactcccaagaggtgacgcgcttacaattcggccccccgcccgtatcggcaaacgcagttctgtcgcgacgctacgtgcaacgcaccggtcgtagcgccctggcggtactgatccgcgcctgttaccgcctacaacagcagttacagcggacccgccgggcgctgcttcatcacagcgacgccgtgctgaccagcctacatcacgtgcgcatgttactgggctag

SEQ ID NO: 72 - protein, 3164 aa, Herpes Virus
MGGGNNTNPGGPVHKQAGSLASRAHMIAGTPPHSTMERGGDRDIVVTGARNQFAPDLEPGGSVSCMRSSLSFLSLIFDVGPRDVLSAEAIEGCLVEGGEWTRATAGPGPPRMCSIVELPNFLEYPGARGGLRCVFSRVYGEVGFFGEPAAGLLETQCPAHTFFAGPWALRPLSYTLLTIGPLGMGLFRDGDTAYLFDPHGLPEGTPAFIAKVRAGDMYPYLTYYTRDRPDVRWAGAMVFFVPSGPEPAAPADLTAAALHLYGASETYLQDEAFSERRVAITHPLRGEIAGLGEPCVGVGPREGVGGPGPHPPTAAQSPPPTRARRDDRASETSRGTAGPSAKPEAKRPNRAPDDVWAVALKGTPPTDPPSADPPSADPPSAIPPPPPSAPKTPAAEAAEEDDDDMRVLEMGVVPVGRHRARYSAGLPKRRRPTWTPPSSVEDLTSGEKTKRSAPPAKTKKKSTPKGKTPVGAAVPASVPEPVLASAPPDPAGPPVAEAGEDDGPTVPASSQALEALKTRRSPEPPGADLAQLFEAHPNVAATAVKFTACSAALAREVAACSRLTISALRSPYPASPGLLELCVIFFFERVLAFLIENGARTHTQAGVAGPAAALLEFTLNMLPWKTAVGDFLASTRLSLADVAAHLPLVQHVLDENSLIGRLALAKLILVARDVIRETDAFYGELADLELQLRAAPPANLYTRLGEWLLERSQAHPDTLFAPATPTHPEPLLYRVQALAKFARGEEIRVEAEDRQMREALDALARGVDAVSQHAGPLGVMPAPAGAAPQGAPRPPPLGPEAVQVRLEEVRTQARRAIEGAVKEYFYRGAVYSAKALQASDNNDRRFHVASAAVVPVVQLLESLPVFDQHTRDIAQRAAIPAPPPIATSPTAILLRDLIQRGQTLDAPEDLAAWLSVLTDAANQGLIERKPLDELARSIRDINDQQARRSSGLAELRRFDALDAALGQQLDSDAAFVPAPGASPYPDDGGLSPEATRMAEEALRQARAMDAAKLTAELAPDARARLRERARSLEAMLEGARERAKVARDAREKFLHKLQGVLRPLPDFVGLKACPAVLATLRASLPAGWSDLPEAVRGAPPEVTAALRADMWGLLGQYRDALEHPTPDTATALSGLHPSFVVVLKNLFADAPETPFLLQFFADHAPIIAHAVSNAINAGSAAVATADPASTVDAAVRAHRVLVDAVTALGAAASDPASPLAFLAAMADSAAGYVKATRLALDARVAIAQLTTLGSAAADLVVQVRRAANQPEGEHASLIQAATRATTGARESLAGHEGRFGGLLHAEGTAGDHSPSGRALQELGKVIGATRRRADELEAATADLREKMAAQRARSSHERWAADVEAVLDRVESGAEFDVVELRRLQALAGTHGYNPRDFRKRAEQALGTNAKAVTLALETALAFNPYTPENQRHPMLPPLAAIHRIDWSAAFGAAADTYADMFRVDTEPLARLLRLAGGLLERAQANDGFIDYHEAVLHLSEDLGGVPALRQYVPFFQKGYAEYVDIRDRLDALRADARRAIGSVALDLAAAAEEISAVRNDPAAAAELVRAGVTLPCPSEDALVACVAALERVDQSPVKDTAYAHYVAFVTRQDLADTKDAVVRAKQQRAEATERVTAGLREVLAARERRAQLEAEGLANLKTLLKVVAVPATVAKTLDQARSAEEIADQVEILVDQTEKARELDVQAVAWLEHAQRTFETHPLSAASGDGPGLLTRQGARLQALFDTRRRVEALRRSLEEAEAEWDEVWGRFGRVRGGAWKSPEGFRAACEQLRALQDTTNTVSGLRAQRDYERLPAKYQGVLGAKSAERAGAVEELGGRVAQHADLSARLRDEVVPRVAWEMNFDTLGGLLAEFDAVAGDLAPWAVEEFRGARELIQRRMGLYSAYAKATGQTGAGAAAAPAPLLVDLRALDARARASAPPGQEADPQMLRRRGEAYLRVSGGPGPLVLREATSTLDRPFAPSFLVPDGTPLQYALCFPAVTDKLGALLMCPEAACIRPPLPTDTLESASTVTAMYVLTVINRLQLALSDAQAANFQLFGRFVRHRQARWGASMDAAAELYVALVATTLTREFGCRWAQLEWGGDAAAPGPPLGPQSSTRHRVSFNENDVLVALVASSPEHIYTFWRLDLVRQHEYMHLTLPRAFQNAADSMLFVQRLTPHPDARIRVLPAFSAGGPPTRGLMFGTRLADWRRGKLSETDPLAPWRSVPELGTERGAALGKLSPAQALAAVSVLGRMCLPSTALVALWTCMFPDDYTEYDSFDALLTARLESGQTLSPSGGREASPPAPPNALYRPTGQHVAVPAAATHRTPAARVTAMDLVLAAVLLGAPVVVALRNTTAFSRESELELCLTLFDSRARGPDAALRDAVSSDIETWAVRLLHADLNPIENACLAAQLPRLSALIAERPLARGPPCLVLVDISMTPVAVLWENPDPPGPPDVRFVGSEATEELPFVAGGEDVLAASATDEDPFLARAILGRPFDASLLSGELFPGHPVYQRAPDDQSPSVPNPTPGPVDLVGAEGSLGPGSLAPTLFTDATPGEPVPPRMWAWIHGLEELASDDSGGPAPLLAPDPLSPTADQSVPTSQCAPRPPGPAVTAREARPGVPAESTRPAPVGPRDDFRRLPSPQSSPAPPDATAPRPPASSRASAASSSGSRARRHRRARSLARATQASATTQGWRPPALPDTVAPVTDFARPPAPPKPPEPAPHALVSGVPLPLGPQAAGQASPALPIDPVPPPVATGTVLPGGENRRPPLTSGPAPTPPRVPVGGPQRRLTRPAVASLSESRESLPSPWDPADPTAPVLGRNPAEPTSSSPAGPSPPPPAVQPVAPPPTSGPPPTYLTLEGGVAPGGPVSRRPTTRQPVATPTTSARPRGHLTVSRLSAPQPQPQPQPQPQPQPQPQPQPQPQPQPQPQPQPQPQPQPQPQPQPQPQPQPQPQPQPQPQPQPQPQPQPQNGHVAPGEYPAVRFRAPQNRPSVPASASSTNPRTGSSLSGVSSWASSLALHIDATPPPVSLLQTLYVSDDEDSDATSLFLSDSEAEALDPLPGEPHSPITNEPFSALSADDSQEVTRLQFGPPPVSANAVLSRRYVQRTGRSALAVLIRACYRLQQQLQRTRRALLHHSDAVLTSLHHVRMLLG

SEQ ID NO: 73 - DNA, 1128 bp, Herpes Virus
ttccagcctgccgtctccagcctgctgcagctcggggagcagccctccgccggcgcccagcagcggctgctggctctgctgcagcagacgtggacgttgatccagaataccaattcgccctccgtggtgatcaacaccctgatcgacgctgggttcacgccctcgcactgcacgcactacctttcggccctggaggggtttctggcggcgggcgtccccgcgcggacgcccaccggccacggactcggcgaagtccagcagctctttgggtgcattgccctcgcggggtcgaacgtgtttgggttggcgcgggaatacgggtactatgccaactacgtaaaaactttcaggcgggtccagggcgccagcgagcacacgcacgggcggctctgcgaggcggtcggcctgtcggggggcgttctaagccagacgctggcgcgtatcatgggtccggccgtgccgacggaacatctggcgagcctgcggcgggcgctcgtgggggagtttgagacggccgagcgccgctttagttccggtcaacccagccttctccgcgagacggcgctcatctggatcgacgtgtatggtcagacccactgggacatcacccccaccaccccggccacgccgctgtccgcgcttctccccgtcgggcagcccagccacgccccctctgtccacctggccgcggcgacccagatccgcttccccgccctcgagggcattcaccccaacgtcctcgccgacccgggcttcgtcccctacgttctggccctggtggtcggggacgcgctgagggccacgtgtagcgcggcctaccttccccgcccggtcgagttcgccctgcgtgtgttggcctgggcccgggactttgggctgggctatctccccacggttgagggccatcgcaccaaactgggcgcgctgatcaccctcctcgaaccggccgcccggggcggcctcggccccactatgcagatggccgacaacatagagcagctgctccgggagctgtacgtgatctccaggggtgccgtcgagcagctgcggcccctggtccagctgcagccccccccgccccccgaggtgggcaccagcctcctgttgattagcatgtacgccctggcc

SEQ ID NO: 74 - protein, 376 aa, Herpes Virus
FQPAVSSLLQLGEQPSAGAQQRLLALLQQTWTLIQNTNSPSVVINTLIDAGFTPSHCTHYLSALEGFLAAGVPARTPTGHGLGEVQQLFGCIALAGSNVFGLAREYGYYANYVKTFRRVQGASEHTHGRLCEAVGLSGGVLSQTLARIMGPAVPTEHLASLRRALVGEFETAERRFSSGQPSLLRETALIWIDVYGQTHWDITPTTPATPLSALLPVGQPSHAPSVHLAAATQIRFPALEGIHPNVLADPGFVPYVLALVVGDALRATCSAAYLPRPVEFALRVLAWARDFGLGYLPTVEGHRTKLGALITLLEPAARGGLGPTMQMADNIEQLLRELYVISRGAVEQLRPLVQLQPPPPPEVGTSLLLISMYALA

SEQ ID NO: 75 - DNA, 1083 bp, Herpes Virus
ctgattcgccaactggaggacgccatcgtgctgctgcggctgcacatgcgcacgctctccgcctttttcgagtgtcggttcgagagcgacgggcgccgcctgtatgcggtggtcggggacacgcccgaccgcctggggccctggccccccgaggccatgggggacgcggtgagtcagtactgcagcatgtatcacgacgccaagcgcgcgctggtcgcgtccctcgcgagcctgcgttccgtcatcaccgaaaccacggcgcacctgggggtgtgcgacgagctggcggcccaggtgtcgcacgaggacaacgtgctggccgtggtccggcgcgaaattcacgggtttctgtccgtcgtgtccggcattcacgcccgggcgtcgaagctgctgtcgggagaccaggtccccgggttttgcttcatgggtcagtttctagcgcgctggcggcgtctgtcggcctgctatcaagccgcgcgcgcggccgcgggacccgagcccgtggccgagtttgtccaggaactccacgacacgtggaagggcctgcagacggagcgcgccgtggtcgtggcgcccttggtcagctcggccgaccagcgcgccgcggccatccgagaggtaatggcgcatgcgcccgaggacgcccccccgcaaagccccgcggccgaccgcgtcgtgcttacgagccgtcgcgacctaggggcctggggggactacagcctcggccccctgggccagacgaccgcggttccggactccgtggatctgtctcgccaggggctggccgttacgctgagtatggattggttactgatgaacgagctcctgcgggtcaccgacggcgtgtttcgcgcttccgcgtttcgtccgttagccggaccggagtctcccagggacctggaggtccgcgacgccggaaacagtctccccgcgcctatgcccatggacgcacagaagccggaggcctatgggcacggcccacgccaggcggaccgcgagggggcgcctcattccaacacccccgtcgaggacgacgagatgatcccggaggacaccgtcgcgccacccacggacttgccgtta

SEQ ID NO: 76 - protein, 361 aa, Herpes Virus
LIRQLEDAIVLLRLHMRTLSAFFECRFESDGRRLYAVVGDTPDRLGPWPPEAMGDAVSQYCSMYHDAKRALVASLASLRSVITETTAHLGVCDELAAQVSHEDNVLAVVRREIHGFLSVVSGIHARASKLLSGDQVPGFCFMGQFLARWRRLSACYQAARAAAGPEPVAEFVQELHDTWKGLQTERAVVVAPLVSSADQRAAAIREVMAHAPEDAPPQSPAADRVVLTSRRDLGAWGDYSLGPLGQTTAVPDSVDLSRQGLAVTLSMDWLLMNELLRVTDGVFRASAFRPLAGPESPRDLEVRDAGNSLPAPMPMDAQKPEAYGHGPRQADREGAPHSNTPVEDDEMIPEDTVAPPTDLPL

SEQ ID NO: 77 - DNA, 3372 bp, Herpes Virus
atggcagaccgcggtctcccgtccgaggcccccgtcgtcacgacctcacccgccggtccgccctcggacggacctatgcagcgcctattggcgagcctagccggccttcgccaaccgccaacccccacggccgagacggcaaacggggcggacgacccggcgtttctggccacggccaagctgcgcgccgccatggcggcgtttctgttgtcgggaacggccatcgccccggcagacgcgcgggactgctggcggccgctgctggaacacctgtgcgcgctccaccgggcccacgggcttccggagacggcgctcttggccgagaacctccccgggttgctcgtacaccgcttggtggtggctctccccgaggcccccgaccaggccttccgggagatggaggtcatcaaggacaccatcctcgcggtcaccggctccgacacgtcccatgcgctggattccgccggcctgcgcaccgcggcggccctggggccggtccgcgtccgccagtgcgccgtggagtggatagaccgctggcaaaccgtcaccaagagctgcttggccatgagcccgcggacctccatcgaggcccttggggagacgtcgctcaagatggcgccggtcccgttggggcagcccagcgcgaaccttaccaccccggcgtacagcctgctcttccccgccccgttcgtgcaagagggcctccggttcttggccctggtgagtaatcgggtgacgctgttctcggcgcacctccagcgcatagacgacgcgaccctcactcccctcacacgggccctctttacgttggccctggtggacgagtacctgacgacccccgagcggggggctgtggtcccgccgcccctgttggcgcagtttcagcacaccgtgcgggagatcgacccggccataatgattccgccgctcgaggccaacaagatggttcgcagccgcgaggaggtgcgcgtgtcgacggccctcagccgcgtcagcccgcgctcggcctgtgcgcccccggggacgctaatggcgcgcgtgcggacggacgtggccgtgtttgatcccgacgtgccgttcctgagttcgtcggcactggcagtcttccagcctgccgtctccagcctgctgcagctcggggagcagccctccgccggcgcccagcagcggctgctggctctgctgcagcagacgtggacgttgatccagaataccaattcgccctccgtggtgatcaacaccctgatcgacgctgggttcacgccctcgcactgcacgcactacctttcggccctggaggggtttctggcggcgggcgtccccgcgcggacgcccaccggccacggactcggcgaagtccagcagctctttgggtgcattgccctcgcggggtcgaacgtgtttgggttggcgcgggaatacgggtactatgccaactacgtaaaaactttcaggcgggtccagggcgccagcgagcacacgcacgggcggctctgcgaggcggtcggcctgtcggggggcgttctaagccagacgctggcgcgtatcatgggtccggccgtgccgacggaacatctggcgagcctgcggcgggcgctcgtgggggagtttgagacggccgagcgccgctttagttccggtcaacccagccttctccgcgagacggcgctcatctggatcgacgtgtatggtcagacccactgggacatcacccccaccaccccggccacgccgctgtccgcgcttctccccgtcgggcagcccagccacgccccctctgtccacctggccgcggcgacccagatccgcttccccgccctcgagggcattcaccccaacgtcctcgccgacccgggcttcgtcccctacgttctggccctggtggtcggggacgcgctgagggccacgtgtagcgcggcctaccttccccgcccggtcgagttcgccctgcgtgtgttggcctgggcccgggactttgggctgggctatctccccacggttgagggccatcgcaccaaactgggcgcgctgatcaccctcctcgaaccggccgcccggggcggcctcggccccactatgcagatggccgacaacatagagcagctgctccgggagctgtacgtgatctccaggggtgccgtcgagcagctgcggcccctggtccagctgcagccccccccgccccccgaggtgggcaccagcctcctgttgattagcatgtacgccctggccgcccggggggtgctgcaggacctcgccgagcgcgcagaccccctgattcgccaactggaggacgccatcgtgctgctgcggctgcacatgcgcacgctctccgcctttttcgagtgtcggttcgagagcgacgggcgccgcctgtatgcggtggtcggggacacgcccgaccgcctggggccctggccccccgaggccatgggggacgcggtgagtcagtactgcagcatgtatcacgacgccaagcgcgcgctggtcgcgtccctcgcgagcctgcgttccgtcatcaccgaaaccacggcgcacctgggggtgtgcgacgagctggcggcccaggtgtcgcacgaggacaacgtgctggccgtggtccggcgcgaaattcacgggtttctgtccgtcgtgtccggcattcacgcccgggcgtcgaagctgctgtcgggagaccaggtccccgggttttgcttcatgggtcagtttctagcgcgctggcggcgtctgtcggcctgctatcaagccgcgcgcgcggccgcgggacccgagcccgtggccgagtttgtccaggaactccacgacacgtggaagggcctgcagacggagcgcgccgtggtcgtggcgcccttggtcagctcggccgaccagcgcgccgcggccatccgagaggtaatggcgcatgcgcccgaggacgcccccccgcaaagccccgcggccgaccgcgtcgtgcttacgagccgtcgcgacctaggggcctggggggactacagcctcggccccctgggccagacgaccgcggttccggactccgtggatctgtctcgccaggggctggccgttacgctgagtatggattggttactgatgaacgagctcctgcgggtcaccgacggcgtgtttcgcgcttccgcgtttcgtccgttagccggaccggagtctcccagggacctggaggtccgcgacgccggaaacagtctccccgcgcctatgcccatggacgcacagaagccggaggcctatgggcacggcccacgccaggcggaccgcgagggggcgcctcattccaacacccccgtcgaggacgacgagatgatcccggaggacaccgtcgcgccacccacggacttgccgttaactagttaccaataa

SEQ ID NO: 78 - protein, 1123 aa, Herpes Virus
MADRGLPSEAPVVTTSPAGPPSDGPMQRLLASLAGLRQPPTPTAETANGADDPAFLATAKLRAAMAAFLLSGTAIAPADARDCWRPLLEHLCALHRAHGLPETALLAENLPGLLVHRLVVALPEAPDQAFREMEVIKDTILAVTGSDTSHALDSAGLRTAAALGPVRVRQCAVEWIDRWQTVTKSCLAMSPRTSIEALGETSLKMAPVPLGQPSANLTTPAYSLLFPAPFVQEGLRFLALVSNRVTLFSAHLQRIDDATLTPLTRALFTLALVDEYLTTPERGAVVPPPLLAQFQHTVREIDPAIMIPPLEANKMVRSREEVRVSTALSRVSPRSACAPPGTLMARVRTDVAVFDPDVPFLSSSALAVFQPAVSSLLQLGEQPSAGAQQRLLALLQQTWTLIQNTNSPSVVINTLIDAGFTPSHCTHYLSALEGFLAAGVPARTPTGHGLGEVQQLFGCIALAGSNVFGLAREYGYYANYVKTFRRVQGASEHTHGRLCEAVGLSGGVLSQTLARIMGPAVPTEHLASLRRALVGEFETAERRFSSGQPSLLRETALIWIDVYGQTHWDITPTTPATPLSALLPVGQPSHAPSVHLAAATQIRFPALEGIHPNVLADPGFVPYVLALVVGDALRATCSAAYLPRPVEFALRVLAWARDFGLGYLPTVEGHRTKLGALITLLEPAARGGLGPTMQMADNIEQLLRELYVISRGAVEQLRPLVQLQPPPPPEVGTSLLLISMYALAARGVLQDLAERADPLIRQLEDAIVLLRLHMRTLSAFFECRFESDGRRLYAVVGDTPDRLGPWPPEAMGDAVSQYCSMYHDAKRALVASLASLRSVITETTAHLGVCDELAAQVSHEDNVLAVVRREIHGFLSVVSGIHARASKLLSGDQVPGFCFMGQFLARWRRLSACYQAARAAAGPEPVAEFVQELHDTWKGLQTERAVVVAPLVSSADQRAAAIREVMAHAPEDAPPQSPAADRVVLTSRRDLGAWGDYSLGPLGQTTAVPDSVDLSRQGLAVTLSMDWLLMNELLRVTDGVFRASAFRPLAGPESPRDLEVRDAGNSLPAPMPMDAQKPEAYGHGPRQADREGAPHSNTPVEDDEMIPEDTVAPPTDLPLTSYQ

SEQ ID NO: 79 - DNA, 1401 bp, Herpes Virus
ttgttcgggatgatgaagtttgcccacacacaccatctggtcaagcgccggggccttggggccccggccgggtacttcacccccattgccgtggacctgtggaacgtcatgtacacgttggtggtcaaatatcagcgccgataccccagttacgaccgcgaggccattacgctacactgcctctgtcgcttattaaaggtgtttacccaaaagtcccttttccccatcttcgttaccgatcgcggggtcaattgtatggagccggttgtgtttggagccaaggccatcctggcccgcacgacggcccagtgccggacggacgaggaggccagtgacgtggacgcctctccaccgccttcccccatcaccgactccagacccagctctgccttttccaacatgcgccggcgcggcacctctctggcctcggggacccgggggacggccgggtccggagccgcgctgccgtccgccgcgccctcgaagccggccctgcgtctggcgcatctgttctgtattcgcgttctccgggccctggggtacgcctacattaactcgggtcagctggaggcggacgatgcctgcgccaacctctatcacaccaacacggtcgcgtacgtgtacaccacggacactgacctcctgttgatgggctgtgatattgtgttggatattagcgcctgctacattcccacgatcaactgtcgcgatatactaaagtactttaagatgagctacccccagttcctggccctctttgtccgctgccacaccgacctccatcccaataacacctacgcctccgtggaggatgtgctgcgcgaatgtcactggacccccccgagtcgctctcagacccggcgggccatccgccgggaacacaccagctcgcgctccacggaaaccaggccccctctgccgccggccgccggcggcaccgagacgcgcgtctcgtggaccgaaattctaacccaacagatcgccggcggatacgaagacgacgaggacctccccctggatccccgggacgttaccgggggccaccccggccccaggtcgtcctcctcggagatactcaccccgcccgagctcgtccaggtcccgaacgcgcagctgctggaagagcaccgcagttatgtggccaacccgcgacgccacgtcatccacgacgccccagagtccctggactggctccccgatcccatgaccatcaccgagctggtggaacaccgctacattaagtacgtcatatcgcttatcggccccaaggagcgggggccgtggactcttctgaaacgcctgcctatctaccaggacatccgcgacgaaaacctggcgcgatctatcgtgacccggcatatcacggcccctgatatcgccgacaggtttctggagcagttg

SEQ ID NO: 80 - protein, 467 aa, Herpes Virus
LFGMMKFAHTHHLVKRRGLGAPAGYFTPIAVDLWNVMYTLVVKYQRRYPSYDREAITLHCLCRLLKVFTQKSLFPIFVTDRGVNCMEPVVFGAKAILARTTAQCRTDEEASDVDASPPPSPITDSRPSSAFSNMRRRGTSLASGTRGTAGSGAALPSAAPSKPALRLAHLFCIRVLRALGYAYINSGQLEADDACANLYHTNTVAYVYTTDTDLLLMGCDIVLDISACYIPTINCRDILKYFKMSYPQFLALFVRCHTDLHPNNTYASVEDVLRECHWTPPSRSQTRRAIRREHTSSRSTETRPPLPPAAGGTETRVSWTEILTQQIAGGYEDDEDLPLDPRDVTGGHPGPRSSSSEILTPPELVQVPNAQLLEEHRSYVANPRRHVIHDAPESLDWLPDPMTITELVEHRYIKYVISLIGPKERGPWTLLKRLPIYQDIRDENLARSIVTRHITAPDIADRFLEQL

SEQ ID NO: 81 - DNA, 1470 bp, Herpes Virus
atgggtttgttcgggatgatgaagtttgcccacacacaccatctggtcaagcgccggggccttggggccccggccgggtacttcacccccattgccgtggacctgtggaacgtcatgtacacgttggtggtcaaatatcagcgccgataccccagttacgaccgcgaggccattacgctacactgcctctgtcgcttattaaaggtgtttacccaaaagtcccttttccccatcttcgttaccgatcgcggggtcaattgtatggagccggttgtgtttggagccaaggccatcctggcccgcacgacggcccagtgccggacggacgaggaggccagtgacgtggacgcctctccaccgccttcccccatcaccgactccagacccagctctgccttttccaacatgcgccggcgcggcacctctctggcctcggggacccgggggacggccgggtccggagccgcgctgccgtccgccgcgccctcgaagccggccctgcgtctggcgcatctgttctgtattcgcgttctccgggccctggggtacgcctacattaactcgggtcagctggaggcggacgatgcctgcgccaacctctatcacaccaacacggtcgcgtacgtgtacaccacggacactgacctcctgttgatgggctgtgatattgtgttggatattagcgcctgctacattcccacgatcaactgtcgcgatatactaaagtactttaagatgagctacccccagttcctggccctctttgtccgctgccacaccgacctccatcccaataacacctacgcctccgtggaggatgtgctgcgcgaatgtcactggacccccccgagtcgctctcagacccggcgggccatccgccgggaacacaccagctcgcgctccacggaaaccaggccccctctgccgccggccgccggcggcaccgagacgcgcgtctcgtggaccgaaattctaacccaacagatcgccggcggatacgaagacgacgaggacctccccctggatccccgggacgttaccgggggccaccccggccccaggtcgtcctcctcggagatactcaccccgcccgagctcgtccaggtcccgaacgcgcagctgctggaagagcaccgcagttatgtggccaacccgcgacgccacgtcatccacgacgccccagagtccctggactggctccccgatcccatgaccatcaccgagctggtggaacaccgctacattaagtacgtcatatcgcttatcggccccaaggagcgggggccgtggactcttctgaaacgcctgcctatctaccaggacatccgcgacgaaaacctggcgcgatctatcgtgacccggcatatcacggcccctgatatcgccgacaggtttctggagcagttgcggacccaggcccccccacccgcgttctacaaggacgtcctggccaaattctgggacgagtag

SEQ ID NO: 82 - protein, 489 aa, Herpes Virus
MGLFGMMKFAHTHHLVKRRGLGAPAGYFTPIAVDLWNVMYTLVVKYQRRYPSYDREAITLHCLCRLLKVFTQKSLFPIFVTDRGVNCMEPVVFGAKAILARTTAQCRTDEEASDVDASPPPSPITDSRPSSAFSNMRRRGTSLASGTRGTAGSGAALPSAAPSKPALRLAHLFCIRVLRALGYAYINSGQLEADDACANLYHTNTVAYVYTTDTDLLLMGCDIVLDISACYIPTINCRDILKYFKMSYPQFLALFVRCHTDLHPNNTYASVEDVLRECHWTPPSRSQTRRAIRREHTSSRSTETRPPLPPAAGGTETRVSWTEILTQQIAGGYEDDEDLPLDPRDVTGGHPGPRSSSSEILTPPELVQVPNAQLLEEHRSYVANPRRHVIHDAPESLDWLPDPMTITELVEHRYIKYVISLIGPKERGPWTLLKRLPIYQDIRDENLARSIVTRHITAPDIADRFLEQLRTQAPPPAFYKDVLAKFWDE

SEQ ID NO: 83 - DNA, 1257 bp, Herpes Virus
gtgacggaaccaccccgcgtgccatcacggccaaggcgcgggatgctccgcaacgacagccaccgggccgtgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcactgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttttccagggcttgccccgaccaccgccgcgtctgtgtcatcaccgcaggcgagtctctttcccgccgccccccggaggacccagagcgacccggggttcccgggcccccgtcccccccgaccccccaacgatcccacgggccgccggccgatgaggtcgcaccggccagggtcgcgcggcccgaaaacgtctgggtgcccgtggtcacctttctgggggcgggcgcgcttgccgtcaagacggtgcgagaacatgcccggggaacgccgggcccgggcctgccgctgtggccccaggtgtttctcggaggccatgtggcggtggccctgacggagctgtgtcaggcgcttccgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctg

SEQ ID NO: 84 - protein, 419 aa, Herpes Virus
VTEPPRVPSRPRRGMLRNDSHRAVSPEDGQGRVDDGRPHLACVGALARGFMHIWLQAATLGFAGSVVMSRGPYANAASGAFAVGCAVLGFMRAPPPLARPTARIYAWLKLAAGGAALVLWSLGEPGTQPGAPAPGPATQCLALGAAYAALLVLADDVYPLFLLAPGPLFVGTLGMVVGGLTIGGSARYWWIGGPAAAALAAAVLAGPGATTARDCFSRACPDHRRVCVITAGESLSRRPPEDPERPGVPGPPSPPTPQRSHGPPADEVAPARVARPENVWVPVVTFLGAGALAVKTVREHARGTPGPGLPLWPQVFLGGHVAVALTELCQALPPWDLTDPLLFVHAGLQVINLGLVFRFSEVVVYAALGGAVWISLAQVLGLRRRLHRKDPGDGARLAATLRGLFFSVYALGFGVGVLL

SEQ ID NO: 85 - DNA, 1305 bp, Herpes Virus
atgtggggcgtgacggaaccaccccgcgtgccatcacggccaaggcgcgggatgctccgcaacgacagccaccgggccgtgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcactgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttttccagggcttgccccgaccaccgccgcgtctgtgtcatcaccgcaggcgagtctctttcccgccgccccccggaggacccagagcgacccggggttcccgggcccccgtcccccccgaccccccaacgatcccacgggccgccggccgatgaggtcgcaccggccagggtcgcgcggcccgaaaacgtctgggtgcccgtggtcacctttctgggggcgggcgcgcttgccgtcaagacggtgcgagaacatgcccggggaacgccgggcccgggcctgccgctgtggccccaggtgtttctcggaggccatgtggcggtggccctgacggagctgtgtcaggcgcttccgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattga

SEQ ID NO: 86 - protein, 434 aa, Herpes Virus
MWGVTEPPRVPSRPRRGMLRNDSHRAVSPEDGQGRVDDGRPHLACVGALARGFMHIWLQAATLGFAGSVVMSRGPYANAASGAFAVGCAVLGFMRAPPPLARPTARIYAWLKLAAGGAALVLWSLGEPGTQPGAPAPGPATQCLALGAAYAALLVLADDVYPLFLLAPGPLFVGTLGMVVGGLTIGGSARYWWIGGPAAAALAAAVLAGPGATTARDCFSRACPDHRRVCVITAGESLSRRPPEDPERPGVPGPPSPPTPQRSHGPPADEVAPARVARPENVWVPVVTFLGAGALAVKTVREHARGTPGPGLPLWPQVFLGGHVAVALTELCQALPPWDLTDPLLFVHAGLQVINLGLVFRFSEVVVYAALGGAVWISLAQVLGLRRRLHRKDPGDGARLAATLRGLFFSVYALGFGVGVLLCPPGSTGGRSGD

SEQ ID NO: 87 - DNA, 711 bp, Herpes Virus
gtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctgg

SEQ ID NO: 88 - protein, 237 aa, Herpes Virus
VVLWSLLWLGAGVSGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAW

SEQ ID NO: 89 - DNA, 1536 bp, Herpes Virus
atggccccggggcgggtgggccttgccgtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccaccaggtgtttaacccgggccagatcgacacgcagacgcacgagcaccccgacgggttcaccacagtctctaccgtgacctccgaggctgtcggcggccaggtccccccgcggaccttcacctgccagatgacgtggcatcgcgactccgtgacgttctcgcgacgcaatgccaccgggctggccctggtgctgccgcggccaaccatcaccatggaatttggggtccgcattgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagaacaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaa

SEQ ID NO: 90 - protein, 511 aa, Herpes Virus
MAPGRVGLAVVLWSLLWLGAGVSGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFVWFEDDHQVFNPGQIDTQTHEHPDGFTTVSTVTSEAVGGQVPPRTFTCQMTWHRDSVTFSRRNATGLALVLPRPTITMEFGVRIVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 91 - DNA, 843 bp, Herpes Virus
gatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccaga

SEQ ID NO: 92 - protein, 281 aa, Herpes Virus
DEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPR

SEQ ID NO: 93 - DNA, 906 bp, Herpes Virus
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagtga

SEQ ID NO: 94 - protein, 301 aa, Herpes Virus
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 95 - DNA, 1023 bp, Herpes Virus
gactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgac

SEQ ID NO: 96 - protein, 341 aa, Herpes Virus
DWFPCYDDAGDEEWAEDPGAMDTSHDPPDDEVAYFDLCHEVGPTAEPRETDSPVCSCTDKIGLRVCMPVPAPYVVHGSLTMRGVARVIQQAVLLDRDFVEAIGSYVKNFLLIDTGVYAHGHSLRLPYFAKIAPDGPACGRLLPVFVIPPACKDVPAFVAAHADPRRFHFHAPPTYLASPREIRVLHSLGGDYVSFFERKASRNALEHFGRRETLTEVLGRYNVQPDAGGTVEGFASELLGRIVACIETHFPEHAGEYQAVSVRRAVSKDDWVLLQLVPVRGTLQQSLSCLRFKHGRASRATARTFVALSVGANNRLCVSLCQQCFAAKCDSNRLHTLFTID

SEQ ID NO: 97 - DNA, 3177 bp, Herpes Virus
atggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggtgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggccccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttga

SEQ ID NO: 98 - protein, 1058 aa, Herpes Virus
MGQEDGNRGERRAAGTPVEVTALYATDGCVITSSIALLTNSLLGAEPVYIFSYDAYTHDGRADGPTEQDRFEESRALYQASGGLNGDSFRVTFCLLGTEVGGTHQARGRTRPMFVCRFERADDVAALQDALAHGTPLQPDHIAATLDAEATFALHANMILALTVAINNASPRTGRDAAAAQYDQGASLRSLVGRTSLGQRGLTTLYVHHEVRVLAAYRRAYYGSAQSPFWFLSKFGPDEKSLVLTTRYYLLQAQRLGGAGATYDLQAIKDICATYAIPHAPRPDTVSAASLTSFAAITRFCCTSQYARGAAAAGFPLYVERRIAADVRETSALEKFITHDRSCLRVSDREFITYIYLAHFECFSPPRLATHLRAVTTHDPNPAASTEQPSPLGREAVEQFFCHVRAQLNIGEYVKHNVTPRETVLDGDTAKAYLRARTYAPGALTPAPAYCGAVDSATKMMGRLADAEKLLVPRGWPAFAPASPGEDTAGGTPPPQTCGIVKRLLRLAATEQQGPTPPAIAALIRNAAVQTPLPVYRISMVPTGQAFAALAWDDWARITRDARLAEAVVSAEAAAHPDHGALGRRLTDRIRAQGPVMPPGGLDAGGQMYVNRNEIFNGALAITNIILDLDIALKEPVPFRRLHEALGHFRRGALAAVQLLFPAARVDPDAYPCYFFKSACRPGPASVGSGSGLGNDDDGDWFPCYDDAGDEEWAEDPGAMDTSHDPPDDEVAYFDLCHEVGPTAEPRETDSPVCSCTDKIGLRVCMPVPAPYVVHGSLTMRGVARVIQQAVLLDRDFVEAIGSYVKNFLLIDTGVYAHGHSLRLPYFAKIAPDGPACGRLLPVFVIPPACKDVPAFVAAHADPRRFHFHAPPTYLASPREIRVLHSLGGDYVSFFERKASRNALEHFGRRETLTEVLGRYNVQPDAGGTVEGFASELLGRIVACIETHFPEHAGEYQAVSVRRAVSKDDWVLLQLVPVRGTLQQSLSCLRFKHGRASRATARTFVALSVGANNRLCVSLCQQCFAAKCDSNRLHTLFTIDAGTPCSPSVPCSTSQPSS

SEQ ID NO: 99 - DNA, 701 bp, Herpes Virus
ttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacgaccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttc

SEQ ID NO: 100 - protein, 233 aa, Herpes Virus
LVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQEC

SEQ ID NO: 101 - DNA, 1539 bp, Herpes Virus
atggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacgaccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 102 - protein, 512 aa, Herpes Virus
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 103 - DNA, 974 bp, Herpes Virus
cggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtgtcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccatccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaaataccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctggggga

SEQ ID NO: 104 - protein, 324 aa, Herpes Virus
RRMACRKFCRVYGGQGRRKEEAVPPETKPSRVFPHGPFYTPAEDACLDSPPPETPKPSHTTPPSEAERLCHLQEILAQMYGNQDYPIEDDPSADAADDVDEDAPDDVAYPEEYAEELFLPGDATGPLIGANDHIPPPCGASPPGIRRRSRDEIGATGFTAEELDAMDREAARAISRGGKPPSTMAKLVTGMGFTIHGALTPGSEGCVFDSSHPDYPQRVIVKAGWYTSTSHEARLLRRLDHPAILPLLDLHVVSGVTCLVLPKYQADLYTYLSRRLNPLGRPQIAAVSRQLLSAVDYIHRQGIIHRDIKTENIFINTPEDICLG

SEQ ID NO: 105 - DNA, 1446 bp, Herpes Virus
atggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtgtcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccatccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctgggggactttggcgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccgtacacccgaccggcctggacccgctactacaagatggacatagacgtcgaatatctggtttgcaaagccctcaccttcgacggcgcgcttcgccccagcgccgcagagctgctttgtttgccgctgtttcaacagaaatga

SEQ ID NO: 106 - protein, 481 aa, Herpes Virus
MACRKFCRVYGGQGRRKEEAVPPETKPSRVFPHGPFYTPAEDACLDSPPPETPKPSHTTPPSEAERLCHLQEILAQMYGNQDYPIEDDPSADAADDVDEDAPDDVAYPEEYAEELFLPGDATGPLIGANDHIPPPCGASPPGIRRRSRDEIGATGFTAEELDAMDREAARAISRGGKPPSTMAKLVTGMGFTIHGALTPGSEGCVFDSSHPDYPQRVIVKAGWYTSTSHEARLLRRLDHPAILPLLDLHVVSGVTCLVLPKYQADLYTYLSRRLNPLGRPQIAAVSRQLLSAVDYIHRQGIIHRDIKTENIFINTPEDICLGDFGAACFVQGSRSSPFPYGIAGTIDTNAPEVLAGDPYTTTVDIWSAGLVIFETAVHNASLFSAPRGPKRGPCDSQITRIIRQAQVHVDEFSPHPESRLTSRYRSRAAGNNRPPYTRPAWTRYYKMDIDVEYLVCKALTFDGALRPSAAELLCLPLFQQK

SEQ ID NO: 107 - DNA, 261 bp, Herpes Virus
gtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcatgtctgcttgagctcctgcgtcgtacgtgccgcgggtgggggcgttaccatccctacatggacccagttgtcgta

SEQ ID NO: 108 - protein, 87 aa, Herpes Virus
VWHLGLLGSLVGAVLAATHRGPAANTTDPLTHAPVSPHPSPLGGFAVPLVVGGLCAVVLGAACLLELLRRTCRGWGRYHPYMDPVVV

SEQ ID NO: 109 - DNA, 279 bp, Herpes Virus
atgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcatgtctgcttgagctcctgcgtcgtacgtgccgcgggtgggggcgttaccatccctacatggacccagttgtcgtataa

SEQ ID NO: 110 - protein, 92 aa, Herpes Virus
MSLRAVWHLGLLGSLVGAVLAATHRGPAANTTDPLTHAPVSPHPSPLGGFAVPLVVGGLCAVVLGAACLLELLRRTCRGWGRYHPYMDPVVV

SEQ ID NO: 111 - DNA, 1381 bp, Herpes Virus
caccgacgaatcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactagatacccccccttaatgggtgc

SEQ ID NO: 112 - protein, 394 aa, Herpes Virus
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 113 - DNA, 1092 bp, Herpes Virus
attttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcata

SEQ ID NO: 114 - protein, 364 aa, Herpes Virus
ILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRI

SEQ ID NO: 115 - DNA, 1185 bp, Herpes Virus
atggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactag

SEQ ID NO: 116 - protein, 394 aa, Herpes Virus; identical to SEQ ID NO: 112